(19) United States
(12) Patent Application Publication
(10) Pub. No.: US 2025/0373238 A1
(43) Pub. Date: Dec. 4, 2025

(54) TECHNIQUES FOR FILTERING AGGRESSOR SIGNALS FROM BIOPOTENTIAL SIGNALS, AND CIRCUITS IMPLEMENTING THE TECHNIQUES

(71) Applicant: Meta Platforms Technologies, LLC, Menlo Park, CA (US)

(72) Inventors: Filipp Demenschonok, Bothell, WA (US); Sayyed Mahdi Kashmiri, San Jose, CA (US); Baolin Yi, Cupertino, CA (US); Emrah Adamey, Vancouver (CA); Eric VanWyk, Seattle, WA (US)

(21) Appl. No.: 19/221,509

(22) Filed: May 29, 2025

Related U.S. Application Data
(60) Provisional application No. 63/653,942, filed on May 30, 2024.

(51) Int. Cl. H03K 5/1252; H03F 3/45; H03M 1/00

(52) U.S. Cl. CPC H03K 5/1252; H03F 3/45475; H03M 1/001; H03F 2200/261

(57) ABSTRACT

The various implementations described herein include techniques and apparatuses for multi-channel biopotential signal acquisition, biopotential signal pre-processing, and adaptive signal conditioning. In one aspect, an analog frontend circuit includes an instrumentation amplifier (INA) and an analog-to-digital circuit (ADC) in a forward signal path. A digital circuit receives input from the ADC. An adaptive baseline tracking and compensation circuit tracks and compensates moving motion artifact driven changes. A power line interference (PLI) detection and compensation circuit tracks a desired number of PLI harmonics, and magnitude, phase and frequency for the PLI harmonics in real time. A digital-to-analog converter (DAC) circuit combines output of the adaptive baseline tracking and compensation circuit and the PLI detection and compensation circuit to output an analog output. A passive filter receives the analog output and drives an analog compensation signal at an input of the INA.

### RELATED APPLICATIONS

This application claims priority to U.S. Provisional Patent Application No. 63/653,942, filed May 30, 2024, titled “Techniques For Filtering Aggressor Signals From Biopotential Signals, And Circuits Implementing The Techniques,” which is hereby incorporated by reference in its entirety.

### TECHNICAL FIELD

The present disclosure relates generally to biopotential signal (e.g., muscular response or electromyography) acquisition and interpretation, including, but not limited to, techniques and apparatuses for multi-channel biopotential signal acquisition, biopotential signal pre-processing, and/or adaptive signal conditioning.

### BACKGROUND

The present disclosure relates generally to biopotential signal (e.g., muscular response or electromyography) acquisition and interpretation, including, but not limited to, techniques and apparatuses for multi-channel biopotential signal acquisition, biopotential signal pre-processing, and/or adaptive signal conditioning.

Biopotential signals (e.g., EMG signals) can be a useful means of detecting user movements and gestures (e.g., in-air hand gestures during which a user might pinch together a finger and thumb). The detection and interpretation of user movements and gestures can enable a system (such as an artificial-reality system) to be responsive to the user movements and gestures. However, conventional means of detecting (sensing) biopotential signals are susceptible to noise, such as motion artifacts, baseline wandering, and power-line induced noise. These noise sources can lead to erroneous results and poor-quality human-machine interactions.

Power consumption for systems that process biopotential signals can also be an issue, e.g., because in some instances the machine-learning models used to process and categorize the biopotential signals can require a relatively high amount of power to function. Thus, low-power techniques used to wake-up these machine-learning models at appropriate times are needed. An analog-based (and low-power consumption) technique used to wake-up digital signal-processing components would be desirable to address this issue. As such, there is a need to address one or more of the above-identified challenges. A brief summary of solutions to the issues noted above are described below.

### SUMMARY

The apparatuses, systems, devices (e.g., wearable devices) and methods described herein address at least some of the above-mentioned drawbacks by reducing and/or compensating for noise. In accordance with some embodiments, an apparatus is provided for adaptive signal conditioning for biopotential acquisition. The apparatus includes an analog circuit configured to amplify biopotential signals, and a mixed-signal circuit (e.g., which can include an adaptive digital algorithm) coupled to the analog circuit and configured to suppress aggressor signals comprising baseline wandering signals and power-line-induced noise in the biopotential signals before amplification of the biopotential signals.

In accordance with some embodiments, an apparatus is provided for processing biopotential signals. The apparatus includes an analog frontend circuit comprising an instrumentation amplifier (INA) and an analog-to-digital circuit (ADC) in a forward signal path. The apparatus also includes a digital circuit coupled to the analog frontend circuit and configured to receive input from the ADC. The digital circuit includes an adaptive baseline tracking and compensation circuit configured to track and compensate motion artifact driven changes. The digital circuit also includes a power line interference (PLI) detection and compensation circuit configured to (i) track a desired number of PLI harmonics, and (ii) track magnitude, phase, and frequency for the PLI harmonics in real time. The digital circuit includes an a digital-to-analog converter (DAC) circuit configured to combine output of the adaptive baseline tracking and compensation circuit and the PLI detection and compensation circuit to output an analog output. The digital circuit includes a passive filter configured to receive the analog output and drive an analog compensation signal at an input of the INA to counteract present artifacts at the input.

In some embodiments, a computing device (e.g., a wrist-wearable device or a head-mounted device, or an intermediary device, such as a smartphone or desktop or laptop computer that can be configured to coordinate operations at one or more wearable devices) includes one or more of the apparatuses, circuits, and/or systems described herein.

Thus, methods, apparatuses, devices, and systems are disclosed for biopotential signal (e.g., neuromuscular signal, such as electromyography signal) detection and interpretation. Such methods, apparatuses, devices, and systems may complement or replace conventional methods for neuromuscular-signal detection and interpretation.

The features and advantages described in the specification are not necessarily all inclusive and, in particular, certain additional features and advantages will be apparent to one of ordinary skill in the art in view of the drawings, specification, and claims. Moreover, it should be noted that the language used in the specification has been principally selected for readability and instructional purposes.

Having summarized the above example aspects, a brief description of the drawings will now be presented.

### BRIEF DESCRIPTION OF THE DRAWINGS

For a better understanding of the various described embodiments, reference should be made to the Detailed Description below, in conjunction with the following drawings in which like reference numerals refer to corresponding parts throughout the figures.

FIG. 1 is a schematic diagram of an example adaptive analog front-end architecture, according to some embodiments.

FIG. 2A is a schematic diagram of an example current-feedback instrumentation amplifier, according to some embodiments.

FIG. 2B is a detailed block diagram of the example current-feedback instrumentation amplifier, according to some embodiments.

FIG. 2C is a block diagram of the example current-feedback instrumentation amplifier with connections to a bipolar electrode pair for biopotential sensing, according to some embodiments.

FIG. 2D is a schematic diagram of the example current-feedback instrumentation amplifier including an analog servo loop, according to some embodiments.

FIG. 2E is a schematic diagram of the example current-feedback instrumentation amplifier including a digital loop, according to some embodiments.

FIG. 3 is a schematic diagram of an example circuit with a Digital-to-analog converter (DAC), according to some embodiments.

FIG. 4A is a block diagram of an example system using a hybrid DAC, according to some embodiments.

FIG. 4B is a detailed view of the hybrid DAC, according to some embodiments.

FIG. 4C shows example graph plots corresponding to raw DAC code coarse and raw DAC code fine, respectively, according to some embodiments.

FIG. 4D shows graph plots corresponding to corrected DAC code coarse and corrected DAC code fine, respectively, according to some embodiments.

FIG. 4E shows example graph plots corresponding to final noise shaped DAC code and pre low pass filtered analog hybrid DAC output, respectively, according to some embodiments.

FIG. 5A shows a graph plot comparing digital output signal spectra (magnitude in dB versus frequency in Hz) for traditional analog frontend versus adaptive analog frontend, according to some embodiments.

FIG. 5B shows a graph plot comparing pre-low pass filter (LPF) analog DAC signal and post-LPF analog DAC signal, according to some embodiments.

FIG. 6A is a block diagram of an example system that includes an motion artifact algorithm, according to some embodiments.

FIG. 6B is a block diagram showing a detailed view of the example system shown in FIG. 6A, according to some embodiments.

FIG. 6C shows graph plots for example operation of the motion artifact algorithm, according to some embodiments.

FIG. 6D shows graph plots for example operation of the motion artifact algorithm, according to some embodiments.

FIG. 6E shows graph plots for example operation of output stitching, according to some embodiments.

FIG. 7A shows block diagrams of example internal components for power line interference (PLI) estimation algorithm, according to some embodiments.

FIG. 7B is a block diagram of an example system for implementing PLI tracking and compensation algorithm, according to some embodiments.

FIGS. 7C, 7D, and 7E show graph plots for example operations of the PLI algorithm, according to some embodiments.

FIG. 7F is a block diagram of another example system for implementing the PLI algorithm, according to some embodiments.

FIG. 7G shows graph plots for example simulation of the algorithm described above in reference to FIG. 7F when recovering from a large motion artifact, according to some embodiments.

FIG. 7H shows graph plots for example operation for disabling the rate of change monitor signal, according to some embodiments.

FIG. 7I is a block diagram of another example system 750 for implementing the PLI algorithm 124, according to some embodiments.

FIG. 8A is a block diagram of an example system for EMG-based gesture recognition, according to some embodiments.

FIG. 8B is a block diagram of an example digital equivalent of an analog 1-D correlator coupled to an analog front-end, according to some embodiments.

FIG. 9 shows an example envelope template in accordance with some embodiments.

FIG. 10A shows a graph plot for example allowed mismatch before error versus prediction, according to some embodiments.

FIG. 10B is a schematic diagram of example plots of intervals update in a traditional successive approximation register (SAR) ADC, an improved SAR with good prediction and an improved SAR with bad prediction, according to some embodiments.

FIGS. 11A, 11B, 11C-1, and 11C-2 illustrate example MR and AR systems, in accordance with some embodiments.

In accordance with common practice, the various features illustrated in the drawings may not be drawn to scale. Accordingly, the dimensions of the various features may be arbitrarily expanded or reduced for clarity. In addition, some of the drawings may not depict all of the components of a given system, method, or device. Finally, like reference numerals may be used to denote like features throughout the specification and figures.

### DETAILED DESCRIPTION

Numerous details are described herein to provide a thorough understanding of the example embodiments illustrated in the accompanying drawings. However, some embodiments may be practiced without many of the specific details, and the scope of the claims is only limited by those features and aspects specifically recited in the claims. Furthermore, well-known processes, components, and materials have not necessarily been described in exhaustive detail so as to avoid obscuring pertinent aspects of the embodiments described herein.

### Overview

Embodiments of this disclosure can include or be implemented in conjunction with various types of extended-realities (XRs) such as mixed-reality (MR) and augmented-reality (AR) systems. MRs and ARs, as described herein, are any superimposed functionality and/or sensory-detectable presentation provided by MR and AR systems within a user's physical surroundings. Such MRs can include and/or represent virtual realities (VRs) and VRs in which at least some aspects of the surrounding environment are reconstructed within the virtual environment (e.g., displaying virtual reconstructions of physical objects in a physical environment to avoid the user colliding with the physical objects in a surrounding physical environment). In the case of MRs, the surrounding environment that is presented through a display is captured via one or more sensors configured to capture the surrounding environment (e.g., a camera sensor, time-of-flight (ToF) sensor). While a wearer of an MR headset can see the surrounding environment in full detail, they are seeing a reconstruction of the environment reproduced using data from the one or more sensors (i.e., the physical objects are not directly viewed by the user). An MR headset can also forgo displaying reconstructions of objects in the physical environment, thereby providing a user with an entirely VR experience. An AR system, on the other hand, provides an experience in which information is provided, e.g., through the use of a waveguide, in conjunction with the direct viewing of at least some of the surrounding environment through a transparent or semi-transparent waveguide(s) and/or lens(es) of the AR glasses. Throughout this application, the term “extended reality (XR)” is used as a catchall term to cover both ARs and MRs. In addition, this application also uses, at times, a head-wearable device or headset device as a catchall term that covers XR headsets such as AR glasses and MR headsets.

As alluded to above, an MR environment, as described herein, can include, but is not limited to, non-immersive, semi-immersive, and fully immersive VR environments. As also alluded to above, AR environments can include marker-based AR environments, markerless AR environments, location-based AR environments, and projection-based AR environments. The above descriptions are not exhaustive and any other environment that allows for intentional environmental lighting to pass through to the user would fall within the scope of an AR, and any other environment that does not allow for intentional environmental lighting to pass through to the user would fall within the scope of an MR.

The AR and MR content can include video, audio, haptic events, sensory events, or some combination thereof, any of which can be presented in a single channel or in multiple channels (such as stereo video that produces a three-dimensional effect to a viewer). Additionally, AR and MR can also be associated with applications, products, accessories, services, or some combination thereof, which are used, for example, to create content in an AR or MR environment and/or are otherwise used in (e.g., to perform activities in) AR and MR environments.

Interacting with these AR and MR environments described herein can occur using multiple different modalities and the resulting outputs can also occur across multiple different modalities. In one example AR or MR system, a user can perform a swiping in-air hand gesture to cause a song to be skipped by a song-providing application programming interface (API) providing playback at, for example, a home speaker.

A hand gesture, as described herein, can include an in-air gesture, a surface-contact gesture, and or other gestures that can be detected and determined based on movements of a single hand (e.g., a one-handed gesture performed with a user's hand that is detected by one or more sensors of a wearable device (e.g., electromyography (EMG) and/or inertial measurement units (IMUs) of a wrist-wearable device, and/or one or more sensors included in a smart textile wearable device) and/or detected via image data captured by an imaging device of a wearable device (e.g., a camera of a head-wearable device, an external tracking camera setup in the surrounding environment)). “In-air” generally includes gestures in which the user's hand does not contact a surface, object, or portion of an electronic device (e.g., a head-wearable device or other communicatively coupled device, such as the wrist-wearable device), in other words the gesture is performed in open air in 3D space and without contacting a surface, an object, or an electronic device. Surface-contact gestures (contacts at a surface, object, body part of the user, or electronic device) more generally are also contemplated in which a contact (or an intention to contact) is detected at a surface (e.g., a single- or double-finger tap on a table, on a user's hand or another finger, on the user's leg, a couch, a steering wheel). The different hand gestures disclosed herein can be detected using image data and/or sensor data (e.g., neuromuscular signals sensed by one or more biopotential sensors (e.g., EMG sensors) or other types of data from other sensors, such as proximity sensors, ToF sensors, sensors of an IMU, capacitive sensors, strain sensors) detected by a wearable device worn by the user and/or other electronic devices in the user's possession (e.g., smartphones, laptops, imaging devices, intermediary devices, and/or other devices described herein).

The input modalities as alluded to above can be varied and are dependent on a user's experience. For example, in an interaction in which a wrist-wearable device is used, a user can provide inputs using in-air or surface-contact gestures that are detected using neuromuscular signal sensors of the wrist-wearable device. In the event that a wrist-wearable device is not used, alternative and entirely interchangeable input modalities can be used instead, such as camera(s) located on the headset/glasses or elsewhere to detect in-air or surface-contact gestures or inputs at an intermediary processing device (e.g., through physical input components (e.g., buttons and trackpads)). These different input modalities can be interchanged based on both desired user experiences, portability, and/or a feature set of the product (e.g., a low-cost product may not include hand-tracking cameras).

While the inputs are varied, the resulting outputs stemming from the inputs are also varied. For example, an in-air gesture input detected by a camera of a head-wearable device can cause an output to occur at a head-wearable device or control another electronic device different from the head-wearable device. In another example, an input detected using data from a neuromuscular signal sensor can also cause an output to occur at a head-wearable device or control another electronic device different from the head-wearable device. While only a couple examples are described above, one skilled in the art would understand that different input modalities are interchangeable along with different output modalities in response to the inputs.

Specific operations described above may occur as a result of specific hardware. The devices described are not limiting and features on these devices can be removed or additional features can be added to these devices. The different devices can include one or more analogous hardware components. For brevity, analogous devices and components are described herein. Any differences in the devices and components are described below in their respective sections.

As described herein, a processor (e.g., a central processing unit (CPU) or microcontroller unit (MCU)), is an electronic component that is responsible for executing instructions and controlling the operation of an electronic device (e.g., a wrist-wearable device, a head-wearable device, a handheld intermediary processing device (HIPD), a smart textile-based garment, or other computer system). There are various types of processors that may be used interchangeably or specifically required by embodiments described herein. For example, a processor may be (i) a general processor designed to perform a wide range of tasks, such as running software applications, managing operating systems, and performing arithmetic and logical operations; (ii) a microcontroller designed for specific tasks such as controlling electronic devices, sensors, and motors; (iii) a graphics processing unit (GPU) designed to accelerate the creation and rendering of images, videos, and animations (e.g., VR animations, such as three-dimensional modeling); (iv) a field-programmable gate array (FPGA) that can be programmed and reconfigured after manufacturing and/or customized to perform specific tasks, such as signal processing, cryptography, and machine learning; or (v) a digital signal processor (DSP) designed to perform mathematical operations on signals such as audio, video, and radio waves. One of skill in the art will understand that one or more processors of one or more electronic devices may be used in various embodiments described herein.

As described herein, controllers are electronic components that manage and coordinate the operation of other components within an electronic device (e.g., controlling inputs, processing data, and/or generating outputs). Examples of controllers can include (i) microcontrollers, including small, low-power controllers that are commonly used in embedded systems and Internet of Things (IoT) devices; (ii) programmable logic controllers (PLCs) that may be configured to be used in industrial automation systems to control and monitor manufacturing processes; (iii) system-on-a-chip (SoC) controllers that integrate multiple components such as processors, memory, I/O interfaces, and other peripherals into a single chip; and/or (iv) DSPs. As described herein, a graphics module is a component or software module that is designed to handle graphical operations and/or processes and can include a hardware module and/or a software module.

As described herein, memory refers to electronic components in a computer or electronic device that store data and instructions for the processor to access and manipulate. The devices described herein can include volatile and non-volatile memory. Examples of memory can include (i) random access memory (RAM), such as DRAM, SRAM, DDR RAM or other random access solid state memory devices, configured to store data and instructions temporarily; (ii) read-only memory (ROM) configured to store data and instructions permanently (e.g., one or more portions of system firmware and/or boot loaders); (iii) flash memory, magnetic disk storage devices, optical disk storage devices, other non-volatile solid state storage devices, which can be configured to store data in electronic devices (e.g., universal serial bus (USB) drives, memory cards, and/or solid-state drives (SSDs)); and (iv) cache memory configured to temporarily store frequently accessed data and instructions. Memory, as described herein, can include structured data (e.g., SQL databases, MongoDB databases, GraphQL data, or JSON data). Other examples of memory can include (i) profile data, including user account data, user settings, and/or other user data stored by the user; (ii) sensor data detected and/or otherwise obtained by one or more sensors; (iii) media content data including stored image data, audio data, documents, and the like; (iv) application data, which can include data collected and/or otherwise obtained and stored during use of an application; and/or (v) any other types of data described herein.

As described herein, a power system of an electronic device is configured to convert incoming electrical power into a form that can be used to operate the device. A power system can include various components, including (i) a power source, which can be an alternating current (AC) adapter or a direct current (DC) adapter power supply; (ii) a charger input that can be configured to use a wired and/or wireless connection (which may be part of a peripheral interface, such as a USB, micro-USB interface, near-field magnetic coupling, magnetic inductive and magnetic resonance charging, and/or radio frequency (RF) charging); (iii) a power-management integrated circuit, configured to distribute power to various components of the device and ensure that the device operates within safe limits (e.g., regulating voltage, controlling current flow, and/or managing heat dissipation); and/or (iv) a battery configured to store power to provide usable power to components of one or more electronic devices.

As described herein, peripheral interfaces are electronic components (e.g., of electronic devices) that allow electronic devices to communicate with other devices or peripherals and can provide a means for input and output of data and signals. Examples of peripheral interfaces can include (i) USB and/or micro-USB interfaces configured for connecting devices to an electronic device; (ii) Bluetooth interfaces configured to allow devices to communicate with each other, including Bluetooth low energy (BLE); (iii) near-field communication (NFC) interfaces configured to be short-range wireless interfaces for operations such as access control; (iv) pogo pins, which may be small, spring-loaded pins configured to provide a charging interface; (v) wireless charging interfaces; (vi) global-positioning system (GPS) interfaces; (vii) Wi-Fi interfaces for providing a connection between a device and a wireless network; and (viii) sensor interfaces.

As described herein, sensors are electronic components (e.g., in and/or otherwise in electronic communication with electronic devices, such as wearable devices) configured to detect physical and environmental changes and generate electrical signals. Examples of sensors can include (i) imaging sensors for collecting imaging data (e.g., including one or more cameras disposed on a respective electronic device, such as a simultaneous localization and mapping (SLAM) camera); (ii) biopotential-signal sensors (used interchangeably with neuromuscular-signal sensors); (iii) IMUs for detecting, for example, angular rate, force, magnetic field, and/or changes in acceleration; (iv) heart rate sensors for measuring a user's heart rate; (v) peripheral oxygen saturation (SpO2) sensors for measuring blood oxygen saturation and/or other biometric data of a user; (vi) capacitive sensors for detecting changes in potential at a portion of a user's body (e.g., a sensor-skin interface) and/or the proximity of other devices or objects; (vii) sensors for detecting some inputs (e.g., capacitive and force sensors); and (viii) light sensors (e.g., ToF sensors, infrared light sensors, or visible light sensors), and/or sensors for sensing data from the user or the user's environment. As described herein biopotential-signal-sensing components are devices used to measure electrical activity within the body (e.g., biopotential-signal sensors). Some types of biopotential-signal sensors include (i) electroencephalography (EEG) sensors configured to measure electrical activity in the brain to diagnose neurological disorders; (ii) electrocardiography (ECG or EKG) sensors configured to measure electrical activity of the heart to diagnose heart problems; (iii) EMG sensors configured to measure the electrical activity of muscles and diagnose neuromuscular disorders; (iv) electrooculography (EOG) sensors configured to measure the electrical activity of eye muscles to detect eye movement and diagnose eye disorders.

As described herein, an application stored in memory of an electronic device (e.g., software) includes instructions stored in the memory. Examples of such applications include (i) games; (ii) word processors; (iii) messaging applications; (iv) media-streaming applications; (v) financial applications; (vi) calendars; (vii) clocks; (viii) web browsers; (ix) social media applications; (x) camera applications; (xi) web-based applications; (xii) health applications; (xiii) AR and MR applications; and/or (xiv) any other applications that can be stored in memory. The applications can operate in conjunction with data and/or one or more components of a device or communicatively coupled devices to perform one or more operations and/or functions.

As described herein, communication interface modules can include hardware and/or software capable of data communications using any of a variety of custom or standard wireless protocols (e.g., IEEE 802.15.4, Wi-Fi, ZigBec, 6LoWPAN, Thread, Z-Wave, Bluetooth Smart, ISA100.11a, WirelessHART, or MiWi), custom or standard wired protocols (e.g., Ethernet or HomePlug), and/or any other suitable communication protocol, including communication protocols not yet developed as of the filing date of this document. A communication interface is a mechanism that enables different systems or devices to exchange information and data with each other, including hardware, software, or a combination of both hardware and software. For example, a communication interface can refer to a physical connector and/or port on a device that enables communication with other devices (e.g., USB, Ethernet, HDMI, or Bluetooth). A communication interface can refer to a software layer that enables different software programs to communicate with each other (e.g., APIs and protocols such as HTTP and TCP/IP).

As described herein, a graphics module is a component or software module that is designed to handle graphical operations and/or processes and can include a hardware module and/or a software module.

As described herein, non-transitory computer-readable storage media are physical devices or storage medium that can be used to store electronic data in a non-transitory form (e.g., such that the data is stored permanently until it is intentionally deleted and/or modified).

The present disclosure includes biopotential (e.g., electromyography (EMG)) acquisition and/or measurement circuits (e.g., integrated circuits such as application-specific integrated circuits (ASIC)) and apparatuses. The circuits and apparatuses include various active noise and feedback mechanisms to address various non-idealities in the biopotential (e.g., EMG) signal (e.g., power line noise, motion artifacts and offsets).

The circuits and apparatuses described herein eliminate, reduce, suppress, or mitigate noise sources (sometimes called aggressors), such as electrode offset (e.g., due to the human motion artifacts), baseline wandering, and power line (e.g., a 50/60 Hz power line) induced noise before amplification of the small biopotential signals. The noise signals such as baseline wander and interference are generally much larger than the biopotential signal of interest. For example, a noise signal may be hundreds of mV whereas the biopotential signal may be in the range of tens of microvolts to a few millivolts.

If both the noise signals and the signal of interest go through a signal conditioning analog frontend with a same (or similar) rate of amplification, the signal path saturates due to the noise signals resulting in significant degradation of functionality and performance. By suppressing the noise signals before amplification of the signal of interest, a larger gain can be utilized that enables a better SNR and system optimization in terms of power consumption and size.

In some embodiments, an analog frontend (AFE) comprises an instrumentation amplifier (INA), a programmable gain amplifier (PGA) 118 and an ADC 120 in the forward signal path. The output of the ADC feeds a set of adaptive algorithms in the digital domain, which perform functions including: (i) adaptive baseline tracking and compensation to track and compensate slow as well as large and fast moving motion artifact (MA) driven changes and (ii) power line interference (PLI) detection and compensation, tracking a desired number of PLI harmonics (e.g., up to 7) and tracking magnitude, phase and frequency for PLI harmonics in real time.

In some embodiments, the output of the above functions are combined to drive a digital-to-analog converter (DAC), whose analog output is then filtered by means of a passive filter to drive an analog compensation signal at the input of the INA to counteract present artifacts at the input.

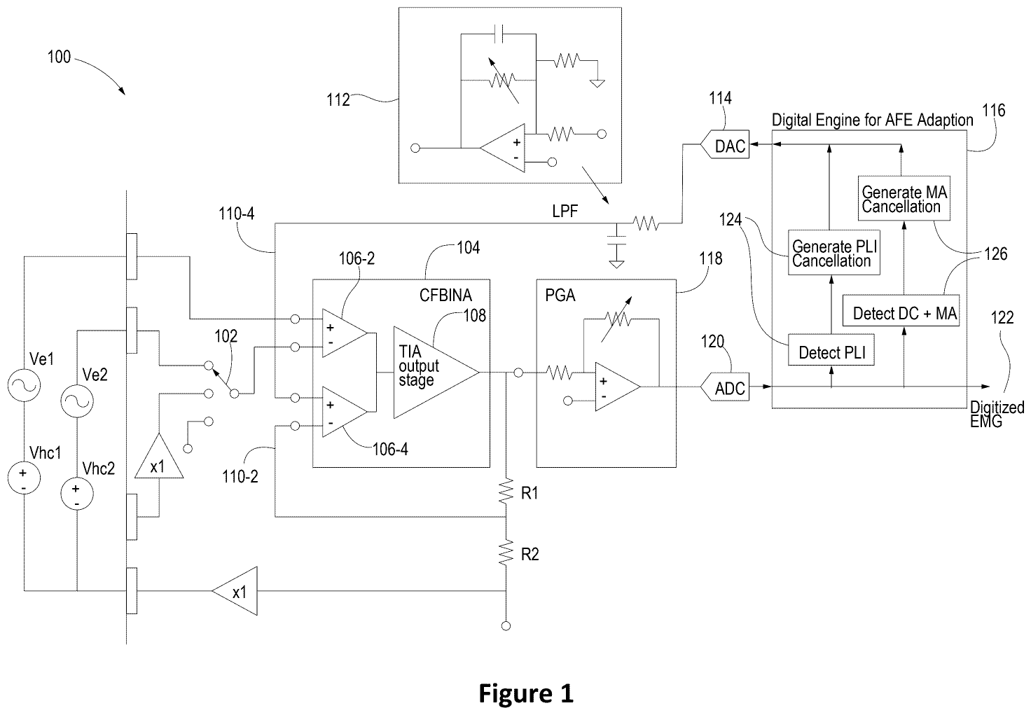

FIG. 1 is a schematic diagram of an example adaptive analog frontend (AFE) architecture 100, according to some embodiments. The diagram shows potential connections 102 (e.g., a three-way switch) that enable configurations of Bipolar, PMP and MP. The analog signal path is shown single ended after an electrode interface for the sake of illustration. The architecture may be implemented using discrete off-the-shelf components. The analog signal chain includes an instrumentation amplifier 104 (e.g., a current-feedback instrumentation amplifier (CFBINA)) with two input stages in the form of trans-conductors (gm stages) 106-2 and 106-4. Feedback is closed in the current domain past the input stages, where the input and feedback current signals are subtracted and fed to a transimpedance TIA output stage 108. The input stage dedicated to feedback receives two inputs, one input 110-2 from a feedback network (R2 and R1) and one input 110-4 from a low-pass filtered output 112 of a digital-to-analog converter (DAC) 114, which is driven by digital algorithms within a digital AFE adaptation engine 116. The CFBINA's output then drives a programmable gain amplifier (PGA) 118 whose output drives the input of an analog-to-digital converter (ADC) 120 that digitizes the conditioned analog signal.

The ADC output, after adaptation and compensation for artifacts, contains the digitized EMG signal 122, which is the system output. The digital output from the ADC 120 feeds the adaptive algorithmic engine 116, which comprises two separate estimation and compensation subsystems. These subsystems include a PLI algorithm block 124 for PLI detection and cancellation, and a subsystem 126 for DC, baseline and MA detection and compensation.

### Example Analog Signal Path Architecture

In some embodiments, the adaptive AFE has an overall noise cancellation scheme arranged in a feedback format. The variations of baseline DC, slow and fast, as well as narrow band interferences, such as PLI, are generated in the digital engine and translated to an analog signal to be fed back directly to the input. The net effect can be seen as an adaptive (digitally assisted) servo compensation of the baseline as well as narrow-band filtering at the PLI and its harmonics.

An analog component that enables such feedback compensation is the instrumentation amplifier 104, which may be a conventional analog circuit. Noise cancellation at the input enables allocation of large gain to the instrumentation amplifier 104 as desired in precision readout systems to help relax the requirement on the following components (the PGA 118 and the ADC 120, in terms of noise and resolution). This furthermore helps relax the overall signal path's dynamic range as it only has to accommodate an EMG signal.

The choice of current feedback instrumentation amplifier provides the flexibility of maintaining input common-mode range for the main electrode interface and using a single DAC to drive the noise cancellation signal through the auxiliary input trans-conductor. There are furthermore other possibilities to mitigate the effect of large electrode offset at the input driving the input trans-conductor non-linearity. In principle, a 3-operational amplifier instrumentation amplifier can also be used for this purpose, however proper replication (and hence cancellation) of input common-mode across the gain resistor will not be possible with a single DAC and an ADC at the backend of the signal chain.

A general consideration in the design of the compensation path is the quantization noise of the feedback DAC, which will be referenced to the input and comparable to the input noise. This is one of the design parameters, which determines the DAC architecture as well as the reconstruction analog passive filter following it before interfacing the CFBINA's reference input.

### Example CFBINA

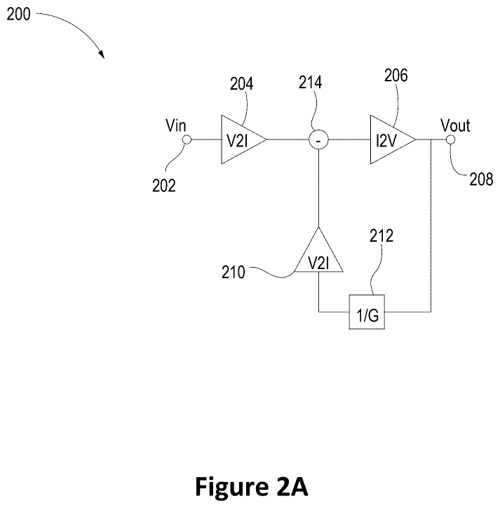

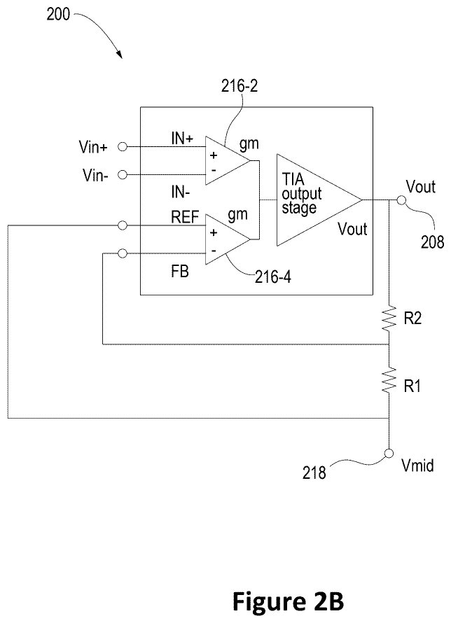

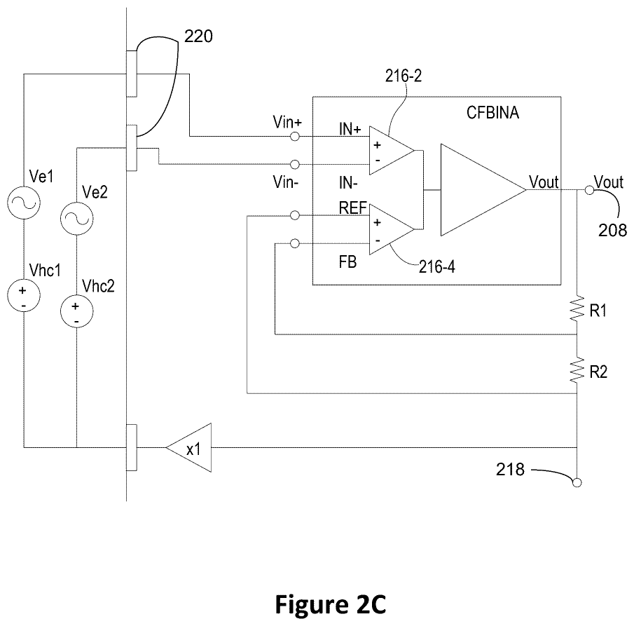

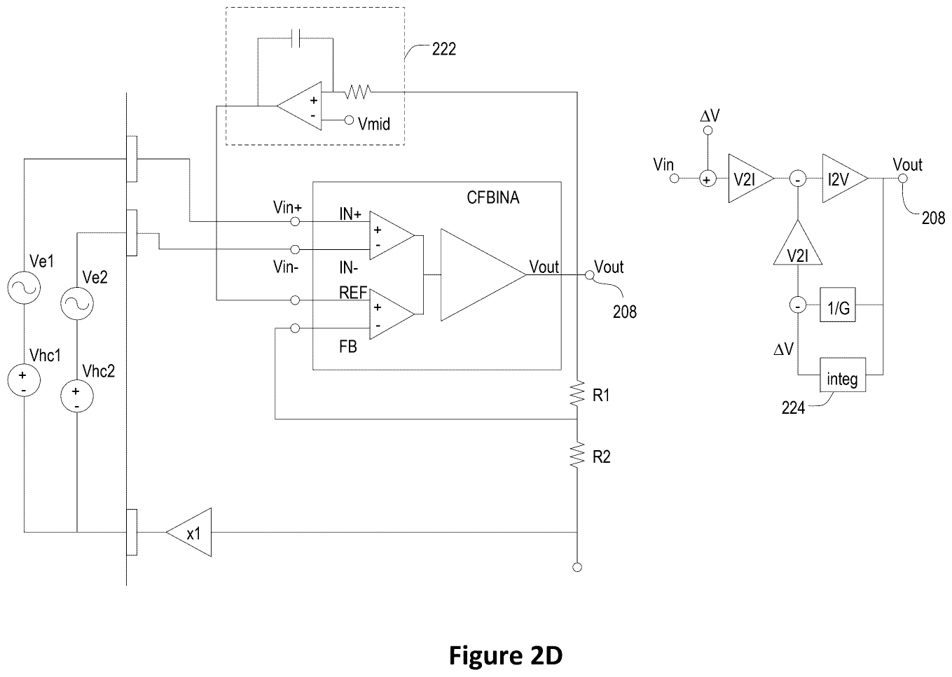

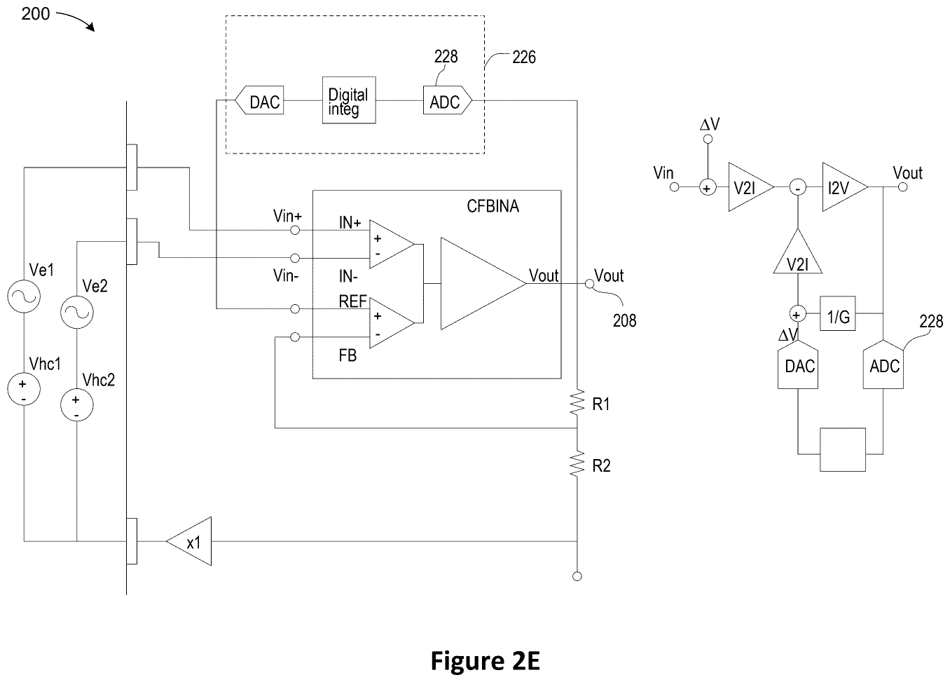

FIG. 2A is a schematic diagram of an example CFBINA 200, according to some embodiments. The CFBINA 200 is an example of the instrumentation amplifier 104 of the AFE architecture 100 described above. FIG. 2A is a simplified high-level block diagram of the CFBINA. The CFBINA interfaces the input signal 202 with an input voltage-to-current (V2I) converter 204, or a transconductance. This produces an output current that is proportional to the input signal. An auxiliary (and matching) V2I 210 or transconductance interfaces the feedback network that interfaces the divided version 212 of the output signal 208 through the feedback network. This produces an output current that is proportional to the feedback signal. The output currents of the two trans-conductors are subtracted post their connection node 214 and the error current is translated to an output stage, which is a transimpedance function (TIA) 206. With large open loop gain, the closed-loop gain of the amplifier is the inverse of the feedback factor G.

FIG. 2B is a detailed block diagram of the example CFBINA 200, according to some embodiments. Two trans-conductor stages gm (216-2 and 216-4) match and their outputs arc shorted, which results in subtraction of their currents with proper polarity. Combined with the output stage transconductance, they define the open loop gain of the amplifier, which when large results in a closed loop gain of 1+R2/R1. In the example shown, the output signal 208 is referenced to a Vmid reference voltage 218, which can be different from the input signal's common mode.

FIG. 2C is a block diagram of the example CFBINA 200 with connections to a bipolar electrode pair 220 for biopotential sensing, according to some embodiments. The connection is not limited to bipolar sensing and any pseudo-differential scheme, such as Pseudo-Monopolar (PMP) sensing or Monopolar (MP) sensing may be used, e.g., by connecting one of input terminals of the input gm to a reference. The electrode offset of half-cell potential can be +/−300 mV and with a 1.8 V system, even a small gain of times 5 [V/V] can easily saturate the signal path.

FIG. 2D is a schematic diagram of the example CFBINA 200 including an analog servo loop 222, according to some embodiments. The analog servo loop uses an integrator 224 that directly senses the DC at the CFBINA output and drives the Vref input to the correct value that cancels out the electrode offset. The low-pass function in feedback can be seen as a parallel path to the resistive feedback network of the CFBINA. Its effect in the forward frequency response results in a bandpass function. The unity gain frequency of the integrator loop determined by 1/RintCint and the overall CFBINA closed loop gain determines the high pass frequency corner of the overall signal path. There are two potential challenges with this architecture: (i) the required 10˜ 15 Hz high pass corner requires a very low unity gain frequency for the integrator that translates to large resistor/capacitors, and (ii) the fixed unity gain frequency (high-pass corner frequency) creates an effective bandpass function, however any motion artifacts with frequency content above that corner frequency end up not being filtered and get amplified by the CFBINA.

FIG. 2E is a schematic diagram of the example CFBINA 200 including a digital loop 226, according to some embodiments. The low pass filtering function can be implemented in the digital domain and the output of that digital filter can drive a DAC to generate an analog signal that drives the CFBINA's Vref. An ADC 228 digitizes the CFBINA's analog output but usually the ADC is already there in the backend of the signal chain following a second stage amplification usually implemented as a programmable gain amplifier or PGA (so there is no need to have an additional and dedicated ADC as implied by FIG. 2E). A benefit of this alternative is the freedom in digital domain to create very large time constants (the integrator unity gain frequency no longer need large resistors and capacitors) and in addition, the application of adaptive monitoring techniques, such as rate of change monitor can be applied to dynamically adjust the bandwidth integrator/low-pass filter path in the feedback. This results in an adaptive high-pass function. The large magnitude and fast changes of the input signal can trigger an instant bandwidth adjustment to track and cancel their effect at the input. This is further described below in reference to the motion artifact tracking algorithm.

Example specifications of the CFBINA are shown below:

| Specification | Typical values | |
|---|---|---|
| Unity gain bandwidth | 200 | kHz |
| Input voltage noise | 25 | nV/rt(Hz) |
| DC gain | 50 | dB |
| Rms noise in 1 kHz | 2u | Vrms |
| DC closed loop gain | ~50 to 100 (R2, R1 of 50 kohm, | |
| | 1 kohm or 100 kohm/1 kohm) | |
| Closed loop bandwidth | 1 | kHz |
| (low-pass corner) | | |
| CMRR @ 60 Hz | 100 | dB |
| Typical high-pass corner | 10~20 | Hz |

| | |
|---|---|
| Supply, Vmid | 1.8 V, 0.9 V |

In some embodiments, parameters are adjusted to those of the OTS parts. In some embodiments, the parameters can be further optimized for better signal-to-noise ratio (SNR) and power consumption performance.

In some embodiments, a three operational amplifier (op-amp) instrumentation amplifier (INA) is used as an alternative to the current-feedback instrumentation amplifier. Such three op-amp INA includes a node to inject the analog compensation signal. One place to inject differential signals is the mid-point of the two input op-amp Rin resistor (e.g., inject a direct current (DC) servo loop's current into that node). In a three op-amp INA, resistor is floating as far as input common-mode is concerned. The two input op-amp reflects both the common-mode and differential components of input signal across it. The differential component gets amplified while the common-mode part appears at the output of those two op-amps and rejected by matching network of the third op-amp. Some embodiments perform analog compensation in three op-amp INA. For digital compensation, a three op-amp INA cannot have separate feedback and input common-mode levels. The input common mode is present at the output of the first two op-amps. It is rejected after the third op-amp. This means injecting a differential compensation signal to the mid-point of the input resistor will require the common mode signal of the input also to be generated. This is possible if two separate DACs are used, each carrying a common and differential signal component. However, to close the common-mode signal loop in digital domain, the common-mode signal also has to be digitized at the output of the INA's first two op-amps. Accordingly, in some embodiments, two separate ADC's observe the first two op-amps of the three op-amp INA. The ADCs are used in addition to the ADC at the output of the signal chain.

A CFBINA disentangles the common-mode of the input and output hence the feedback path can follow a different common-mode level (thanks to two parallel trans-conductors one dedicated to input and one to the output). This enables ignoring the input common-mode level in the whole signal chain related to artifact estimation and compensation. Some embodiments estimate the artifacts that show up differentially at the digital output of the ADC present at the end of the signal path (no need for additional ADCs). Some embodiments compose the input referred compensation signal single ended and apply it in addition to the CFBINA's feedback signal to the auxiliary trans-conductor of the feedback path, irrespective of the input common mode. This means that the only additional analog components required is a single-ended DAC.

### Example Programmable Gain Amplifier (PGA)

The programmable gain amplifier or PGA 118 can be any type of amplifier with programmable gain. This block can be skipped, for example to improve energy efficiency. The PGA can be used to set the overall channel gain (from the electrode to ADC input) to numbers between ×500 [V/V] and ×1000 [V/V] (a 1 mV amplitude signal to be amplified to 1 V for an ADC with 1 V dynamic range will require such level of gain). Placing all the gain into one amplifier stage can significantly increase the requirements on its unity gain bandwidth. For example, 1 kHz closed loop bandwidth with a closed loop gain of 60 dB will require at least 1 MHz unity gain bandwidth and this might incur power consumption penalties that could be better optimized by distributing gain.

Some embodiments use a PGA that is assumed to have a gain of ˜×10 [V/V]. A bandwidth of 1 kHz can be assumed. In some embodiments, the PGA's gain is not varied. In some embodiments, gain adaptation is applied (e.g., after fast motion artifact recovery).

### Example ADC

In some embodiments, a goal is upfront cancellation of PLI, DC electrode offset, motion artifact, and/or baseline wander. Accordingly, the ADC 120 comprises a moderate resolution ADC. In some such embodiments, the dynamic range of the signal path will be mainly dedicated to the EMG signal, rather than accommodating the large artifacts.

Some embodiments use a 12-bit ADC architecture with a full-scale of the 1.8 V supply, meaning an LSB size of approximately 440 microvolts (uV). Referring this to the electrode through a channel gain of ×500 [V/V] corresponds to a resolution of 0.88 uV, which is lower than the input referred AFE noise of 1.5-2 microvolts root-mean-square (uVrms). A gain of ×500 within a 1.8 V system still has plenty of margin assuming even a 2.5 mV amplitude EMG signal. The larger the frontend gain, the less the effect of ADC quantization noise when referred to the electrode, so a 12-bit ADC is likely within the range of acceptable resolution. Assuming artifact (noise) cancellation at the frontend, ADC resolution beyond this, at the Nyquist rate of the input signal, will not result in any additional SNR but only incurs additional power consumption.

In some embodiments, the ADC sample rate is 20 kHz, which provides ×20 oversampling to the signal bandwidth and ensures minimal loop delay in the compensation digital algorithms running in a feedback compensation scheme. This level of performance for the ADC can enable the adoption of energy efficient architectures, such as Successive Approximation Register Analog-to-Digital Converters (SAR ADCs).

### Example DAC

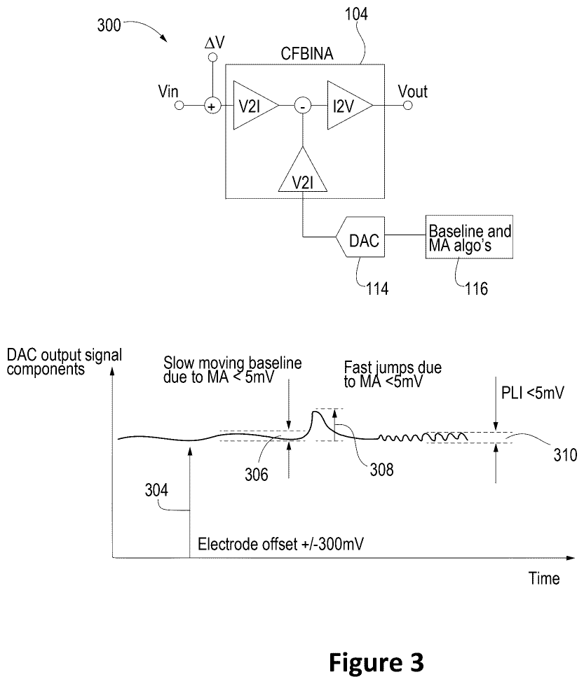

FIG. 3 is a schematic diagram of an example circuit 300 with the DAC 114, according to some embodiments. In some embodiments, the DAC 114 receives the digital artifact compensation signals from the digital algorithm 116 and generates an analog signal that drives the Vref input of the instrumentation amplifier 104 to counteract the effect of DC offset, baseline wander, MA and PLI. The resolution and hence quantization noise produced by the DAC 114 is directly input referred. So the DAC 114 does not have the advantage that the ADC 120 has, which is being preceded by the gain of instrumentation amplifier 104 and the PGA 118. This means that the DAC 114 is likely to require higher resolution to not contribute to the input referred noise.

One advantage of the input referred artifact compensation scheme described herein is that the full-scale dynamic range of the DAC 114 need only cover the electrode offset for the largest (e.g., +/−300 mV) 304, slow baseline movements due to motion artifact (<5 mV) 306, fast transients of MA (e.g., <5 mV) 308, and the PLI (e.g., <5 mV) 310. This is likely a total dynamic range of 1 V assuming a lot of margin. This amounts to a total of 1 uVrms quantization noise contribution. Accordingly, a 20 bit DAC is likely needed.

When considering the compensation signals, some embodiments consider the effect of accuracy in each signal. For instance, the electrode offset of 300 mV can be compensated with a few hundred micro-volts of error and the residual error will not take up the dynamic range of the system. The compensation of the fast transient MA also can tolerate some quantization error as the main goal is to track it sufficiently well to prevent saturation. On the other hand, other components like slow baseline tracking or the PLI tracking and compensation require sufficient resolution. In case of PLI, quantization error impacts the phase and frequency lock of specially the higher harmonics within the tracking scheme. A benefit is that the demanding portions of the signal will probably take up only a small portion of that 1 V dynamic range, for a likely total of 10 mV, which usually needs higher resolution than the rest.

This specific characteristic is a motivation for a hybrid DAC. The hybrid DAC is more efficient compared to a 20 bit DAC. In some embodiments, the hybrid DAC is a regular moderate DAC, e.g., a 12 bit DAC where a select number of its most significant bits (MSB), e.g., 9 to 10 bits cover the larger and less sensitive portions of the dynamic range (e.g., for large electrode offset compensation) and the rest of its lower significant bits (LSB), e.g., 2 to 3 bits, can be driven by means of a delta-sigma modulator to implement noise shaping and oversampling to effectively increase their resolution. The hybrid DAC may be viewed as the reverse of a zoom ADC.

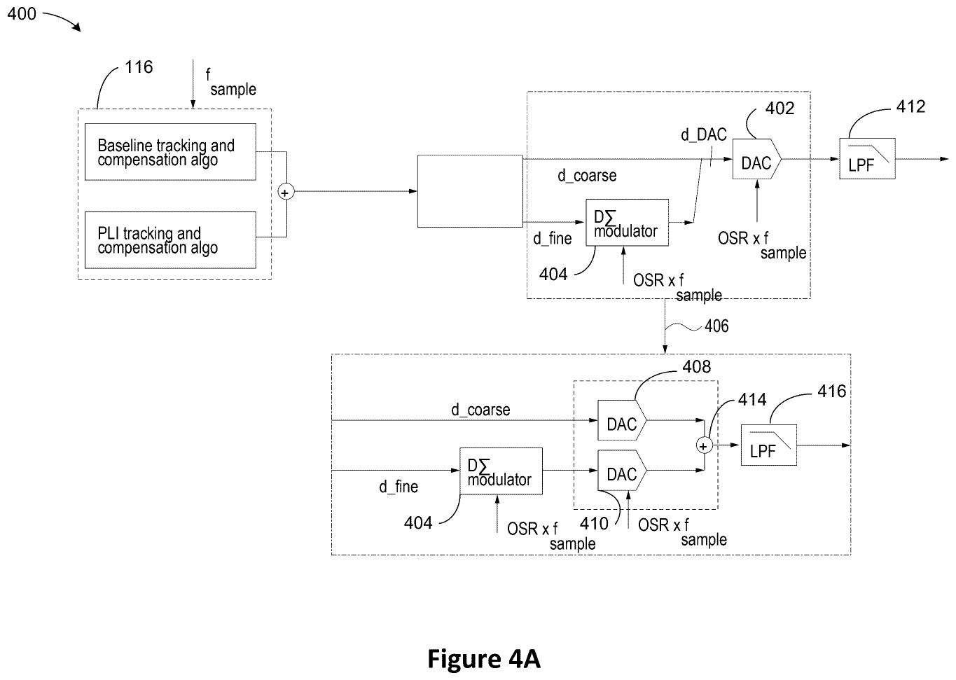

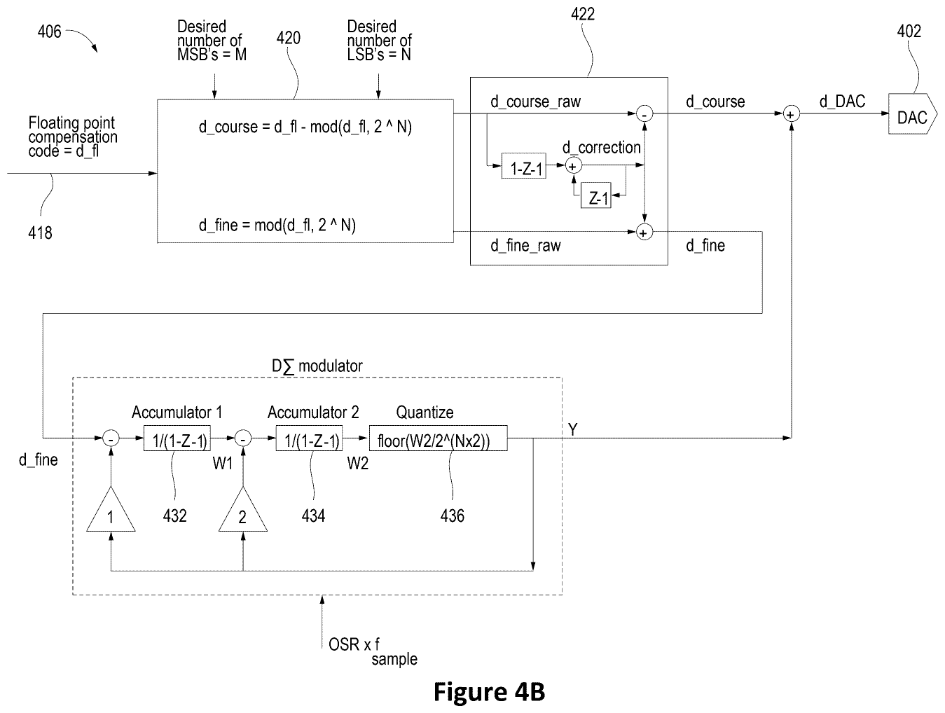

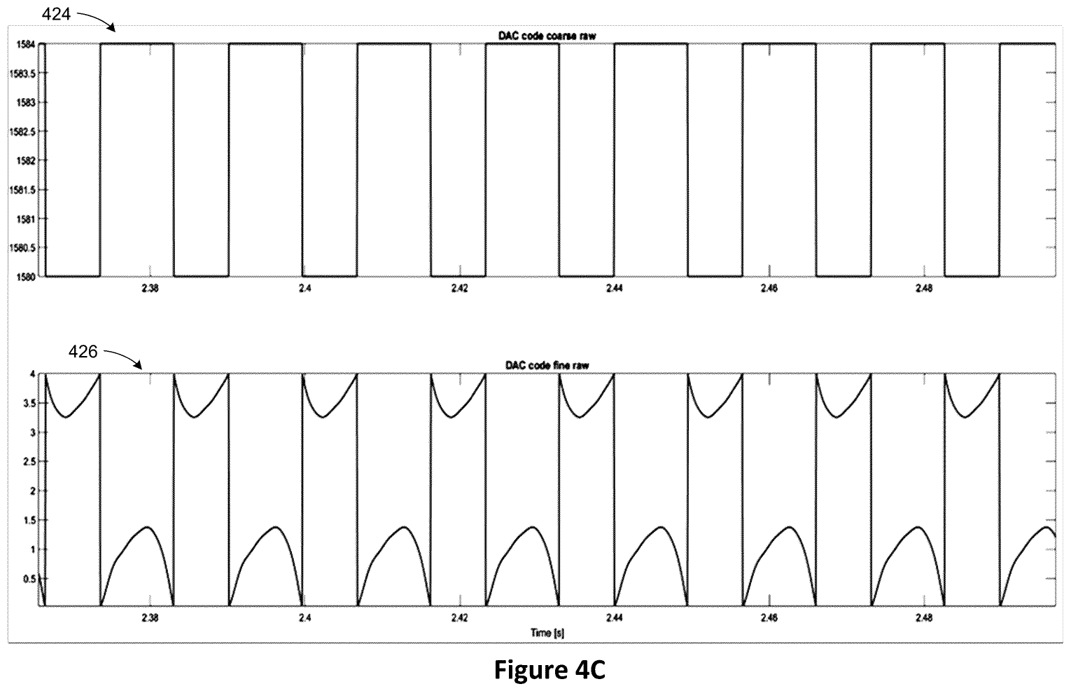

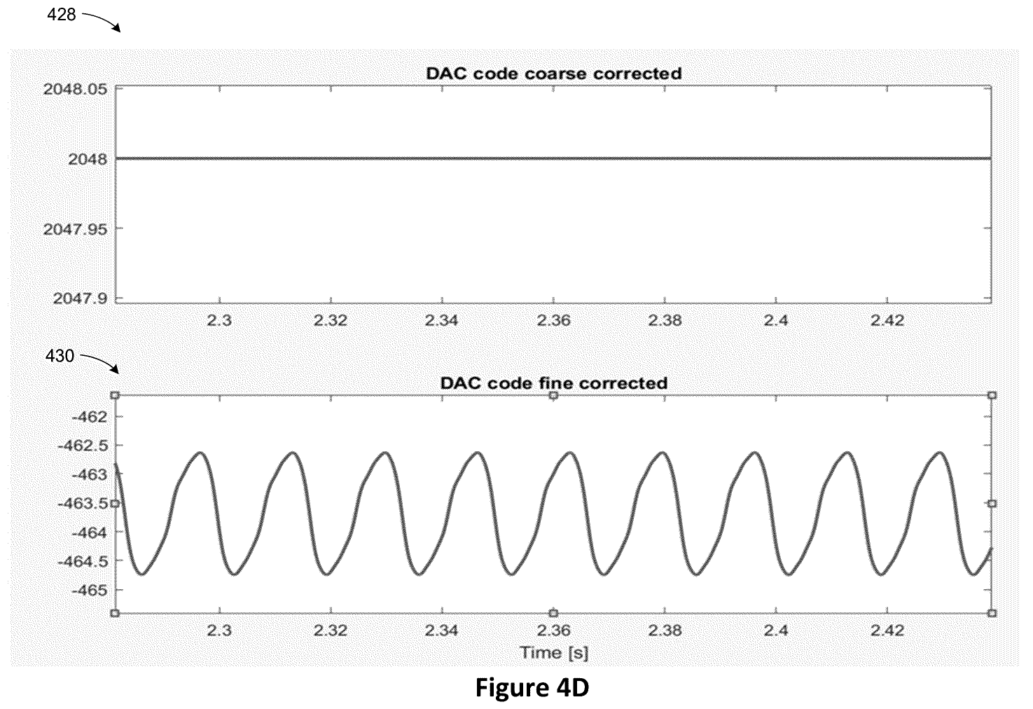

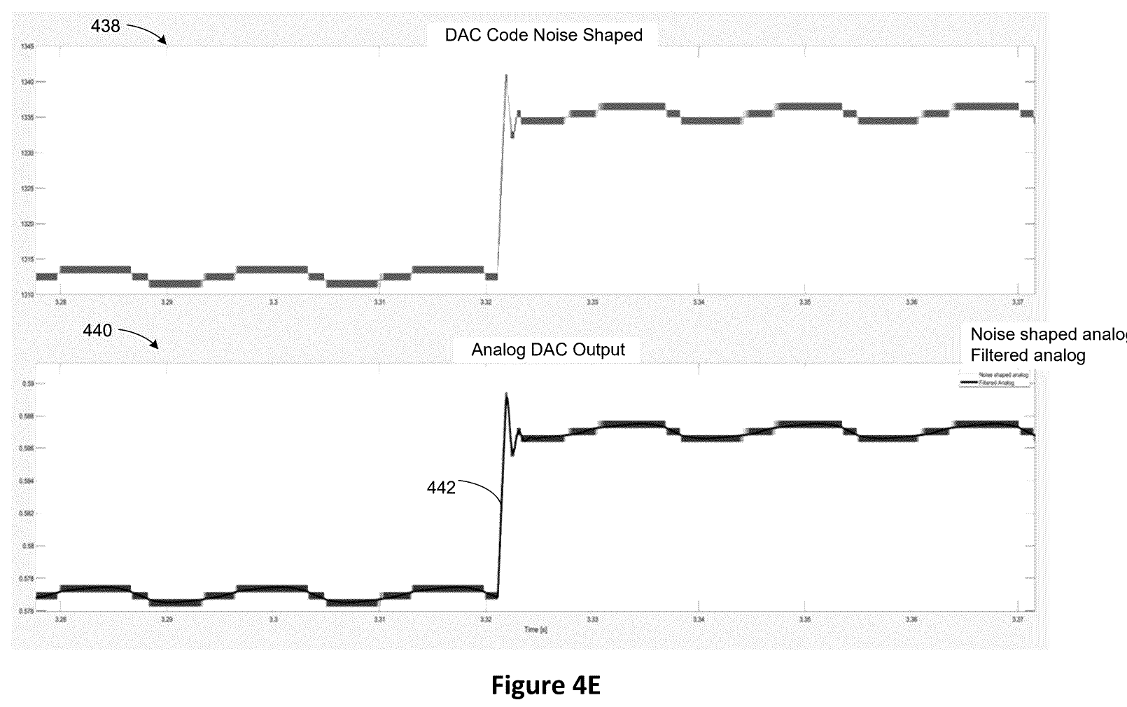

FIG. 4A is a block diagram of an example system 400 using a hybrid DAC, according to some embodiments. Some embodiments include a DAC 402 that is configured to run at the oversampled sample rate (OSR) of a delta sigma modulator 404 (OSR×f_sample). The coarse MSB codes (d_coarse) are updated at the lower sample rate of the ADC and adaptation algorithm 116, however the LSBs are updated at the oversample rate of the delta sigma modulator 404. The DAC can be configured to update its analog output at OSR×f_sample. In some embodiments, the DAC can have sample rates up to 1 MHz. For instance, a delta sigma modulator running at 500 kHz provides plenty of oversampling ratio for the bandwidth of PLI signals and is still reasonable to be paired with a DAC.

The subset 406 in the block diagram shows an embodiment of the hybrid DAC where a current steering DAC 408 is arranged to have its MSB segments driven by the coarse code at the lower sample rate f_sample and the few noise shaped LSB's switched at the oversample rate of OSR×f_sample. The output currents of both Nyquist rate coarse DAC 408 and delta sigma modulated fine DACs 410 are summed up 414 in the current domain and filtered through an output stage transconductance (combination 416 of current-to-voltage and filter in one stage). With the DAC output in the voltage domain, a passive LPF 412 is sufficient for reconstruction filtering.

FIG. 4B is a detailed view of the hybrid DAC shown in the subset 406, according to some embodiments. Floating point output 418 of the MA tracking and PLI tracking algorithms 116 are broken down (420) into a combination of M bits of coarse and N bits of fine codes. M and N can be any desired combination, e.g., 10 and 2, respectively, if a 12 bit DAC hardware is concerned. For example, the fine code is extracted by calculating the modulus of division of the floating point algorithm output number by 2\hat{ }N (the integer maximum value of the fine code). The coarse code is the difference between the floating point algorithm output number and the fine code. The fine code supplied to the delta sigma modulator is in floating point. Truncation occurs after the delta sigma's quantizer.

With the coarse and fine division of the algorithm output, an AC signal, such as PLI compensation superimposed on a baseline signal, such as electrode offset, can drive the coarse code back and forth between two adjacent segments covered by the MSBs (e.g., as if the coarse codes have a +/−1 code variation modulated by the AC signals). This will reflect as a roll over effect on the fine code. FIG. 4C shows example graph plots 424 and 426, corresponding to raw DAC code coarse and raw DAC code fine, respectively, according to some embodiments. The sum of coarse and fine codes still results in the correct baseline and PLI comp AC signal. However, the quick roll over jumps in input code is not suitable for the delta-sigma modulator. A delta sigma modulator cannot operate with broad band input signals and it can process signals in its bandwidth. If such roll-over jumps are fed to the delta-sigma modulator 404, it results in glitches at the output due to its limited bandwidth that when combined with coarse DAC output will still be present in the analog output signal.

Referring back to FIG. 4B, accordingly, in some embodiments, a roll-over correction mechanism 422 identifies such back and forth roll-over (e.g., due to coarse code jumping back and forth) between two segments using a differentiator and an integrator. When a jump occurs in the coarse code by +1 or −1 LSB, this value is extracted through differentiation. This mechanism is similar to a dirac delta that shifts the following integrator until the next jump brings the integrator back and so on and so forth. The integrator output will then represent a correction signal that is subtracted from the coarse code and added to the fine code to ensure steady values for both. FIG. 4D shows graph plots 428 and 430 corresponding to corrected DAC code coarse and corrected DAC code fine, respectively, according to some embodiments.

Referring back to FIG. 4B, the delta sigma modular 404 includes an accumulator 432 followed by an accumulator 434, which is followed by a quantizer 436, according to some embodiments. In some embodiments, the digital delta sigma modulator 404 has larger dynamic range by the size of one coarse segment, which means twice the range covered by the desired N number of LSBs. In this way, the dynamic range of the delta sigma modulator's quantizer 436 is increased to 2 times N.

FIG. 4E shows example graph plots 438 and 440, corresponding to final noise shaped DAC code and pre low pass filtered analog hybrid DAC output (solid line), respectively, according to some embodiments. The thin line 442 in the graph plot 440 corresponds to the final filtered analog output (post reconstruction low-pass filter after the DAC). This snapshot captures a moment of reacting quickly to an offset jump (similar to electrode lift-off) while compensating for the coupled input PLI.

### Example Low-Pass Filter (LPF) Post-DAC

In some embodiments, the post DAC LPF (the LPF 412, FIG. 4A) is configured to smooth out fast switching noise of the delta sigma modulation 404. The LPF bandwidth cannot be too low as it will affect the overall compensation loop stability. Some embodiments use a corner frequency of 0.25 times fs_dig (the sample rate of ADC 120 and the algorithm 116). For a 20 kHz sample rate, this corresponds to a 5 kHz filter. First order filters or second order filters may be used. The loop has sufficient stability for both types of filters. In terms of effective removal of delta sigma modulated noise, it is important to see it from the point of view of quantization noise within the effective bandwidth of the AFE. Because the delta sigma modulated noise will be fed back to the AFE, any noise that is outside the effective bandwidth of the AFE will be filtered by the AFE. Hence, the LPF's requirement can be relaxed on the noise that is outside the AFE's effective bandwidth.

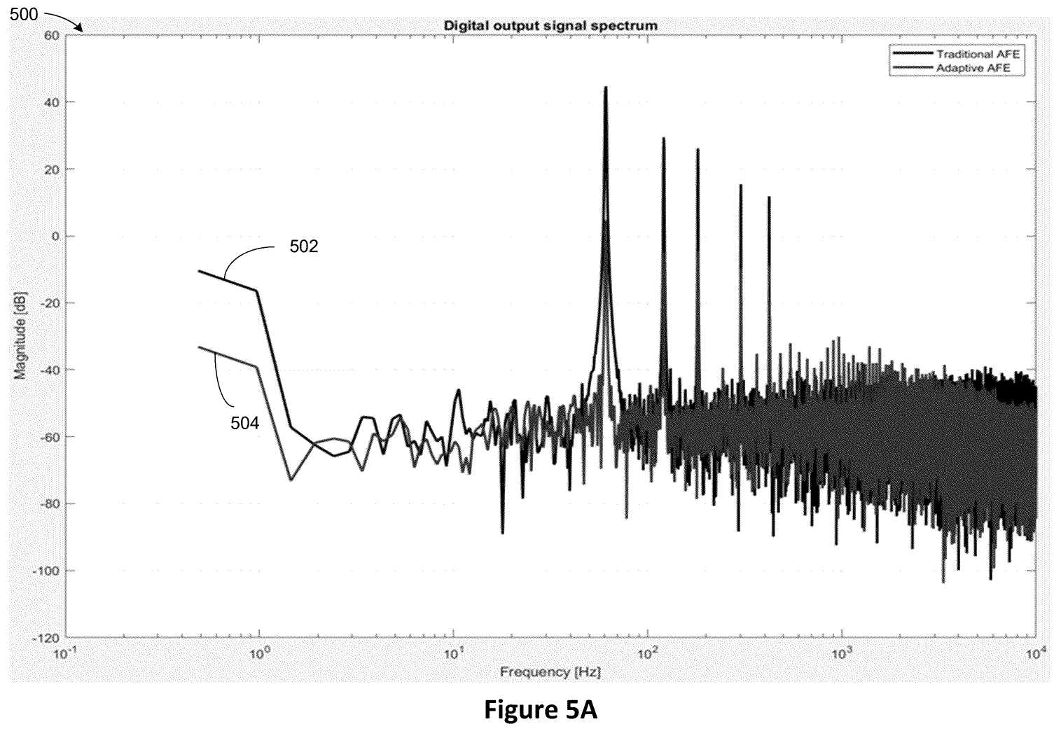

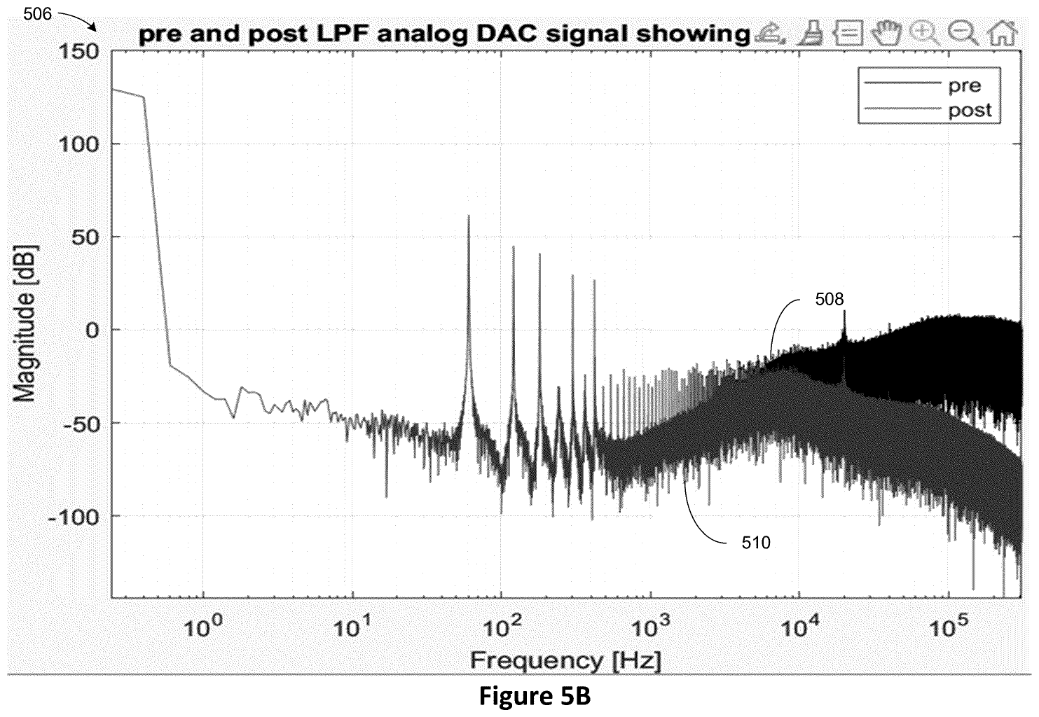

The combination of oversampling ratio and order of the delta sigma modulator can be chosen in such a way that the output referred noise of DAC without any LPF remains below the target input referred noise of less than 2 uVrms within a noise bandwidth of 1 kHz. This noise bandwidth is an integration noise bandwidth and not a physical filter's bandwidth. The effective physical filtering is performed by the AFE's low-pass function with a corner of 1 kHz. So, as long as the quantization noise is suppressed sufficiently within the 1 kHz bandwidth, the out of band noise will be effectively suppressed by the forward path provided by CFBINA and PGA. With a first order LPF after the DAC combined with AFE, an effective third order filtering at the overall system output is achieved. FIG. 5A shows a graph plot 500 comparing digital output signal spectra (magnitude in dB versus frequency in Hz) for traditional analog frontend 502 versus adaptive analog frontend 504, according to some embodiments. As shown, the in-band noise floors remain the same for both cases. FIG. 5B shows a graph plot 506 comparing pre-LPF analog DAC signal 508 and post-LPF analog DAC signal 510, according to some embodiments. The pre- and post-LPF spectra in the feedback path show that upmodulated noise is removed only partially by the LPF only after 5 kHz, however the in-band noise (within 1 kHz) is sufficiently flat.

### Example Motion Artifact Algorithm

In some embodiments, the motion artifact (MA) algorithm 126 (FIG. 1) is based on the characteristics of large magnitude and fast rising baseline jumps (e.g., jumps seen due to loose band in existing data). In some embodiments, the algorithm does not rely on a parallel sensing modality, such as inertial sensing or bio-Z measurement, which correlates with MA, but rather directly looks at the incoming EMG signal's rate of change. Some embodiments use an adaptive bandwidth tracking algorithm that reacts to rate of change of the signal, once larger than programmable adaptive thresholds. When triggered, the algorithm allows fast tracking of the baseline compensation path in the feedback. The algorithm is refined using limited available data and reasonably responds to fast changing run away situations. These are situations in which a fixed high-pass corner (e.g., AC coupling or fixed bandwidth analog servo loops) does not react.

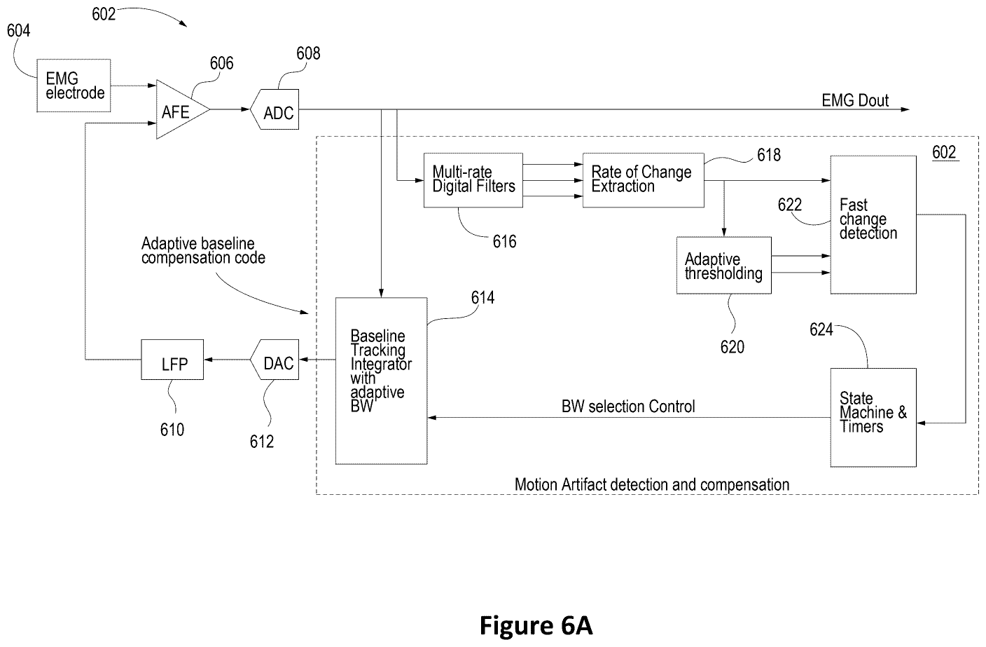

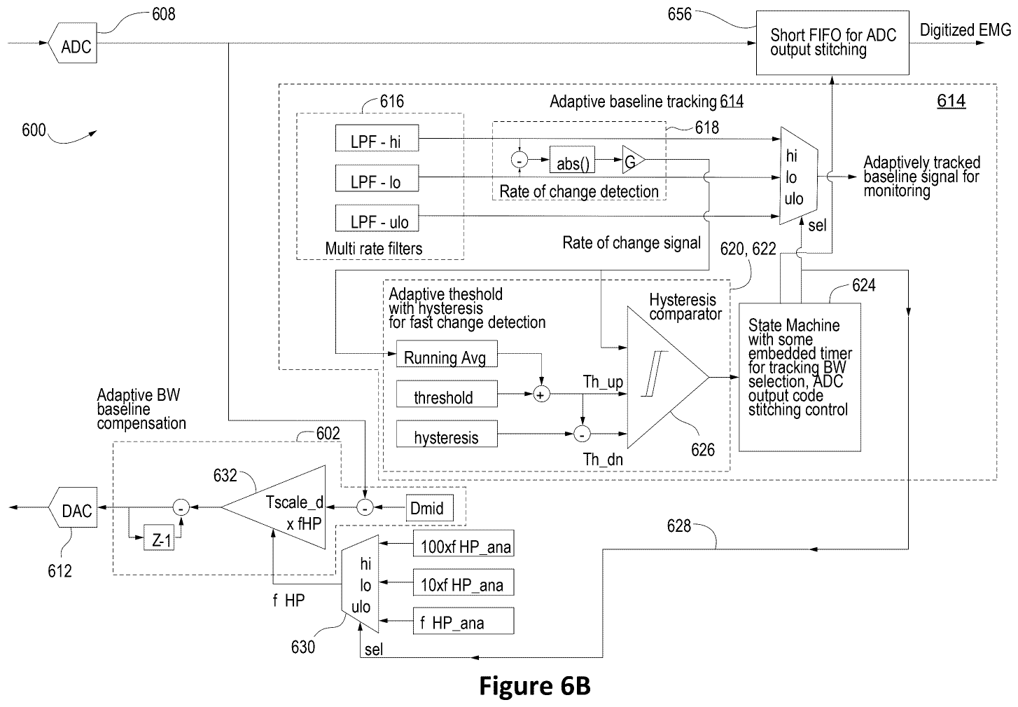

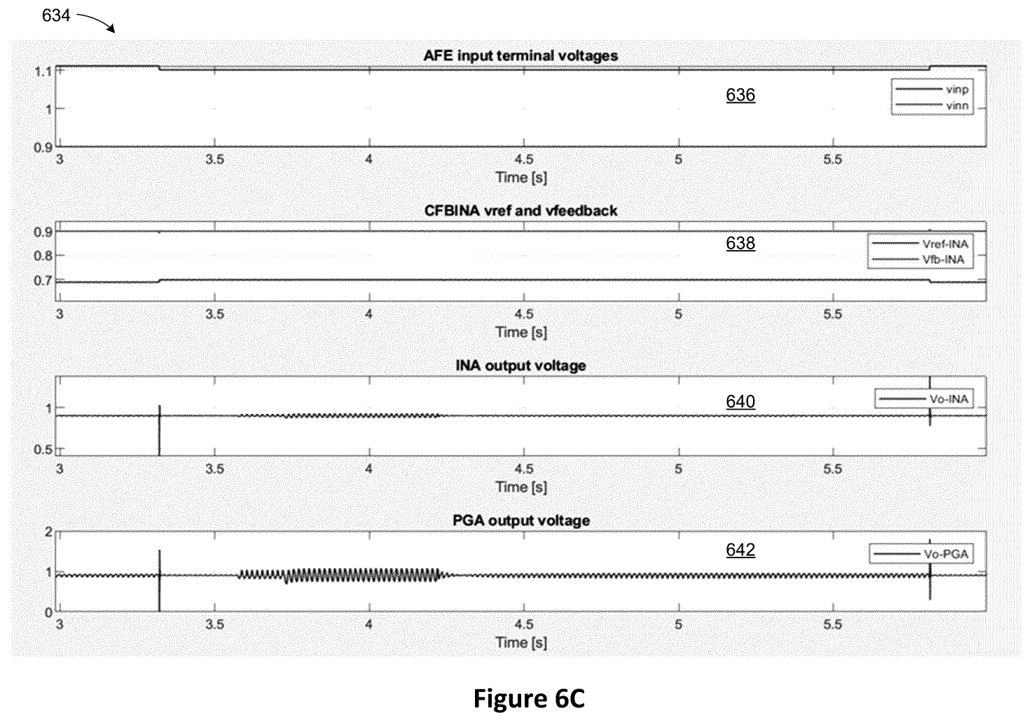

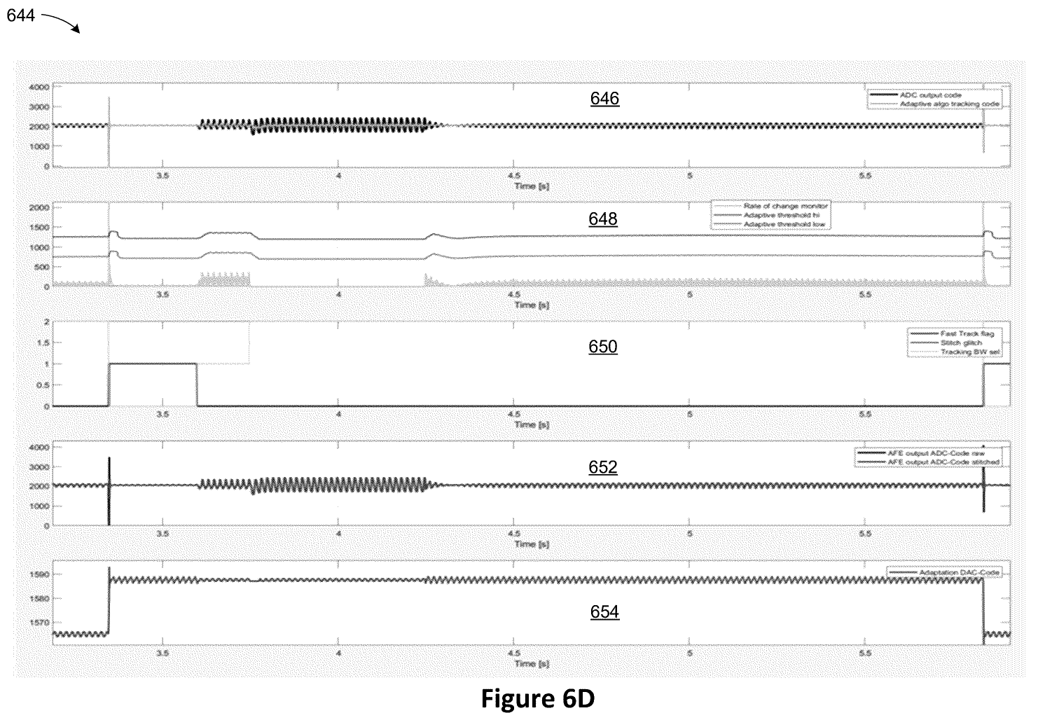

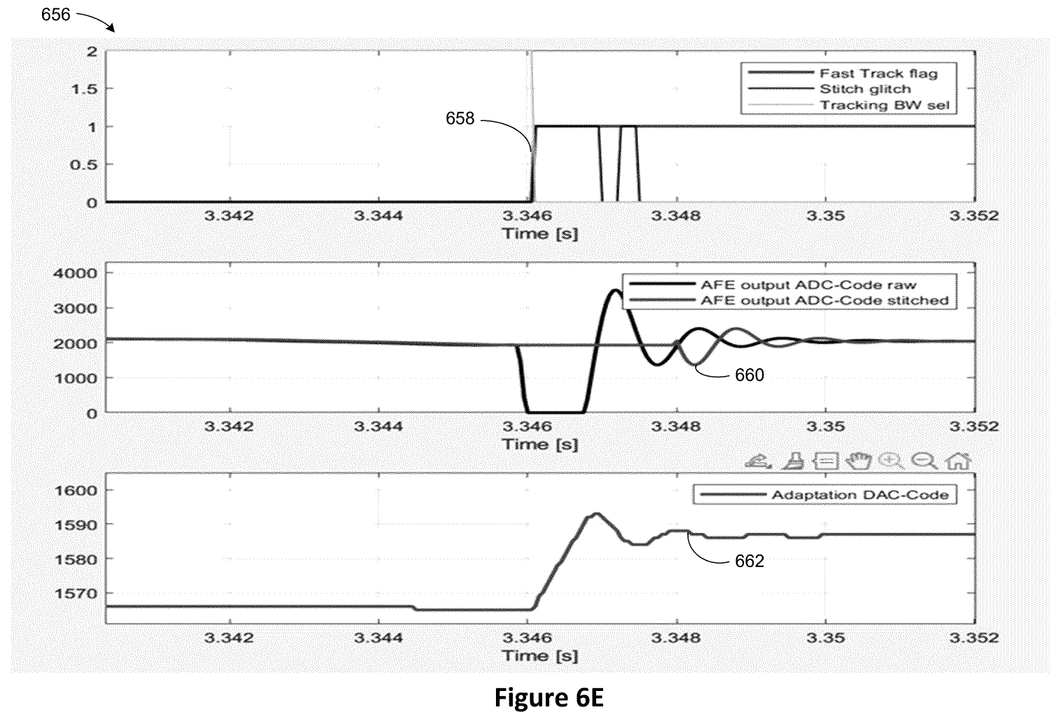

FIG. 6A is a block diagram of an example system 600 that includes an MA algorithm 602, according to some embodiments. The system includes an AFE 606 coupled to an EMG electrode 604. The AFE is coupled to an ADC 608 and compensation path DAC 612 and LPF 610. The ADC output passes a bank of multi rate filters 616 to examine the signal content in different bands. The outputs of the filters 616 are monitored by a rate of change extraction block 618 whose output signal is compared with adaptive hysteresis thresholds 620 to detect if the rate of change exceeds the thresholds. Once a fast runaway baseline situation larger than the threshold is detected (622), a block composed of state machine and timers 624 adjusts the bandwidth control knob of a baseline tracking integrator 614. The integrator 614 is the baseline compensation loop's memory that locks to the DC electrode offset. During stable situations, the integrator 614 passes the less than 20 Hz baseline movements together with the DC to the compensation path DAC 612. Once runaway fast liftoff events occur, its bandwidth is opened up to allow the fast transition to also pass the DAC. Once the runaway situation passes (governed by hysteresis thresholds, state machine and timers), the compensation integrator bandwidth is gradually reduced back to the stable level. In some embodiments, parameters in the blocks (e.g., filter rates, thresholds, adaptiveness of thresholds, timers, gains) are programmable, so they can be adjusted if needed in order to provide flexibility.

FIG. 6B is a block diagram showing a detailed view of the example system 600, according to some embodiments. The system comprises adaptive baseline tracking 614 and adaptive bandwidth baseline compensation 602. The former determines if a large and fast event has occurred and if and how the bandwidth of the latter, which is constantly tracking the DC and slow moving baseline, should be adjusted to track those. A large and fast event is an event that makes a signal acquisition system susceptible to saturation. Suppose the entire system has a gain of 300 with 1.8 V dynamic range. When the input has a sudden change of 3 mV or larger in around one millisecond, conventional noise cancellation mechanism will likely not work fast enough and the system is susceptible to saturation.

### Example Adaptive Baseline Tracking

In some embodiments, the adaptive baseline tracking 614 receives the ADC output signal and runs it through three parallel running multi-rate filters 616. The bandwidth of these filters can be programmed digitally, if needed. In some embodiments, each filter is a second order Butterworth transfer function with bandwidths of high, low, and ultra-low. In some embodiments, these filters are set at 2 kHz, 50 Hz and 20 Hz, respectively. The rate of change detection (e.g., the operation in change extraction block 618) subtracts the high and low filter outputs, calculates the absolute value of that difference, and gains up by a digitally programmable gain. This signal is the rate of change signal that is passed to an adaptive thresholding function 620 for fast change detection 622. The detection is done by means of a hysteresis comparator 626, which has an upper crossing th_up and lower crossing th_dn threshold input. These two thresholds are calculated by the digitally programmable threshold and hysteresis values by adding and subtracting them to and from the output of a running average filter that filters the rate of change signal, respectively. To illustrate, threshold is used as an example herein. By default, threshold has a fixed value and the signal and the threshold are compared to determine the part of the algorithm that needs to be to run. A goal of the threshold is to monitor the sudden change of the signal instead of accumulated change. Accordingly, the threshold value cannot be fixed but has to be adaptive. To make this threshold value adaptive, the threshold value follows the average value of the signal by adding or subtracting the original fixed threshold with the average value of the signal, according to some embodiments. The hysteresis value works similarly. This allows the thresholds to slowly track the average of noise and reduce the effect of false triggering. In some embodiments, the length of the running average filter is also programmable by a separate parameter in the code of the model. The hysteresis comparator output is a fast tracking flag signal that goes up when the th_up is crossed upwards and goes back down when the th_dn is crossed downwards (hysteresis behavior). The flag triggers a state machine with embedded timers. In some embodiments, the timers are programmable.

In some embodiments, the working of the state machine is as follows: if rate of change goes higher than upper threshold, reset the fast track monitoring timer and set the fast track flag high. If rate of change drops below the lower threshold, remove fast track flag only if fast tracker time kept by timer has been elapsed, and if timer time has not elapsed, keep updating the counter and keep the fast track flag high. This ensures that the system stays in a fast track mode by the amount of time defined by the timer. Once the fast track timer elapses, the system transitions to gradually settle to an ultra low bandwidth baseline tracking by transitioning through a mid level slow tracking bandwidth. In some embodiments, the amount of time spent in this mode is governed by a separate programmable timer. After that time elapses, the system goes to the slowest tracking bandwidth to mimic the high pass corner fHP_ana of an analog servo loop. If any fast moving event occurs during any of these phases, the flag is immediately raised and state machine rests back to fast tracking mode, according to some embodiments.

### Example Adaptive BW Baseline Compensation

In some embodiments, the result of the adaptive tracking block 614 is a controlled selection signal 628 going to a multiplexer 630 that feeds a compensation integrator 632 in the adaptive bandwidth baseline compensation 602 with the adaptive bandwidth. These can also be programmable; some embodiments use 100×fHP_ana, 10×fHP_ana and fHP_ana for fast, slow, and ultra slow tracking bandwidths, respectively. Some embodiments use more intermediate points than the three described above.

The motion artifact tracking algorithm (e.g., the adaptive baseline tracking 614) runs at a same sampling rate as the ADC 608, which can be imagined to have a lower bound of 8 kilo Samples per second (kSps) and may have an upper bound of approximately 20 kSps. A maximum bit depth of 16 bits can be imagined for the incoming data. One or more programmable knobs (e.g., static register, programmable) may be used to provide flexibility in the algorithm functions.

FIG. 6C shows graph plots for example operation 634 of the motion artifact algorithm, according to some embodiments. Graph plot 636 corresponds to AFE input terminal voltages over time, shows various analog nodes in the signal path. Graph plot 638 corresponds to CFBINA vref and vfeedback over time. Graph plot 640 corresponds to INA output voltage over time. And graph plot 642 corresponds to PGA output voltage. This simulation also included PLI (hence the 50 Hz signal present). PLI is described below. A 300 mV DC offset is applied and at a given time a 10 mV step like offset jump is applied to the inputs.

FIG. 6D shows graph plots for example operation 644 of the MA algorithm, according to some embodiments. The first row 646 is the ADC output code and the adaptive tracking's monitor signal. The second row 648 shows the rate of change monitor signal and the two high and low adaptive thresholds (th_up and th_dn). Once the step is applied, the signal in the bottom passes the upper threshold. The third row 650 shows the internal state machine flags and bandwidth selection controls. The row 652 is the ADC code with and without the stitching mechanism. It shows how the stitching mechanism de-glitch the ADC output. The row 654 shows the DAC output which confirms that the digital feedback loop will cancel the glitch eventually. In some embodiments, when the first fast rising is detected, the state machine switches to the fastest tracking bandwidth and the DAC output goes to track the input. The state machine stays in the fastest tracking (e.g., tracking bandwidth selection is set to 0) for the duration of the timer. When there are no further events, the state machine switches to the intermediate bandwidth (e.g., tracking bandwidth selection is set to 1), and once that timer elapses, the state machine goes to slow tracking (e.g., tracking bandwidth selection is set to 2). In some embodiments, when the 10 mV offset at the input is removed, another fast running event is detected and the DAC goes back to the DC offset of 300 mV.

### Example ADC Output Code Stitching

FIG. 6B shows an example block 656 for ADC output stitching, according to some embodiments. In some embodiments, the block 656 is a short first-in-first-out (FIFO) (e.g., a FIFO with a programmable depth of 10 samples). When a fast track event occurs, the few samples delay around the loop and the eventual time constant of the fast track compensation integrator 602 results in an inherent delay in reacting to the fast input signal. During those few samples, depending on the slew-rate of the analog CFBINA and PGA, the output may drift away towards saturation and the result will show as a glitch at the ADC output. Once those few delay samples elapse and the DAC output catches up with the input, the glitch is removed. In order to prevent this glitch from being passed, an optional ADC de-glitching or stitching (e.g., an algorithm performed by the block 656) is used, where the ADC output is delayed by a few samples (e.g., 10 samples) before being passed out. In some embodiments, in case of need for de-glitching a sample and hold in digital domain, the block 656 continues to stitch the output with the sample before the glitch occurred. In some embodiments, the block 656 is controlled by the adaptive baseline tracking algorithm 614.

FIG. 6E shows graph plots for the operation of example block 656 of output stitching, according to some embodiments. The example corresponds to stitching in an event of 10 mV input offset jump 658 (step-like input). Until the DAC catches up with the offset jump and in combination with a few samples of delay around the loop, the output glitches 660 (AFE output code raw shows the non-stitched output). The stitching flag from the state machine enables the sample and hold and the AFE output ADC code stitched 662 does not pass the glitch out.

| | | |
|---|---|---|
| Analog Front End High Frequency | 2000 Hz | High frequency baseline tracking |
| | | filter - fast track bandwidth |
| Analog Front End Low Frequency | 50 Hz | Low frequency baseline tracking |
| | | filter - slow track bandwidth |
| Analog Front End Ultra-Low | 20 Hz | Ultra-low frequency baseline tracking |
| Frequency | | filter for when baseline is quiet for an |
| | | extended period of time - ultra slow |
| | | track bandwidth |

### Example Parameters and Components for the Motion Artifact Algorithm

### Fast, Slow and Ultra-Slow Tracking Bandwidths

| | | |
|---|---|---|
| Analog Front End High Frequency | 2000 Hz | High frequency baseline tracking |
| | | filter - fast track bandwidth |
| Analog Front End Low Frequency | 50 Hz | Low frequency baseline tracking |
| | | filter - slow track bandwidth |
| Analog Front End Ultra-Low | 20 Hz | Ultra-low frequency baseline tracking |
| Frequency | | filter for when baseline is quiet for an |
| | | extended period of time - ultra slow |
| | | track bandwidth |

### Rate of Change Tracking Parameters Such as Monitor Timers, Gain Thresholds

| | | |
|---|---|---|
| Analog Front End Fast Track | 250e−3 | The fast track duration time after removal of |
| | | the condition - This time needs to elapse after |
| | | the rate of change drops below the lower |
| | | threshold before the fast track flag is removed |
| | | in order to switch from fast track to slow track |
| | | mode |
| Analog Front End Slow Track | 150e−3 | The slow track duration time after deassertion |
| | | of the flag - This time needs to elapse after the |
| | | fast track flag is removed before switching |
| | | from slow track to ultra slow track mode |

### Rate Monitor Threshold Settings

| | | |
|---|---|---|
| Analog Front End Fasttrack | 1.2*1000 | Amplitude threshold to be programmed above |
| Threshold | | the adaptive threshold for the rate monitor to |
| | | trigger fast track |
| Analog Front End Fasttrack | 1*500 | Hysteresis level below the th_fastrack for the |
| Hysteresis | | rate monitor to remove fast track mode |
| Analog Front End Adaptive | 25e−3 | Running average window length for |
| Threshold | | calculation of adaptive thresholding |

Gain applied to rate of change calculation—The choice of gain goes hand-in-hand with the threshold value settings

| | | |
|---|---|---|
| Analog Front End Gain Rate | 1 | ADC output de-glitching |
| | | stitch delay (in number of |
| | | samples); |
| | | AFE_spec.d_stitch_delay = |
| | | 10. |

Example details of the motion artifact algorithm are shown below, according to some embodiments.

| | Functions within | Programmability | Comments |
|---|---|---|---|
| Multi | A plurality of parallel | The FPGA includes | Low-pass corners: |
| Bandwidth | running channels (e.g., | backup filters at a | High speed tracking~(e.g., |
| Filters | 3, 4 channels) made of | substantially higher or | 500 Hz to 1 kHz) |
| | second order low-pass | lower bandwidth. | Low speed tracking |
| | Butterworth filters. | | (e.g., approximately 5 |
| | High speed tracking | | to 20 Hz) |
| | with low-speed | | Ultra-low speed |
| | tracking and ultra-low | | tracking (e.g., <1 Hz) |
| | speed tracking. | | |
| Rate of change | The functions include | Gain value is made | Programmable gain can |
| estimator | differentiators, | programmable. | be made POW2 for |
| | subtractors, a gain | Account for | multiplier |
| | function, and absolute | programmable delay | implementation. |
| | value calculation. | for differentiation delay | |
| Adaptive | An averaging filter is | Running average filter | An impulse response |
| thresholding | running and includes | impulse response | length range with an |
| | adding and subtracting. | length. | accuracy of a few |
| | | | hundred milliseconds |
| Baseline | The state machine | Up and down and | Timers keep time in the |
| tracking state | implements a hysteresis | hysteresis thresholds | order of a few hundred |
| machine | comparison and timers | can be programmable. | milli-second. |
| | for time keeping event | Timer time keeping | |
| | expiry and state | values can be | |
| | selection. | programmable. | |
| Integration | Some error signal | | Basic integration, gain, |
| function, add, | integrator function | | add, subtract are |
| subtract, gain | capabilities with bit | | included. |
| | depths (e.g., <32 bits) | | |
| | and running at same | | |
| | sample rate as the algo. | | |

### Example PLI Algorithm

In some embodiments, the PLI algorithm (blocks 124, FIG. 1) includes functions, such as adaptive notch filter, to estimate the fundamental frequency of PLI. In some embodiments, the algorithm includes discrete time oscillators to produce a limited number of harmonics (e.g., 7 PLI harmonics) of the detected fundamental. In some embodiments, the algorithm includes amplitude and phase estimation and adaptation through a recursive least squares (RLS) algorithm. In some embodiments, the compensation of PLI is in a feedback scheme. For example, the generated PLI signal by the algorithm is not subtracted from the system's output but it is applied to its input where it gets directly subtracted from the input signal. In some embodiments, the delay of the AFE and ADC are also within the RLS algorithm's loop and vice versa, the delay of some of the narrow band filters in the algo are in the feedback path, which can result in instability.

In some embodiments, RLS algorithm's error signal is the AFE's ADC output. The algorithm drives the DAC with the correct PLI signal such that the PLI at the ADC output converges to zero. In some embodiments, input filter phase delay is minimized. For example, the input Infinite Impulse Response (IIR) filter of 40-70 Hz used by the algorithm introduces significant phase shift and hence instability. The bandwidth may be set to the 6th harmonic of PLI (400 Hz). The settling parameters defined by the RLS algorithm is relaxed for slower convergence to ensure closed loop stability with the AFE and ADC delays in the loop. In some embodiments, an attenuation factor (e.g., a programmable factor) is added before the RLS algorithm signal chain for loop stability. In some embodiments, the PLI algorithm stays locked to the residual PLI after compensation. A hybrid DAC can be used.

In a feed forward open loop compensation scheme, functions can include PLI estimation, assuming PLI harmonics up to the Mth harmonic are tracked. In some embodiments, tracking is verified up to the seventh harmonic. In some embodiments, the algorithm receives the contaminated signal at the output of ADC, applies a narrowband BP filter (40-70 Hz IIR) and uses a lattice notch filter 718 to estimate its frequency. In some embodiments, the algorithm locks the frequency, phase and amplitude of a bank of digital oscillators (e.g., one oscillator per harmonic). In some embodiments, the outputs of oscillators are combined and subtracted from the incoming signal contaminated with PLI. The error is then feedback to the RLS algorithm.

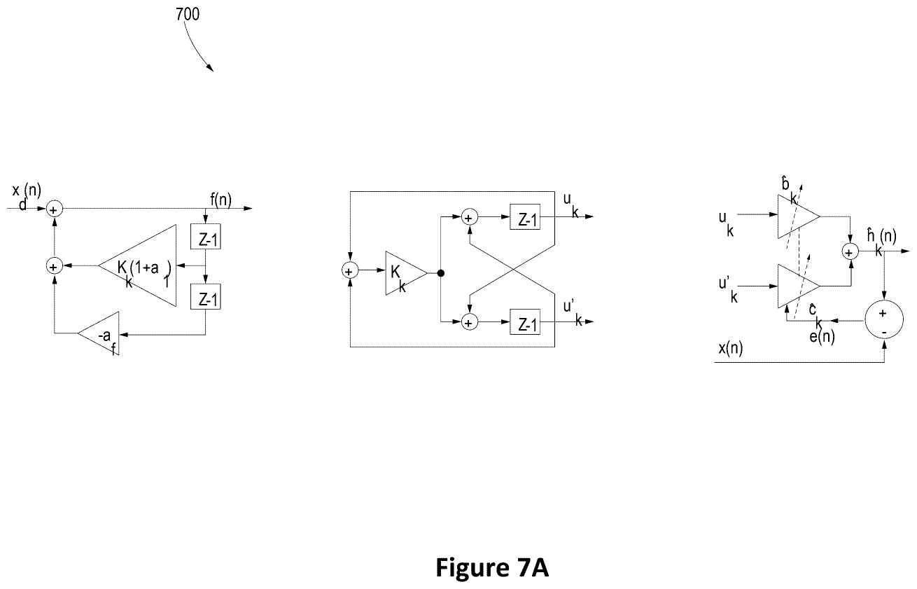

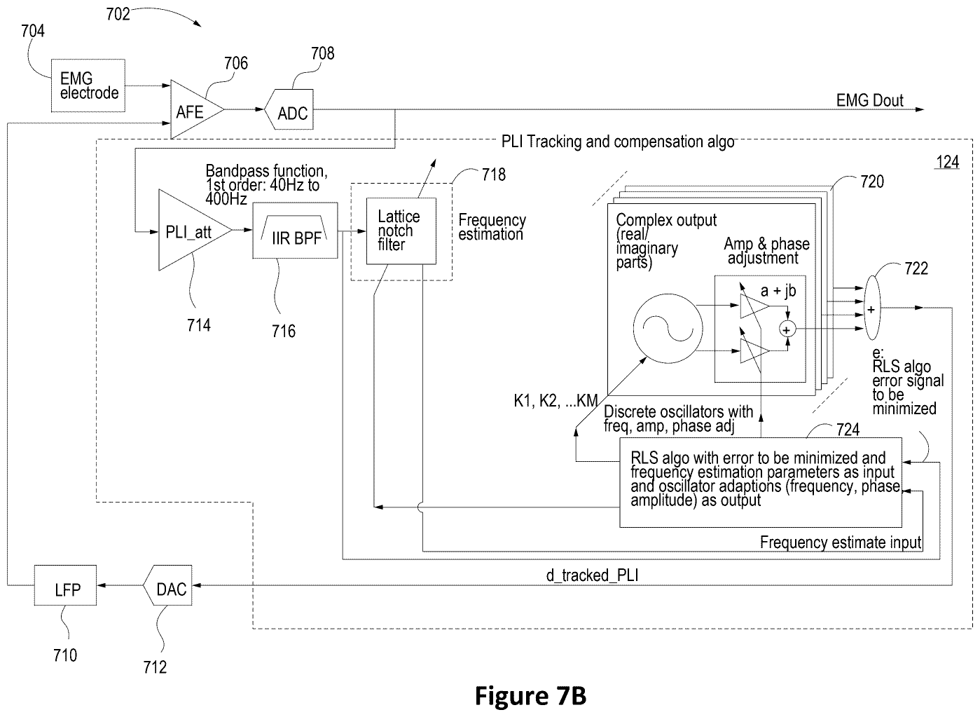

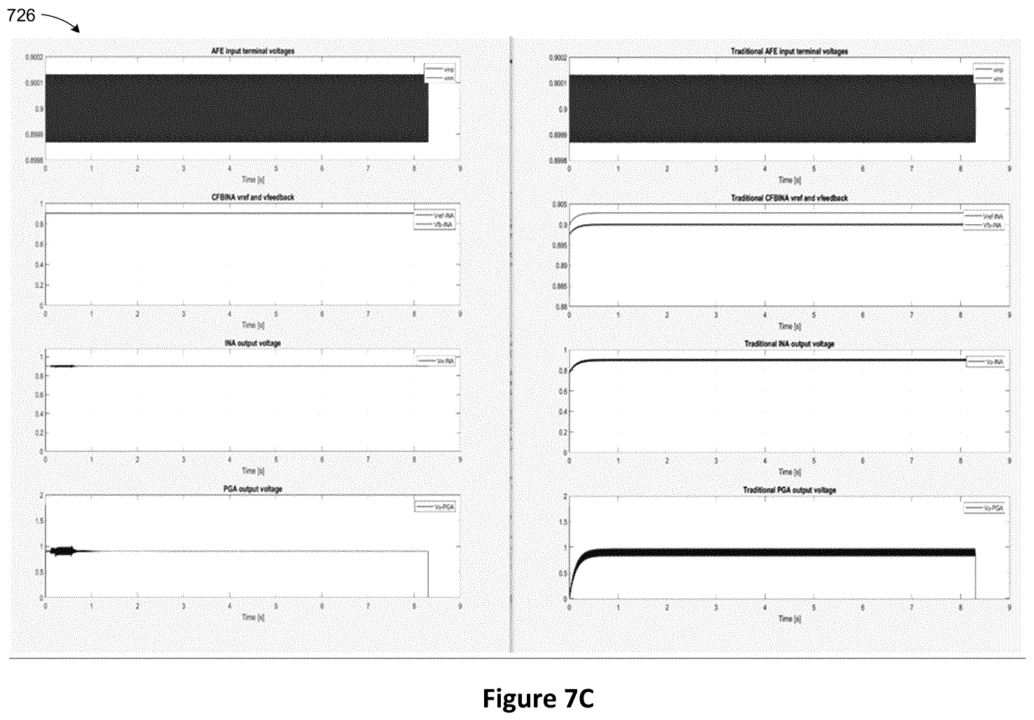

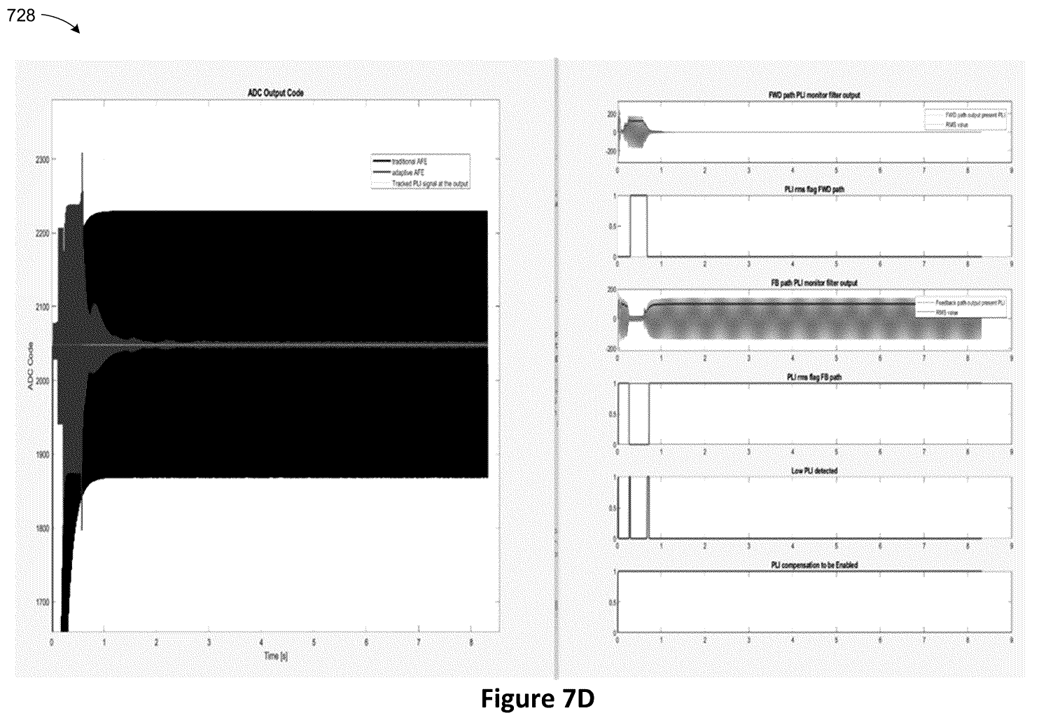

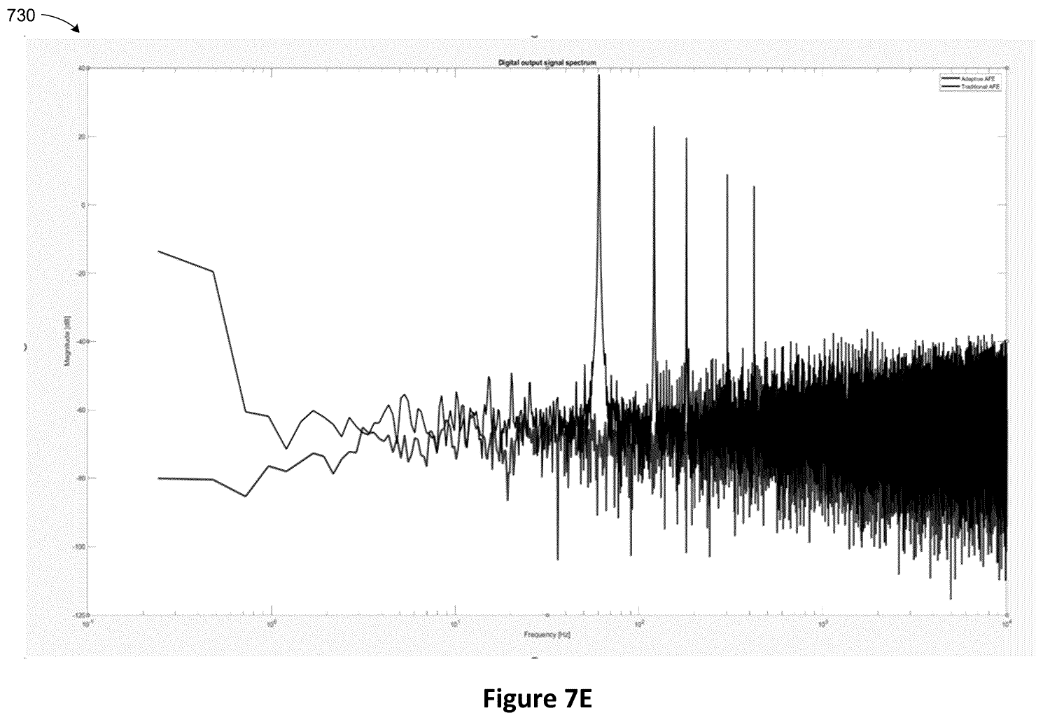

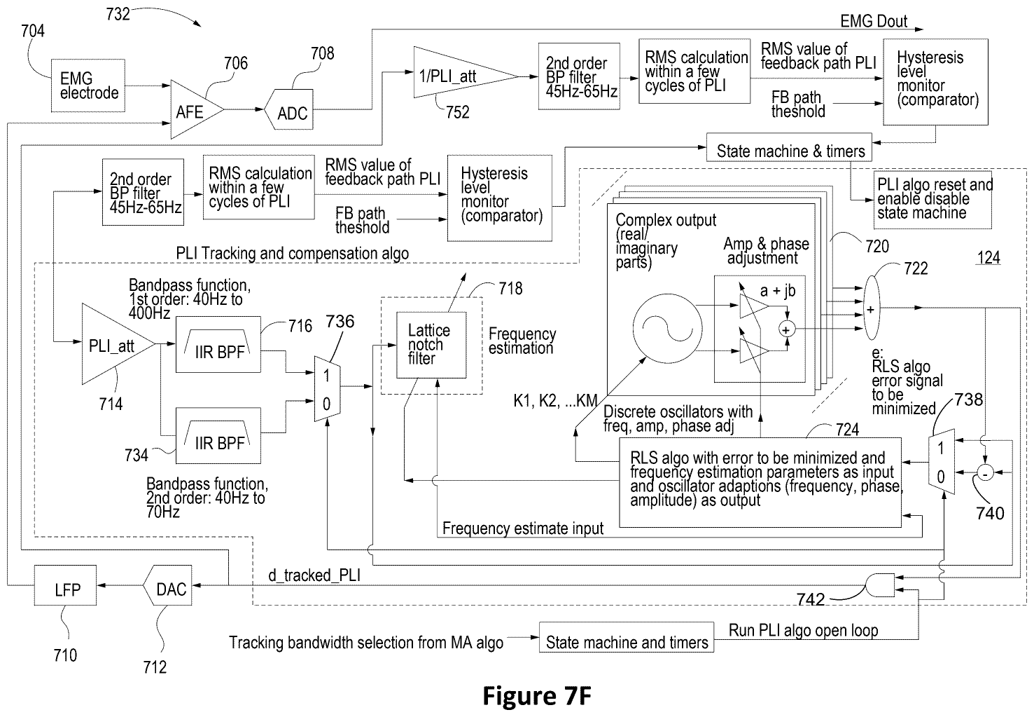

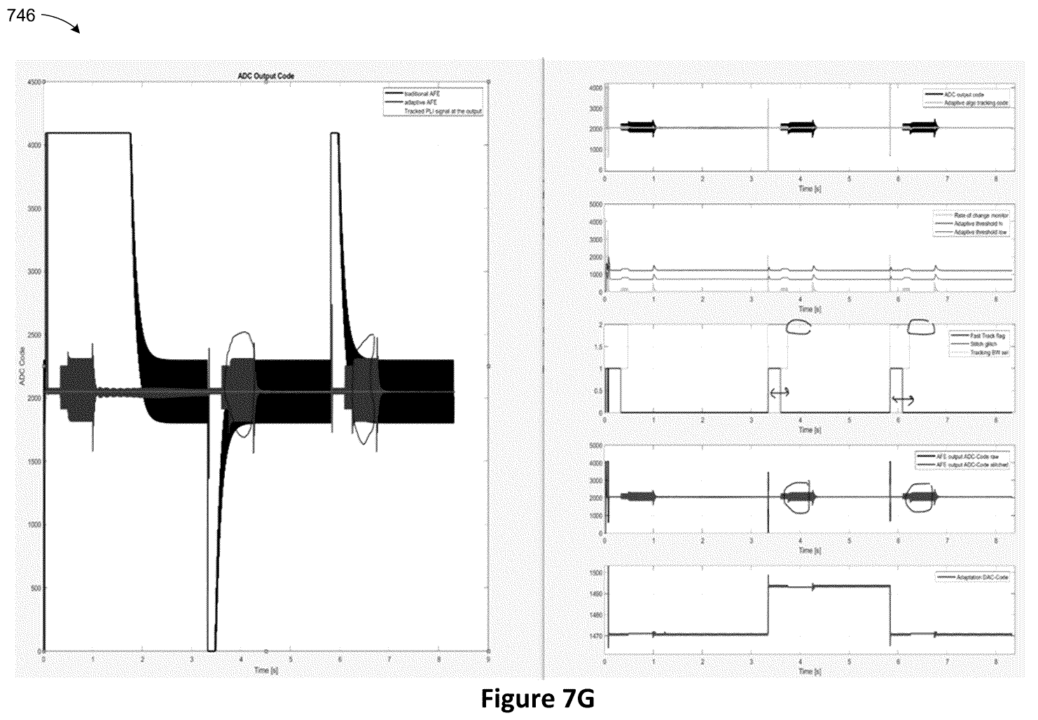

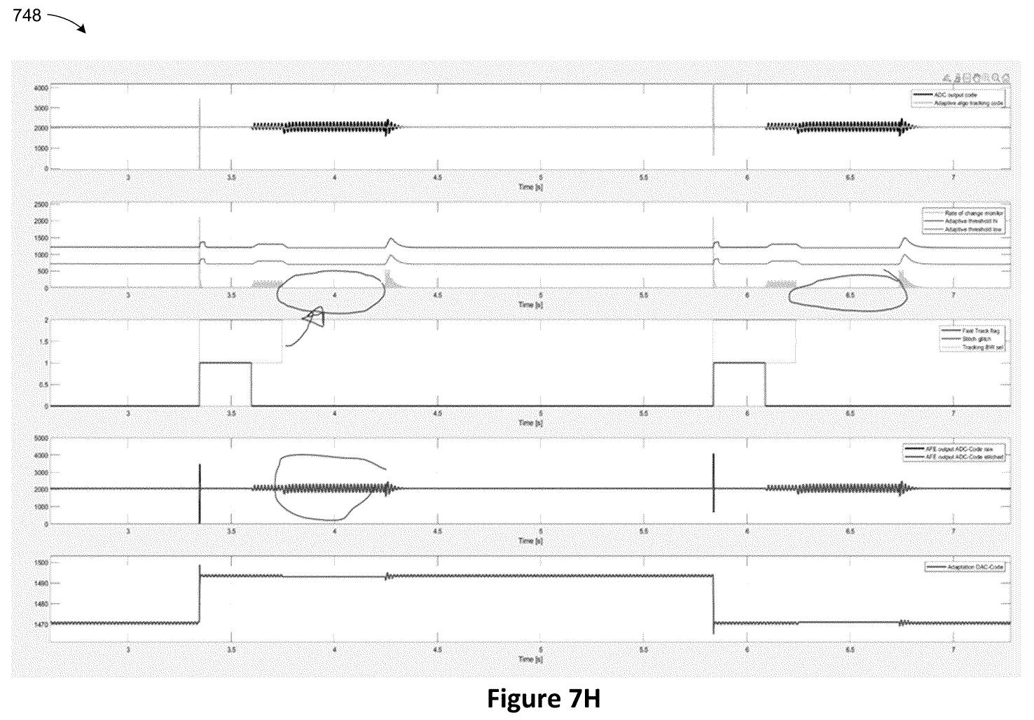

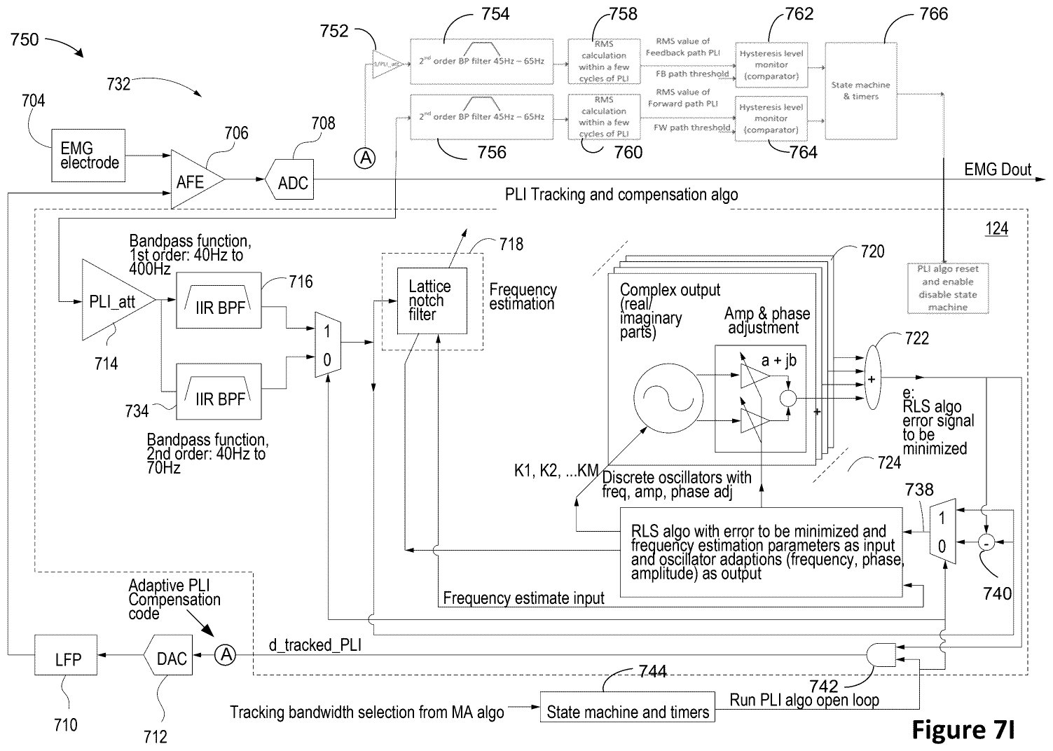

FIG. 7A shows block diagrams of example internal components 700 for PLI estimation algorithm, according to some embodiments. (a) shows lattice notch filter programmable through K and Alfa coefficients. (b) shows discrete-time oscillators. Kk determines frequency. Output feed to (c), which is an adaptive linear combiner to adapt phase and amplitude.

FIG. 7B is a block diagram of an example system 702 for implementing PLI tracking and compensation algorithm 124, according to some embodiments. Similar to FIG. 6A, an EMG electrode 704 is coupled, and provides EMG signals, to an analog frontend (AFE) 706, which is in turn coupled, and provides analog output, to an ADC 708. The AFE 706 is also coupled to, and receives signals from an LPF 710, which is coupled to, and receives inputs from a DAC 712. The DAC 712 receives adaptive PLI compensation code (d_tracked_PLI) from the PLI tracking and compensation algorithm 124. The PLI algorithm 124 drives the DAC 712 to present the estimated PLI (d_tracked_PLI) to the input of the AFE 706 at the CFBINA (described above).

In some embodiments, the PLI tracking and compensation algorithm 124 includes a PLI_att attenuation 714 receiving input from the ADC 708, and passing the attenuated signal to an Infinite Impulse Response Band-Pass Filter (IIR BPF) 716. The IIR BPF is a bandpass function, a first order filter, with bandwidth 40 Hz to 400 Hz, which helps reduce the phase shift introduced by this filter. The error signal (denoted by ‘e’) fed to an RLS algorithm 724 is present at the ADC output, i.e. as if the error signal post subtraction node at the analog input has been digitized by the ADC 708. The signal is captured after the IIR BPF 716. The delay due to that phase shift is within closed loop of the RLS algorithm 724. The adjustments of the algorithm 124 go through the AFE 706, the ADC 708 and the IIR BPF's delay before the algorithm 124 can see effect on the error signal ‘e’.

Some embodiments use a PLI_att attenuation factor 752 in the order of 1/64 to 1/256 to ensure stability. The settling speed parameters of the RLS is reduced (e.g., by approximately times 10) for the stability of the closed loop operation. The output of the IIR BPF 716 is fed to the lattice notch filters 718 whose notch frequencies are adjusted by the RLS algorithm 724 to estimate frequency (after notches are tuned, the output is driven to zero). The frequency parameters (K1, K2 . . . . KM) of the PLI harmonics 1, 2, . . . . M are then fed to M parallel running oscillators 720. Their complex outputs are then adjusted by two other parameters driven by the RLS 724 to have simultaneous adjustment of the phase and magnitude. The oscillator outputs (each oscillator produces one of the separate harmonics of the PLI) are combined (e.g., summation 722) to generate the actual estimation of PLI, d_tracked_PLI, which then drives the DAC 712 to generate an equivalent analog version of the PLI. The DAC signal is adjusted until the error ‘e’ at the ADC output (tapped into the output of the IIR BPF) is driven to zero.

In some embodiments, the overall PLI tracking loop runs at the same sample rate as the ADC (e.g., 20 kHz). Some embodiments use higher frequencies closer to the back end of the algorithm's signal path, which helps with convergence of higher order harmonics. In some embodiments, the d_tracked_PLI code is combined with the output of the motion artifact compensation loop, examples of which are described above in reference to FIGS. 6A-6E.

FIGS. 7C, 7D and 7E show graph plots for example operations of the PLI algorithm, according to some embodiments. The graph plots 726 (FIG. 7C) compare the adaptive AFE analog signals (left) with a traditional AFE (right) for a relatively moderate amount of PLI (e.g., 500 uVpk). Graph plots 728 (FIG. 7D) compare ADC outputs (left) with internal PLI level monitoring signals (right). Graph plots 730 (FIG. 7E) compare the spectra of ADC output for adaptive AFE analog signals (red) and traditional AFE with no compensation (black).

### Closed Loop Operation Challenges-Residual PLI's Signal-to-Noise Ratio (SNR)

The architecture of closed loop compensation results in the PLI algorithm monitoring the residual PLI post compensation. This is simply due to the fact that if the algorithm performs well in replicating the PLI to an accurate level in terms of harmonics, magnitude, phase and frequency, the algorithm will cancel most of the PLI at the AFE input, which means the ADC output should ideally contain zero PLI. Naturally, this cannot happen due to the limited loop gain and accuracies of the calculations and the DAC's limitations in accurately generating the estimated input referred PLI signal. Therefore, it is beneficial to feed the RLS algorithm's error signal input (‘e’ input) with the output of the IIR BPF to help with the SNR at the PLI frequency. However, due to the other challenge of closed loop compensation being the excess delay within the RLS adjustment and error monitoring in the feedback system, the IIR BPF is made less selective by increasing its bandwidth.

### Compensation Signal SNR

The input referred compensation signal needs to go through digital-to-analog conversion, where the quantization error of the DAC will impact the capability of RLS locking specially the higher harmonic oscillators (smaller amplitude, more prone to phase error). With a 12 bit DAC, the harmonics beyond the second harmonic cannot be locked and the oscillators will go insatiable in terms of frequency lock. A 16 bit DAC should be sufficient and hence the hybrid DAC architecture described above can be used.

### Interactions Between the Motion Artifact Algorithm and PLI Algorithm

The motion artifact (MA) algorithm reacts to large motion artifacts by adapting the servo path's bandwidth, so the PLI algorithm might be thrown into instability. One way to observe that is that when the MA algorithm opens up bandwidth, it filters all of the PLI as the bandwidth of the feedback path is increased to much higher frequencies than PLI and so the PLI signal more or less disappears from the forward path. So the PLI algorithm loses lock. Accordingly, some embodiments use one or more handshakes between the algorithms such that when the MA algorithm enters high speed tracking, the PLI algorithm is disabled, i.e. the PLI tracking DAC code is ignored and not fed to the DAC and the IIR BPF's memory is reset. After the MA algorithm settles, the PLI algorithm needs to come back and lock to the PLI, however that can take time and sometimes the bring up and locking of the PLI algorithm can cause fast transitioning signals that end up triggering the MA algorithm. This might end up in a continuous trigger of that algorithm. In some embodiments, the MA algorithm is disabled when the PLI algorithm is settling.

FIG. 7F is a block diagram of another example system 732 for implementing the PLI algorithm 124, according to some embodiments. To bring up the PLI quickly after MA event passes, some embodiments use a process where the PLI algorithm is first locked in an open loop manner, e.g., the DAC code is frozen and the RLS algorithm error signal ‘e’ is generated by subtracting the IIR BPF output from the combined oscillator outputs. This is shown in FIG. 7F with the red signal path. Furthermore, the IIR BPF is also switched to a narrow band second order IIR BPF path 734 to multiplexer 736, to provide more selectivity and case the settling. The result is a quick settling of frequency and phase but the amplitude will be off as it is output referred. A timer 744 keeps open loop operation time. After the timer expires, the RLS error signal input is turned back (components 738 and 740) to the wide band first order IIR BPF 716 and the DAC code is released 742 to drive the compensation.

In some embodiments, this provision helps make the recovery of the PLI loop more robust in presence of large amplitude motion artifacts. This step helps have the PLI tracking in feed forward (FFWD) during initialization to let RLS algorithm parameters mainly on frequency to settle before closing the loop. When mask_pli_code is 0 (when coming out of a fast-track MA compensation transition) or when flag_state_enable_trackpli is 0 (when no above threshold level of PLI rms levels detected) then the feedback code for PLI compensation is masked (no closed loop cancellation) and the RLS algorithm's residual error shifts to forward path (rather input of the feedback system) by subtracting the narrowband BPIIR from the oscillator output.

FIG. 7G shows graph plots for example simulation 746 of the algorithm described above in reference to FIG. 7F when recovering from a large motion artifact, according to some embodiments. A large offset step is applied in middle of the simulation (see right bottom row where DAC code is shown jumping to an offset level to compensate for that. During the highlighted areas with blue circles, the PLI algorithm is driven open loop to settle. Then it is turned into closed loop. During the times marked on the right side with blue arrows, the PLI algorithm is disabled while large motion artifact tracking and settling is ongoing.

Another interaction between the PLI algorithm and MA algorithm is due to PLI algorithm's initial settling resulting in the MA algorithm's rate of change monitor being triggered. To prevent that, in some embodiments, an initial PLI compensation mask period disables the rate of change monitor signal for a period of time, right when the PLI is activated when coming out of a fast MA recovery (i.e. right after the bw_sel signal selects the ultra low bandwidth mode). This timer is programmable. FIG. 7H shows graph plots for example operation 748 for disabling the rate of change monitor signal, according to some embodiments. This period when MA rate of change is disabled is marked in FIG. 7H with red circles.

### Example PLI Monitoring Functions

In some embodiments, the PLI compensation loop monitors the amount of PLI in forward and feedback paths, calculates the root mean square level of PLI in both paths and compares that with programmable thresholds to determine the PLI level on both paths. If there is a large PLI in the forward (FW) path and small PLI on the feedback (FB) path, this means that the loop is not locked or compensating. If there is small PLI in the FW path and large PLI in the FB path, this means that the loop is locked and is compensating the PLI at the input. If there is small PLI in both FW and FB, the PLI is too small, so some embodiments disable the loop.

FIG. 7I is a block diagram of another example system 750 for implementing the PLI algorithm 124, according to some embodiments. The block diagram shows additional components of the PLI monitoring. The FFWD and FB paths have their own second order narrowband bandpass filters (filters 754 and 756, respectively) followed by programmable length RMS calculators (calculators 758 and 760, respectively) that determine the amount of energy in the feedback and feedforward paths. The root mean square values are monitored by hysteresis level comparators 762 and 764 with programmable thresholds and their outputs determine high or low PLI levels in the feedback and feedforward path, respectively. These are fed to a state machine 766 that decides whether PLI compensation should be disabled or not based on the present PLI level in the signal paths. The state machine can include detection of PLI loop lack of lock or instability, according to some embodiments.

### Example Parameters and Components for PLI Algorithm

Example parameters for the PLI tracking and compensation algorithm are shown below. One example combination includes working with a 14-bit DAC.

| | | |
|---|---|---|
| Analog Front End PLI M | 3 | |
| Analog Front End PLI B | [100, 0.01, 0.1] | |
| Analog Front End PLI P | [0.1, 2, 0.1] | |
| Analog Front End PLI W | 0.1 | |
| Analog Front End PLI | 1/1024 | |
| Attenuation | | |
| Analog Front End Initial PLI | 500e−3 or 250e−3 | The duration of time when the |
| Comp Mask | | MA rate of change is disabled |
| | | to let PLI settle and not re- |
| | | trigger it. |
| Scaling factor | 1 | A scaling factor. 1 is one |
| | | example value but not |
| | | limiting. |
| Analog Front End PLI M | 7 | M is the number of harmonics |
| | | to remove. |
| Analog Front End PLI B | [100, 0.01, sc*0.25] | Notch filter characteristics |

### Notch Filter Characteristics

| | | |
|---|---|---|
| Analog Front End PLI B | [100, 0.01, sc*0.25] | Notch filter characteristics |
| B contains three elements | [B0, Binf, Bst] | B0, Initial notch bandwidth of |
| | | the frequency estimator; Binf, |
| | | Asymptotic notch bandwidth |
| | | of the frequency estimator; |
| | | Bst, Rate of convergence to |
| | | 95% of the asymptotic |
| | | bandwidth Binf |
| Analog Front End PLI P | [0.1, 1, sc*0.25] | Frequency estimator |
| | | characteristics |
| P contains three elements | [P0, Pinf, Pst] | P0, Initial settling time of the |
| | | frequency estimator; Pinf, |
| | | Asymptotic settling time of |
| | | the frequency estimator; Pst, |
| | | Rate of convergence to 95% |
| | | of the asymptotic settling |
| | | time |
| Analog Front End PLI W | sc*0.25 | Settling time of the amplitude |
| | | and phase estimator |
| Analog Front End PLI | 1/128 | Closed loop compensation |
| Attenuation | | loop gain attenuation factor. |
| | | Reduce loop gain, e.g. 1/1024 |
| | | when tracking higher |
| | | harmonics say up to 7^{th} |
| Analog Front End Forward | 100 | Forward path narrow band |
| Path | | PLI energy monitor threshold |
| | | for detection of PLI in |
| | | forward path |
| Analog Front End Hysteresis | 50 | FWD path hysteresis level |
| Level Below Threshold | | below threshold to de-assert |
| | | flag |
| Analog Front End Narrow | 80 | Feedback path narrow band |
| Feedback Path | | PLI energy monitor threshod |
| | | for detection of PLI in FB |
| | | path |
| Analog Front End Feedback | 50 | Feedback path hysteresis |
| Path Hysteresis Level Below | | level below threshold to de- |
| Threshold | | assert flag |
| Analog Front End Forward | 50e−3 | This is the amount of time a |
| PLI Timer | | higher than threshold PLI rms |
| | | level should be detected |
| | | before the loop decides there |
| | | is PLI in the forward path |
| Analog Front End without PLI | 250e−3 | A timer that is triggered when |
| Timer | | no PLI detection mode is |
| | | detected and after this time |
| | | elapses, the PLI loop is |
| | | disabled. Once PLI is |
| | | detected we come out of this |
| | | mode. |

Example details for the PLI algorithm are shown below, according to some embodiments.

| | Functions within | Programmability | Comments on spec |
|---|---|---|---|
| Bandpass filtering | Bandpass IIR filters | Passband | Passband of 40 Hz to |
| | with third or fourth | programmability | 70 Hz or maybe |
| | order filtering | (potentially needed) | narrower band |
| | Differentiator | | optimized for 60 Hz |
| | functions | | only or 50 Hz only |
| Frequency | Adaptive notch filters | Lattice all-pole | Needs delays and some |
| estimation | used for frequency | adaptive notch filter | multiplication and |
| | estimation (rather than | coefficients | addition. |
| | filtering) | | |
| Harmonic | Discrete time | Oscillator parameters | Needs delays and some |
| generation/discrete | oscillators | (1 or 2 coefficients) | multiplication and |
| oscillators | | | addition. |
| Amplitude/Phase | RLS algorithm to | Optional | 32-bit bit depth for |
| estimation | adapt the harmonics | | registers, 10-15 |
| | | | registers, some weight |
| | | | multiplication/addition |
| Some integration | Some error signal | | Some basic integration, |
| function, add, | integrator function | | gain, add, subtract will |
| subtract, gain | capabilities with bit | | eventually be needed |
| | depths <32 bits and | | |
| | running at same | | |
| | sample rate as the | | |
| | algorithm | | |

In some embodiments, the PLI algorithm targets 50 Hz and/or 60 Hz powerline noise sources. In some embodiments, the PLI algorithm tackles other narrowband noise sources with known frequency ranges. An example application is the multi-sensor coexistence use-case where cross-modality interference issues can be mitigated in multi-sensor device. For example, suppose a wristband has a 100 Hz touchscreen attached to it, which can introduce narrowband noise to EMG measurements at that frequency. The PLI algorithm described herein can be used for mitigating such noise by setting the bandpass frequency at the neighborhood of 100 Hz, for example, instead of setting it at 40-70 Hz. As such, in multi-sensor devices, EMG signal can be protected from noise emitted from other sensor modalities.

### Example Template Detection

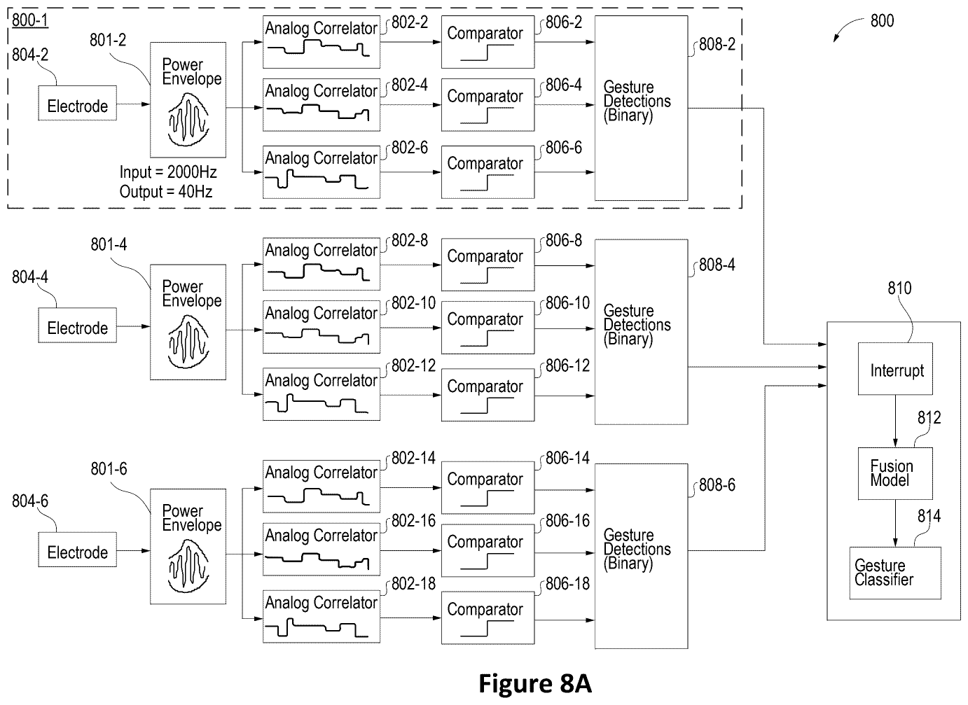

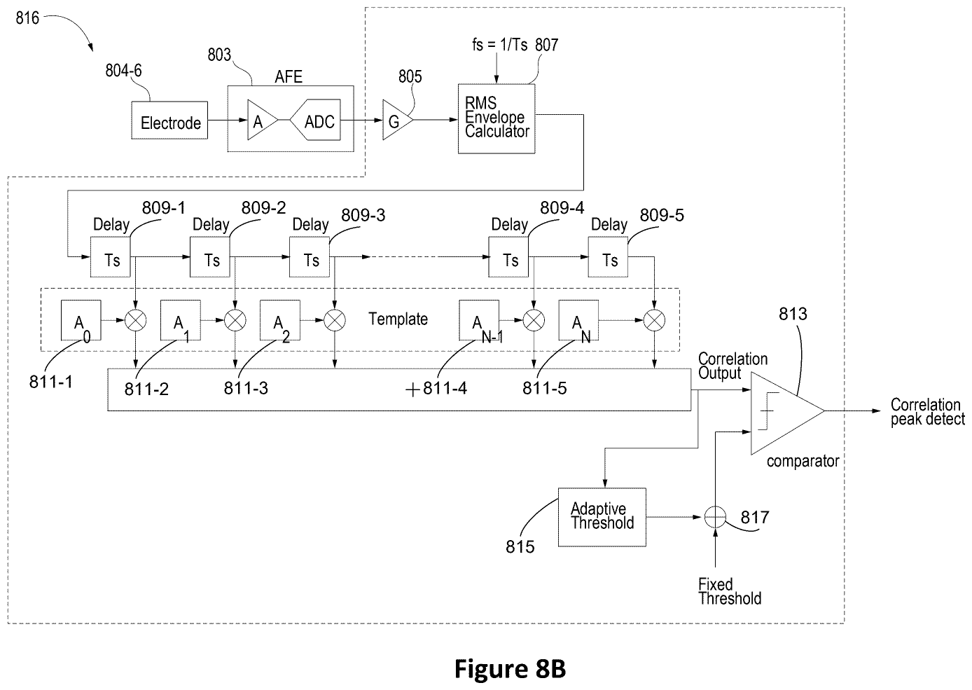

FIG. 8A-8C illustrate an example of providing EMG-based gesture recognition that can achieve orders of magnitude better energy efficiency compared to traditional methods.

FIG. 8A is a block diagram of an example system 800 for EMG-based gesture recognition, according to some embodiments. Comparing the analog signals for specific gestures and then compares it. Identifies the gesture based on the closest match. Here it's doing this in parallel per electrode. It does each in parallel. The one that is the closest is the one it selects.

FIG. 8A further illustrates a system 800 generating single-channel gesture detections, without the use of any analog-to-digital converters, and instead using analog correlators (e.g., analog correlator's 802-2 to 802-18) on EMG power features. In some embodiments, single-channel gesture detections are sparse events, which create interrupts for a central processor, which then wakes up and fuses the sparse events into a single gesture classification results. Some embodiments (1) perform the dense data processing at the analog layer, (2) eliminate the use of ADCs, and/or (3) reduce the data rate to the central processing node (e.g., detections are sparse, while EMG data is dense). This can result in an extremely power-efficient gesture recognition system that can then be used for wake-up gestures and/or an ultra-low-power mode for EMG devices.

Some embodiments emulate hardware components as part of a trainable machine learning model, and then train that model for gesture targets (e.g., surface taps and swipes, such as left, right, up, down). The resulting model is then deployed to a device. In some embodiments, gesture signals are trained by a neural network to determine the correlation between signals and gestures. Signals can be processed as voltage or power versus time data which then serves as the input to the machine learning model. Training of this machine learning model can be performed in the software until a satisfying result is achieved to detect desired gestures accurately. Then the parameters of the machine learning model can be deployed into the hardware as a hard-coded hardware model to detect the gestures. In some embodiments, hardware components, such as the analog correlators, are emulated in software simulations with programmable parameters. These parameters are then trained using supervised machine learning approaches, such as neural networks, linear classifiers, or discriminant analysis, against labeled EMG data and then the learned parameters are optimally quantized to the desired bitwise depth, such as 2-bits or 4-bits for analog correlators, for hardware instantiation. In some embodiments, after the overall architecture is validated, hardware constraints are evaluated for gesture recognition.

For template length, some embodiments include up to a predetermined number (e.g., 10) of analog correlator weights per channel per gesture, for a predetermined set of target gestures (e.g., 3-5 gestures). For quantization, the analog correlator weights are quantized with a target quantization of 2-bits. Some embodiments use up to 4-bit quantization, but there is a trade-off between extra bits and the number of correlator weights. Some embodiments pre-train the analog correlators from a limited number of examples (e.g., 20 samples) from any given user. For example, data for the target gestures of thumb clicks and swipes (e.g., left, right, up, down) may be used. Using this methodology, under the constraint of 2-bit quantization, and with template lengths of 20 samples, experiments showed 86% average precision in some instances.

In some embodiments, analog correlators (e.g., analog correlator's 802-2 to 802-18) are trained for gesture recognition using a supervised learning approach, where labeled examples of different gestures are provided to the system during a training phase. Some embodiments collect a dataset of sensor data corresponding to different gesture patterns to be recognized. This data is labeled with the specific gesture it represents. The collected sensor data may need pre-processing steps, such as filtering, normalization, or segmentation, to prepare it for the training process. Relevant features are extracted from the pre-processed sensor data that can effectively capture the characteristics of different gestures. Techniques like Fourier analysis, wavelet transforms, or statistical measures can be used. For each gesture class, a template or reference pattern is generated based on the extracted features of the labeled examples in the training data. This template represents the expected pattern for that gesture. Some embodiments configure the analog correlator hardware to perform template matching. This involves mapping the extracted features and generated templates to the configurable parameters of the analog correlator, such as delay line taps, weight values, or correlation kernels. During the training phase, the analog correlator is exposed to the labeled examples of different gestures from the training data. The configurable parameters of the correlator are adjusted iteratively to minimize the error between the output and the expected gesture labels, effectively learning to recognize the different gesture patterns. After training, the performance of the analog correlator can be evaluated on a separate validation or test dataset to assess its gesture recognition accuracy and make any necessary adjustments. Different training algorithms can be used depending on the analog correlator architecture, the sensor modalities used, and/or the complexity of the gestures being recognized. Additionally, techniques like transfer learning or semi-supervised learning can also be used if labeled data is limited.

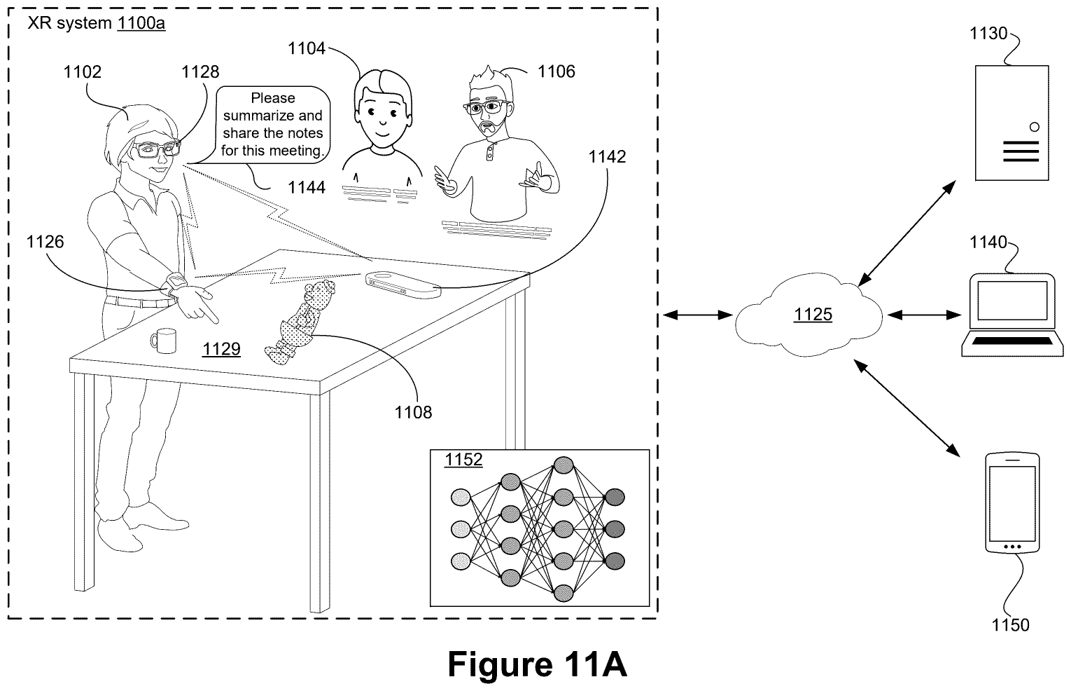

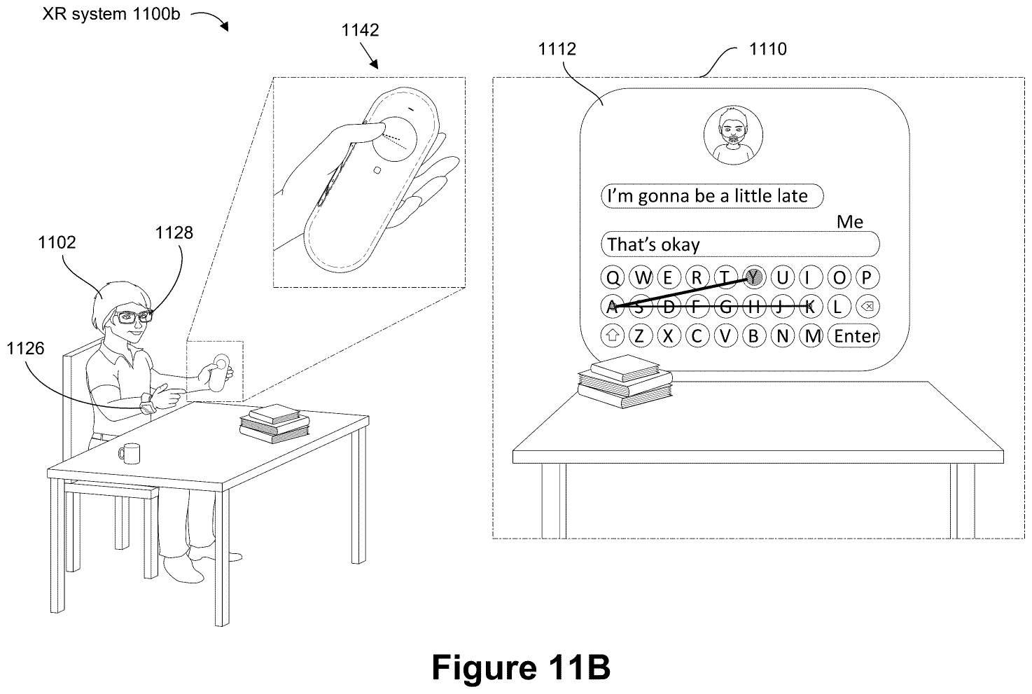

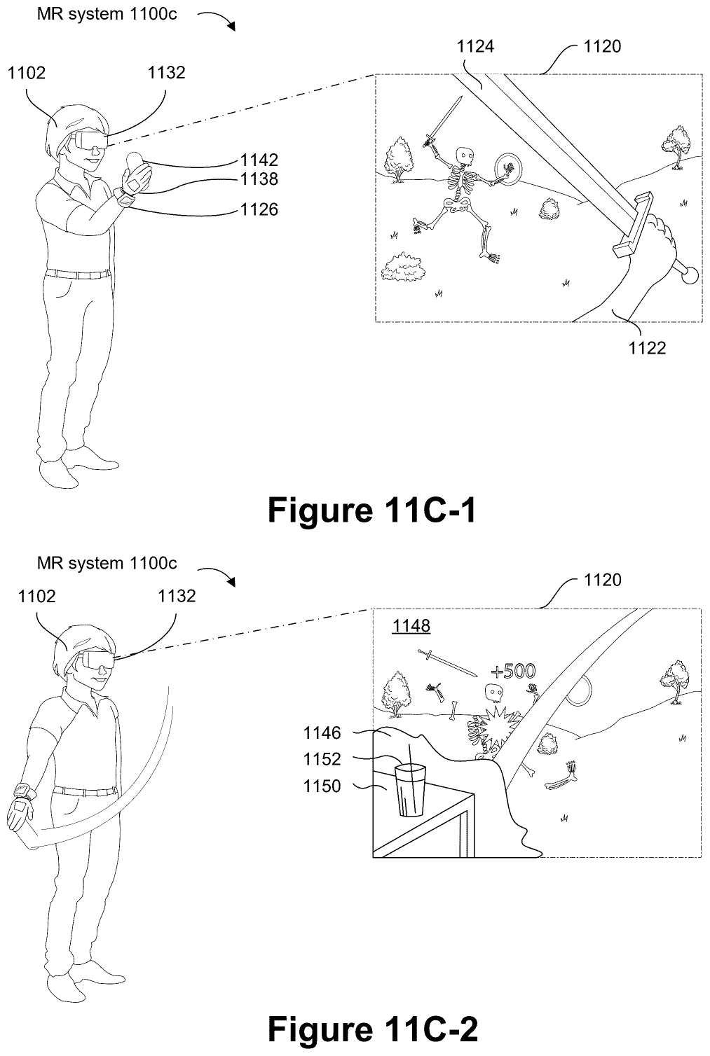

For example, FIG. 8A further illustrates one or more electrodes (e.g., electrodes 804-2-804-6) representing one or more electrodes coupled to (e.g., integrated into) the band portion of a wrist-wearable device (e.g., wrist-wearable device 1126; FIG. 11A). The system 800 in FIG. 8A is configured to measure a noisy signal (e.g., movement by a user, gesture by a user) detected via the one or more electrodes and process the signal such that the system can make a gesture classification. For example, the electrode receives a signal based on movement from a user performing a pinch gesture to select a UI element.

The band portion of the wrist-wearable device wrist-wearable device 1126; FIG. 11A can include a plurality of electrodes configured to detect movement by the user and generate a noisy signal. Each respective electrode can include their own respective system configured to detect one or more signals and process signals individually long their respective system. For example, electrode 804-2 is part of system 800-1 and the signal generated from electrode 804-2 is processed within system 800-1. Each system 800 can include multiple electrodes with their own respective system. The signal generated by the plurality of electrodes is sent to a power envelope 801-2.

In some embodiments, the system 800 includes a plurality of power envelopes (e.g., power envelopes 801-2 to 801-6) configured to receive a signal from a respective electrode and provide a banded signal to a plurality of analog correlators (e.g., analog correlators 802-2 to 802-18). Each respective power envelope receives a noise signal from the electrode and process the signal to remove additional nose and provide a cleaner signal to at least one or more analog correlators. For example, the respective power envelope can receive a 2000 Hz signal and output a 40 Hz signal.

Each respective system such as system 800-1 can include a plurality of analog correlators. For example, each electrode coupled to the band portion of the wrist-wearable device can include multiple analog correlators such as analog correlators 802-2 to 802-6 within system 800-1. Each analog correlator is associated with and trained for a respective gesture. Each respective analog correlator receives the output of the power envelope, performs a correlation calculation, and outputs the calculation to the respective comparator. For example, analog correlator 802-6 receives the output of power envelop 801-2 and outputs the calculation to comparator 806-6. When the gesture detected by the electrode matches the gesture associated with the respective analog correlator, the input to the comparator (the output of the analog correlator) is configured to trigger the comparator (e.g., render a high value). When the gesture detected by the electrode does not match the gesture associated with the respective analog correlator, the input to the comparator is configured to generate a low signal.

Each respective analog correlator includes a respective comparator. For example, analog correlator 802-8 outputs a calculation to comparator 806-8. Each respective comparator evaluates the calculation from the analog correlator and if the calculation is above a certain threshold, the comparator indicates that the respective gesture has been detected. If the calculation is below a threshold, the comparator does not indicate the gesture has been detected. For example, the comparator will output high if the gesture is detected.

A plurality of comparators (e.g., comparator's 806-14 to 806-18) outputs to a gesture detector 808-6 configured to receive a digital (e.g., binary) output of at least one or more comparators. The gesture detector is configured to determine which gestures (e.g., gestures associated with each respective analog correlator) have been performed. For example, an electrode could include several (e.g., 15) analog correlators configured to distinguish between several gestures.

At least one or more gesture detectors (e.g., gesture detectors 808-2 to 808-6) is configured to output a binary to an interrupter 810. The interrupter is configured to receive the binary outputs from the one or more gesture detectors and output to the fusion model that a gesture might be detected. For example, if one of the comparator's outputs high indicating a gesture is detected, the gesture detectors will pass that to the interrupter 810. Until a gesture is detected, the interrupter will stay inactive.

The fusion model 812 is configured to evaluate the inputs, receive the gesture information, and ultimately output the results to the gesture classifier 814. The gesture classifier 814 outputs the result to the system.

FIG. 8B is a block diagram of an example circuit 816 for a digital equivalent of an analog 1-D correlator (e.g., analog correlator's 802-2 to 802-18) coupled to an analog front-end (e.g., AFE 803), according to some embodiments. described above as well. Electrode 804-8 includes features analogous to electrodes 804-2 to 804-6. In some embodiments, the electrode detects one or more movements of a user and generates a signal. The AFE 803 receives the signal and converts it from an analog signal to a digital signal and outputs the signal to a signal conditioning amplifier 805. The signal conditioning amplifier 805 amplifies the output of the AFE 803 (e.g., now a digital signal). The amplified digital signal output from the signal conditioning amplifier 805 is received by the RMS envelope calculator 807. The RMS envelope calculator includes features analogous to the power envelopes (e.g., 801-2 to 801-6; FIG. 8A) and bands the digital signal to reduce the noise. In some embodiments, the frequency the signal is banded to is 1/Ts (e.g., Ts=the sampling period).

The output of the RMS envelope calculator 807 is a digital signal that is sampled by delays (e.g., delays 809-1 to 809-5), multiplied by coefficients (e.g., coefficients 811-1 to 811-5) and then summed to generate the correlation output. The process of sampling using the delays and multiplying by coefficients produces an analogous results to that discussed with respect to the analog correlators in FIG. 8A. The coefficients 811-1 to 811-5 are adjusted based on the respective gesture the correlator is showing. The correlation output is received by a comparator 813 which includes analogous features to the comparators discussed in FIG. 8A. The adaptive threshold 815 provides a way to adjust the signal as needed and is combined with the fixed threshold 817 and received by the input of the comparator. The output of the comparator 813 generates the correlation peak detect.

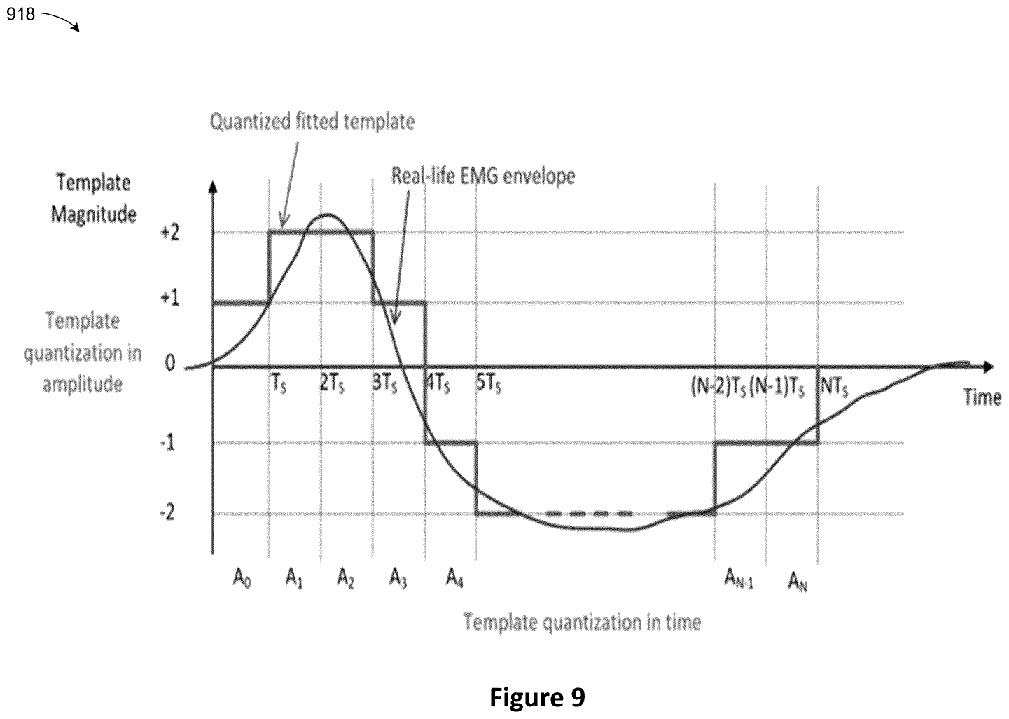

FIG. 9 shows an example envelope template 918 in accordance with some embodiments. FIG. 9 further illustrates the output of the ADC shown in FIG. 8B.

### Example Adaptive Successive Approximation Register (SAR) ADC Bypass

Some embodiments predicting ADC values use a SAR architecture. SARs are uniquely suited for prediction because they convert each bit independently, and skipping a bit saves energy by allowing the SAR to idle a larger percentage of the time. Additionally, early bits consume more energy than later bits, so skipping the early bits saves additional energy. In some embodiments, a math block generates a prediction of the input signal and a confidence level expressed in bits. The SAR fills the provided values into its SA register and skips the associated comparison cycles. When the prediction is correct, it achieves a power reduction based on the number of steps skipped and the size of the capacitors skipped. When the prediction is incorrect, it wastes at most two conversion cycles before it discovers the error, at which point it can do one of the following: (1) emit an error code that optionally includes a value (e.g., “Input is above 0.123V”); (2) full re-convert. At data rates for applications described herein, the SAR will be inherently faster than the sample rate of concern, so this is just an energy penalty; and (3) partial re-convert. Since the error case has some information about the value, it may be able to skip some conversion steps in its second attempt.

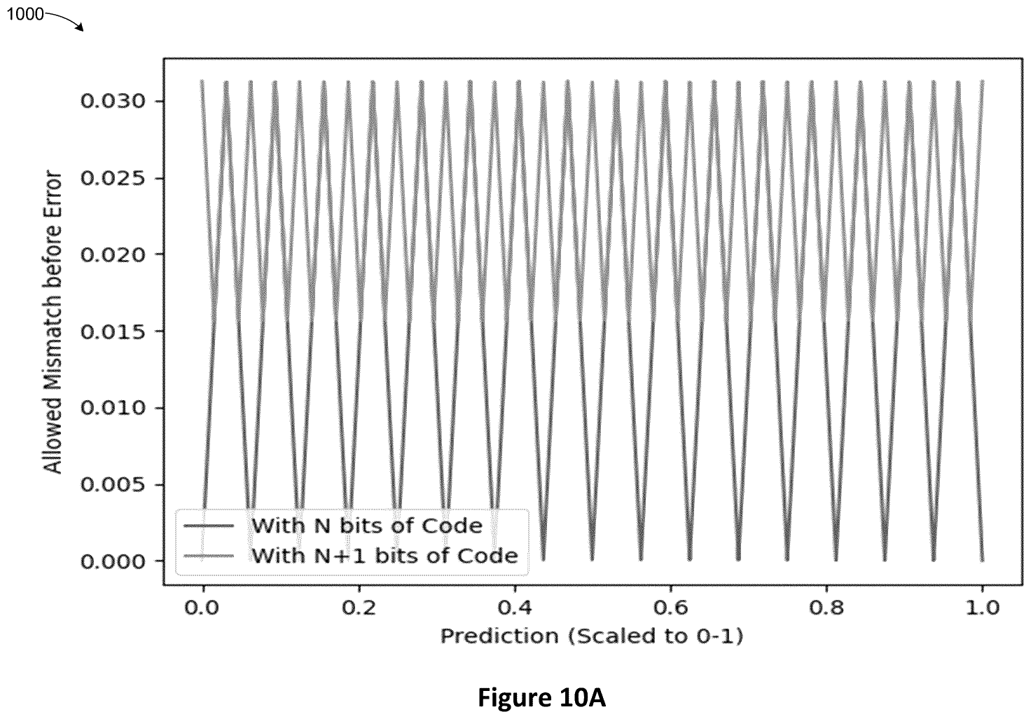

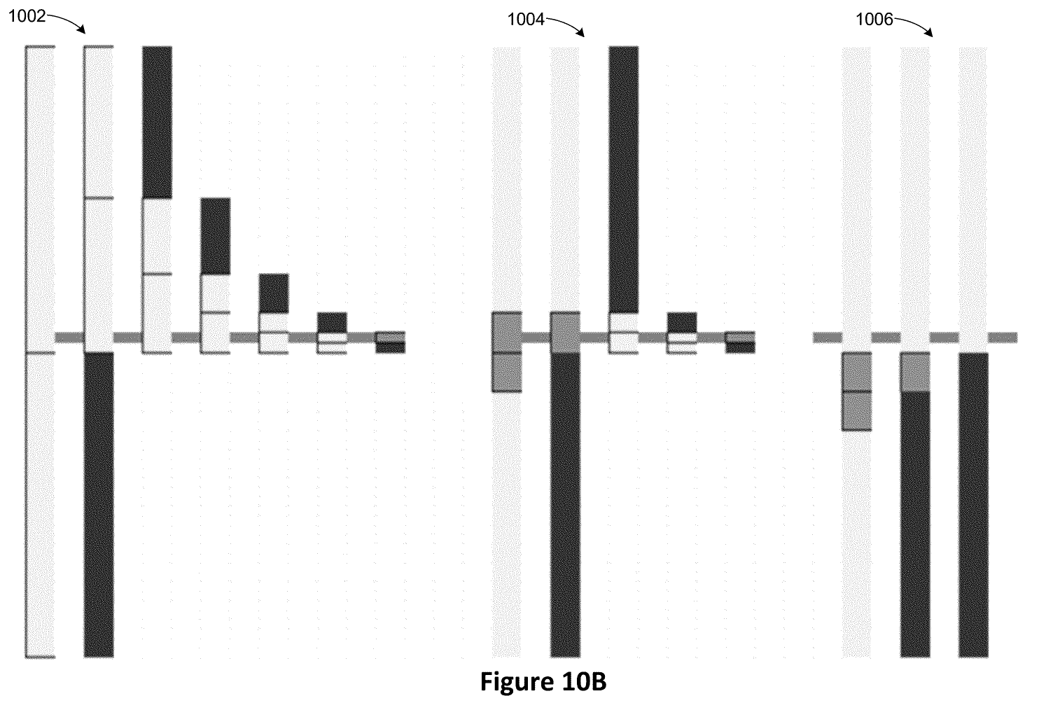

Prediction intervals are defined by their depth (N) and value (C). The value dictates the midpoint of the interval, and is N+1 bits long. The extra value bit allows the prediction interval to span power-of-two boundaries, and guarantees that a good prediction can be validated in 2 cycles. FIG. 10A shows a graph plot for example allowed mismatch before error versus prediction 1000, according to some embodiments. As shown, without half stepping, some critical values have zero prediction margin—with only a single count of margin they have a 50/50 chance of producing an error. With the half steps, the minimum tolerance is a quarter of an lower significant bits (LSB) (1/(2**N+2)). This is not zero margin.

For example, the prediction “0.51, 2 bits” gives the subinterval (0.5, 0.625). It is much closer to its lower bound than it's upper bound (0.01 vs 0.115). The subinterval (0.4375, 0.5625) is the same width, but is much more centered (0.0725, 0.0525). This example allows for 5 times as much prediction to value mismatch.

The techniques described herein can be used for SAR ADCs. Required data rate can be less than half of the maximum rate the SAR can handle. Benefit increases as unpredictable signal bandwidth decreases. Prior knowledge of the signal can be useful. The simplest form of prior knowledge could be that the signal bandwidth can be known to be less than the Nyquist limit by some known factor. These techniques can be broadly useful in any oversampling application.

FIG. 10B is a schematic diagram of example plots of intervals update in a traditional SAR (left, labeled 1002), an improved SAR with good prediction (middle, labeled 1004) and an improved SAR with bad prediction (right, labeled 1006). The Y axis is the range of the ADC. Lowest code at the bottom, highest code at the top. The X axis is time. Each conversion cycle is shown as a column, with single column gaps in between. Yellow is the area where the value still could be. Red covers the values that the previous step eliminated. Blue is the predicted interval. The middle bold line indicates the value of the DAC that drives the comparator.

In the traditional example, the SAR assumes that the input is within it's input range, so everything is yellow. The SAR set's it's comparison DAC to midrange and sees that the value is higher than mid range. The SAR continues on its binary search until the SAR finds the value. The example shows a 6 bit SAR taking 6 time steps. The seventh time step just shows that it has found the correct answer.

In the middle example, the SAR is given an enhanced assumption that the value is in the blue region. The SAR is not certain, so the rest of the area is yellow. The first comparison is the middle of the blue range. In this case it is higher, so we know the lower area is red. The next comparison is at the top of the blue range. Since the true value is lower, it is known that the prediction was useful and system can continue as normal. In this example, it took four time steps to find the same answer previously found in six time steps. In the last example, the SAR is given an incorrect assumption. It takes two comparisons to discover that the assumption was incorrect. In some embodiments, the SARs used are 10-14 bits. The example uses 6 bits for illustration.

### Example Model for Adaptive AFE with MA and PLI Detection and Compensation

In some embodiments, the example model can be used to implement adaptive AFE with MA and PLI algorithms described above (e.g., FIG. 7A-8C). The example model can be used to implement simulations. The initial code sets up the input signal (synthetic or loaded from the files in the same sub-folder), enables or disables some artifacts and calls two functions that runs the adaptive AFE, and run a traditional AFE with analog integrators as DC servo loop and no PLI compensation. Finally, a run test code plots various waveforms.

The example model includes one main function implementing the adaptive AFE where analog input signals are passed to it for processing the CFBINA, PGA, ADC, DAC and all the algorithms and state machines. Parameters set the analog and digital simulations parameters, which are stored in another m-file. The simulations can be run using a main simulation file that calls the functions. This test file runs the adaptive AFE PLI function with the preloaded parameters. It feeds the AFE either a synthetic input signal or a signal loaded from the internal EMG band collected data. In some embodiments, the EMG band collected data is generated from signals received at one or more electrodes coupled to the wrist-wearable device held against the skin of a user wearing the wrist-wearable device. The data is collected and put into a dataset that can be used in the requisite algorithms.

The EMG band data files are stored within the same folder extracted from the band data. The beginning of the code defines input signal sand continuous time sample rate a signal definition parameter signal definitions value can be set manually to load the following different types of input signals such as: 0=Synthetic sinusoidal signal; 1=Load MA from EMG database; 2=Synthetic PLI+some synthetic MA; 3=Load PLI from EMG database; 4=Combine loaded MA and PLI from EMG database (synthetically combined). Additional artifacts, such as slow moving baseline, electrode offset, offset jump and EMG gesture, can also be added or removed.

The code can run multiple models, including one of the adaptive AFE and one of a traditional AFE without any algorithms but only using an analog fixed bandwidth DC servo loop. The results of both AFE's including passing of some analog and digital signals and AFE outputs and internal algorithm parameters of the adaptive AFE are then passed back to the simulation script for plotting. The adaptive AFE PLI code runs nested loops going over samples of the passed analog signals into it and then per instances of time when sampling instances of ADC should occur the ADC conversion as well as the MA and PLI algorithm calculations are performed. A separate loop runs in parallel on the oversampling clock of the hybrid DAC's sigma delta modulator. The proceeding graph illustrates visually how these loops and calculations occur across the code. The code has comments and descriptions by as much as possible built into it.

### Example Embodiments

Turning now to some example embodiments of the methods, circuits, devices, and systems described earlier.

(A1) In some embodiments an apparatus (e.g., AFE architecture 100, FIG. 1; the example adaptive AFE system 900, FIG. 9) for processing biopotential signals is disclosed herein. The apparatus includes an analog frontend circuit comprising an instrumentation amplifier (INA) (e.g., instrumentation amplifier 104) and an analog-to-digital circuit (ADC) (e.g., ADC 120) in a forward signal path. The apparatus further includes a digital circuit coupled to the analog frontend circuit and configured to receive input from the ADC. The digital circuit includes an adaptive baseline tracking and compensation circuit (e.g., a subsystem 126) configured to track and compensate motion artifact driven changes, a power line interference (PLI) detection and compensation circuit (e.g., compensation algorithm 124) configured to (i) track a desired number (e.g., up to 7) of PLI harmonics, and (ii) track magnitude, phase, and frequency for the PLI harmonics in real time, a digital-to-analog converter (DAC) circuit (e.g., DAC 114) configured to combine output of the adaptive baseline tracking and compensation circuit and the PLI detection and compensation circuit to output an analog output, and a passive filter (e.g., low-pass filtered output 112) configured to receive the analog output and drive an analog compensation signal at an input of the INA to counteract present artifacts at the input.

(A2) In some embodiments of A1, the instrumentation amplifier is a current-feedback instrumentation amplifier (CFBINA) (e.g., trans-conductors (gm stages) 106-2 and 106-4) with two input stages in the form of trans-conductors.

(A3) In some embodiments of A2, feedback is closed in the current domain past the two input stages, the input and feedback current signals are subtracted and fed to a transimpedance TIA output stage (e.g., transimpedance TIA output stage 108).

(A4) In some embodiments of A2 or A3, an input stage dedicated to feedback is configured to receive a first input from a feedback network (e.g., R2 and R1) and a second input from a low-pass filtered output of the digital-to-analog converter (DAC) circuit.

(A5) In some embodiments of any of A2-A4, the CFBINA's output subsequently drives a programmable gain amplifier (PGA) (e.g., PGA 118) whose output drives the input of the analog-to-digital converter (ADC) that digitizes conditioned analog signal.

(A6) In some embodiments of any of A1-A5, the ADC output, after adaptation and compensation for artifacts, contains a digitized EMG signal (e.g., the digitized EMG signal 122).

(A7) In some embodiments of any of A1-A6, the analog frontend circuit further comprises a programmable gain amplifier (PGA) configured to have a gain of approximately 10 (V/V) and a bandwidth of 1 kHz.

(A8) In some embodiments of any of A1-A7, the PGA's gain is varied using gain adaptation after fast motion artifact recovery.

(A9) In some embodiments of any of A1-A8, the ADC is a 12-bit ADC architecture with a full scale of a 1.8 V supply and the sample rate of the ADC is 20 KHz.

(A10) In some embodiments of any of A1-A9, the DAC is configured to have a full-scale dynamic range to cover electrode offset (e.g., +/−300 mV), slow baseline movements due to motion artifact (e.g., <5 mV), fast transients of motion artifact (e.g., <5 mV), and the PLI (e.g., <5 mV).

(A11) In some embodiments of any of A1-A10, the DAC is configured to have a total dynamic range of 1 V.

(A12) In some embodiments of any of A1-A11, the DAC is a 20-bit DAC.

(A13) In some embodiments of any of A1-A12, the DAC is a hybrid DAC comprising a Nyquist rate DAC and a delta sigma modulated DAC.

(A14) In some embodiments of A13, the Nyquist rate DAC comprises an One-Time Programmable DAC configured to operate at an oversampled sample rate of the delta sigma modulated DAC.

(A15) In some embodiments of A14, the hybrid DAC (an example of which is shown and described above in reference to FIG. 4A) is a moderate resolution DAC (e.g., the DAC 402; a DAC in the 8-bit to 12-bit range) where (i) a predetermined number of its most significant bits (MSBs) (e.g., 9 to 10 bits) cover larger and less sensitive portions of the DAC's dynamic range for large electrode offset compensation, and (ii) the rest of its lower significant bits (LSBs) (e.g., 2 to 3 bits) is driven by means of a delta-sigma modulator (e.g., the delta sigma modulator 404) to implement noise shaping and oversampling to effectively increase their resolution.

(A16) In some embodiments of A13, output currents of the Nyquist rate DAC (e.g., 402) and the delta sigma modulated DAC (e.g., 404) are summed up (e.g., 414) in current domain and filtered through an output stage transconductance (e.g., a combination of current-to-voltage and filter in one stage). As shown in FIG. 4A.

(A17) In some embodiments of A16, the Nyquist rate DAC comprises a One-Time Programmable DAC with the DAC output in the voltage domain, and a passive low-pass filter (LPF) is used for reconstruction filtering.

(A18) In some embodiments of A17, the DAC includes a code segmentation circuit configured to receive output (e.g., the floating point output 418, FIG. 4B) of a motion artifact tracking and PLI tracking circuit and split (e.g., roll-over correction mechanism 422) the output into a combination of M bits of coarse code and N bits of fine code. The fine code is extracted by calculating the modulus of division of the output by 2\hat{ }N, and the coarse code is computed as a difference between a floating point value corresponding to the output and the fine code. The apparatus further includes a hybrid-DAC circuit including a moderate DAC (e.g., the DAC 402) configured to convert the M bits of coarse code and a delta-sigma modulator (e.g., delta-sigma modulator 404) configured to implement noise sampling and oversampling on the N bits of fine code. The delta-sigma modular including a detection algorithm configured to avoid roll-over issues related to delta sigma modulator crossing coarse code between two adjacent segments.

(A19) In some embodiments of any of A1-A18, the passive filter is a low pass filter (e.g., the LPF 412) configured to smooth out fast switching noise of the DAC's delta sigma modulation. The noise is caused by high pass modulated quantization noise of the DAC.

(A20) In some embodiments of any of A1-A18, the adaptive baseline tracking and compensation circuit (e.g., 600, FIG. 6A) includes a bank of multi rate filters (e.g., multi rate filters 616) configured to receive input from the ADC (e.g., ADC 608) to examine signal content in different bands, a rate of change detection circuit (e.g., rate of change extraction block 618) coupled to the bank of multi rate filters, the rate of change determination circuit configured to receive output of the bank of multi rate filters and determine a rate of change for the output, and a state machine and timer circuit (e.g., the block composed of state machine and timers 624) coupled to the rate of change detection circuit and configured to, in accordance with a determination that the rate of change exceeds predetermined adaptive hysteresis thresholds, adjust a bandwidth control knob of a baseline tracking integrator (e.g., the integrator 614).

(A21) In some embodiments of A20, the baseline tracking integrator locks to DC electrode offset from the ADC and during stable situations and passes (e.g., <20 Hz) baseline movements together with the DC to the DAC (e.g., the compensation path DAC 612).

(A22) In some embodiments of A21, the adaptive hysteresis thresholds and the state machine and timer circuit define runaway fast liftoff events, wherein after the runaway fast liftoff events occur, the baseline tracking integrator's bandwidth is opened up to allow the fast transition to also pass the DAC, wherein after the runaway situation passes, as governed the baseline tracking integrator's bandwidth is gradually reduced back to the stable level.

(A23) In some embodiments of A20, one or more parameters of the adaptive baseline tracking and compensation circuit are programmable, wherein the one or more parameters is selected from the group consisting of: filter rates, thresholds, adaptiveness of thresholds, timers, and gains.

(A24) In some embodiments of any of A1-A23, the adaptive baseline tracking and compensation circuit comprises an adaptive baseline tracking circuit and an adaptive bandwidth baseline compensation circuit, wherein the adaptive baseline tracking circuit is configured to determine if a predetermined large and fast event has occurred and accordingly adjust bandwidth of the adaptive compensation circuit, the adaptive bandwidth baseline compensation circuit is configured to track DC electrode offset from the ADC and slow moving baseline.

(A25) In some embodiments of A24, the adaptive baseline tracking circuit is configured to receive the input from the ADC and pass it through three parallel multi-rate filters, the bandwidth of the filters are programmable, and each filter is a second order Butterworth transfer function with bandwidths of high, low, and ultra-low (e.g., 2 kHz, 50 Hz and 20 Hz), respectively.

(A26) In some embodiments of any of A1-A25, the rate of change detection circuit is configured to subtract output from a high filter and a low filter of the bank of multi rate filters, calculate an absolute value of that difference and gain up by a digitally programmable gain to generate the rate of change signal.

(A27) In some embodiments of any of A1-A26, the state machine and timer circuit comprises a hysteresis comparator (e.g., the hysteresis comparator 626, FIG. 6B) configured to use an adaptive thresholding function for fast change detection based on the rate of change signal, the hysteresis comparator is configured to use two thresholds including an upper crossing threshold and a lower crossing threshold.

(A28) In some embodiments of A27, the two thresholds are calculated based on digitally programmable threshold and hysteresis values and output of a running average filter (e.g., the adaptive hysteresis thresholds 620 and fast change detection 622, FIG. 6B) that filters the rate of change signal, thereby allowing the thresholds to slowly track the average of noise and reduce the effect of false triggering.

(A29) In some embodiments of A28, the length of the running average filter is programmable by a separate parameter.

(A30) In some embodiments of any of A27-A29, the hysteresis comparator output is a fast tracking flag signal that goes up when the upper crossing threshold is crossed upwards and goes back down when the lower crossing threshold is crossed downwards, the flag signal triggers a state machine with embedded timers that are programmable, the state machine is configured to, in accordance with a determination that the rate of change goes higher than the upper crossing threshold, reset a fast track monitoring timer and set a fast track flag high and in accordance with a determination that the rate of change drops below the lower threshold, remove the fast track flag high only if fast tracker time kept by the fast track monitoring timer has elapsed and in accordance with a determination that the fast tracker time has not elapsed, continue to update a counter, and keep the fast track flag high.

(A31) In some embodiments of A30, the adaptive baseline tracking circuit is configured to stay in a fast tracking mode by the amount of time defined by the fast track monitoring time, and after the fast track monitoring timer elapses, transition to gradually settle to an ultra-low bandwidth baseline tracking by transitioning through a mid-level slow tracking bandwidth, the amount of time spent in this mode is governed by a separate programmable timer, after that timer elapses the adaptive baseline tracking circuit goes to a slowest tracking bandwidth, in accordance with a determination that a fast moving event occurs during any of these phases, the fast track flag is immediately raised and the state machine rests back to the fast tracking mode.

(A32) In some embodiments of A31, the adaptive baseline tracking circuit is configured to output a controlled selection signal that is input to a multiplexer (e.g., 630) that is configured to feed the adaptive bandwidth baseline compensation circuit with adaptive bandwidths corresponding to the slowest tracking bandwidth, the mid-level slow tracking bandwidth, and the fast tracking mode.

(A33) In some embodiments of any of A1-A32, the adaptive baseline tracking and compensation circuit further comprises a code stitching circuit (e.g., the block 656) configured to remove glitches by delaying output of the ADC by a predetermined number of samples before passed out.

(A34) In some embodiments of any of A1-A33, the PLI detection and compensation circuit (e.g., PLI algorithm block 124, FIG. 1, FIG. 7) is configured to receive, from the ADC, a signal contaminated by PLI, apply a narrowband band-pass filter (e.g., the IIR BPF 716) and use a lattice notch filter (e.g., lattice notch filter 718) to estimate the signal's frequency, lock frequency, phase, and amplitude of a bank of digital oscillators (e.g., M parallel running oscillators 720), each oscillator corresponds to a harmonic based on the estimated frequency, combine output (e.g., summation 722) of the digital oscillators and compute an error based on the output and the signal, and feedback the error to a recursive least squares (RLS) circuit (e.g., the RLS algorithm 724) for amplitude and phase adaptation, the RLS circuit is configured to drive the DAC with a correct PLI signal such that the PLI at the ADC output converges to zero.

(A35) In some embodiments of A34, the narrowband band-pass filter is a bandpass function, a first order filter, with bandwidth frequency set at a first frequency (e.g., the first frequency range can include 40 Hz to 400 Hz).

(A36) In some embodiments of any of A34-A35, the narrowband band-pass filter is a bandpass function, a first order filter, with bandwidth frequency set at approximately 100 Hz.

(A37) In some embodiments of any of A34-A36, the PLI detection and compensation circuit comprises an attenuation circuit (e.g., the PLI_att attenuation 714) configured to receive the signal from the ADC, generate an attenuated signal based on a predetermined attenuation factor, and pass the attenuated signal to the narrowband band-pass filter.

(A38) In some embodiments of any of A34-A37, a plurality of modes for parameters control convergence speed of the lattice notch filter (e.g., lattice notch filter 718) and the RLS circuit to thereby switch to a slow convergence mode when a motion artifact algorithm is active and then switch back to a fast convergence mode when there are no motion artifacts.

(A39) In some embodiments of A38, the plurality of modes for the parameters correspond to different levels of convergence speed based on a severity of the motion artifacts identified.

(A40) In some embodiments of any of A1-A39, the ADC is a successive approximation register analog-to-digital converters (SAR ADC) (an example of which is described above in reference to FIGS. 10A and 10B) comprising an adaptive prediction circuit configured to predict a value for the SAR ADC, and in accordance with a determination that the value matches an input, skip one or more early cycles of the SAR ADC, skipping the one or more early cycles allows the SAR to idle in low power mode.

(A41) In some embodiments of A40, the adaptive prediction circuit is further configured to in accordance with a determination that the value does not match the input, using the predicted value for skipping the one or more cycles for a subsequent conversion.

(A42) In some embodiments of A40-A41, the adaptive prediction circuit is configured to use a prediction interval defined by a depth (N) and a value (C), the value C defines the midpoint of the interval and is N+1 bits long.

(B1) In some embodiments, an apparatus for processing biopotential signals, the apparatus includes a plurality of analog correlators (e.g., correlators 802-2, . . . , 802-18, FIG. 8A), each analog correlator configured to receive time-series analog signals from an electrode (e.g., electrodes 804-2, 804-4 and 804-6) of a biopotential acquisition device, and correlate the time-series analog signals with a respective filter impulse response template to identify a respective degree of correlation, and a plurality of comparators (e.g., comparators 806-2, . . . , 806-18), each comparator coupled to a respective analog correlator and configured to detect peaks in the respective degree of correlation.

(B2) In some embodiments of B1, the respective filter impulse response template corresponds to an expected waveform of gesture obtained through trained or training model and stored in a local memory coupled to the plurality of analog correlators, wherein the plurality of analog correlators is further configured to retrieve the expected waveform from the local memory.

(B3) In some embodiments of B1 or B2, the plurality of analog correlators comprises analog 1-D correlators configured to operate in charge, voltage or current domain.

(B4) In some embodiments of any of B1-B3, each analog correlator is configured to correlate the time-series analog signals by applying a respective quantized weight to the time-series analog signals at a predetermined sample rate, wherein the respective quantized weight comprises a coarsely quantized weight that is quantized in amplitude and/or time.

(B5) In some embodiments of any of B1-B4, each comparator is a single-bit comparator configured to compare the respective degree of correlation with a respective threshold and output a respective digital value.

(B6) In some embodiments of any of B1-B5, the plurality of analog correlators is configured to include a predetermined number of correlator weights per channel per gesture, for a predetermined set of target gestures.

(B7) In some embodiments of any of B1-B6, the respective quantized weight has a size of 2 bits.

(B8) In some embodiments of any of B1-B7, the respective filter impulse response template corresponds to a predetermined number of samples.

(B9) In some embodiments of any of B1-B9, a first set of analog correlators of the plurality of analog correlators is coupled to a first electrode and a second set of analog correlators of the plurality of analog correlators is coupled to a second electrode, a first set of comparators of the plurality of comparators coupled to the first set of analog correlators is configured to output a first binary output (e.g., correlator 808-2) corresponding to a first gesture, a second set of comparators of the plurality of comparators coupled to the second set of analog correlators is configured to output a second binary output (e.g., correlator 808-4) corresponding to a second gesture.

(B10) In some embodiments of B9, the first binary output and the second binary output are combined to generate an interrupt signal (e.g., interrupt 810), the apparatus further comprising a fusion model (e.g., fusion model 812), wherein the fusion model is configured to be woken by the interrupt signal.

(B11) In some embodiments of B10, the fusion model is a neural network, a support vector machine, a decision tree, a Bayesian inference engine, or an ensemble of such classification techniques, which is configured to detect one or more features in the time-series analog signals.

(B12) In some embodiments of B11, the one or more features correspond to a wake-up, lift-off, or one or more gestures (e.g., gesture classification 814).

(B13) In some embodiments of B12, the fusion model is coupled to a circuit configured to reduce interference noise and mitigate saturation in biopotential signals measured by the biopotential acquisition device.

(C1) In some embodiments an apparatus for processing biopotential signals includes a motion artifact detection circuit configured to detect a presence of motion artifact in a biopotential signal and a motion artifact removal circuit configured to adaptively adjust a baseline tracking integrator's bandwidth in a feedback loop to remove the motion artifact in the biopotential signal.

(C2) In some embodiments of C1, the motion artifact detection circuit is configured to use voltage rate of change in time domain to detect the motion artifact.

(C3) In some embodiments of C1 or C2, the motion artifact detection circuit comprises a digital filter, adder and comparator.

(C4) In some embodiments of any of C1-C3, the motion artifact removal circuit comprises a programmable digital filter, state machine and counters for the baseline tracking integrator.

(C5) In some embodiments of any of C1-C4, the motion artifact detection circuit is configured to have three levels of detection which correspond to different corner frequencies of the baseline tracking integrator, the different frequencies correspond to a different level of the motion artifact and extreme lift-off events.

(C6) In some embodiments of C5, the motion artifact spectrum is under 30 Hz and goes up to a predetermined high frequency corresponding to an extreme lift-off event.

(C7) In some embodiments of any of C1-C6, the motion artifact is caused by an electrode slipping on skin or lifting off to produce undesirable artifacts including spikes in the biopotential signal, which take longer than a predetermined amount of time to settle back into a function post motion artifact event.

In another aspect, some embodiments include a computing system (e.g., a wearable device (such as a wrist-wearable device or arm-worn device), intermediary device (which can be configured to perform processor-intensive operations for a system that includes a wrist-wearable device and/or a head-worn wearable device), or combination thereof) including any of the circuits, apparatuses, or biopotential-acquisition systems described herein (e.g., A1-A42, B1-B13, and C1-C7 above).

Any data collection performed by the devices described herein and/or any devices configured to perform or cause the performance of the different embodiments described above in reference to any of the Figures, hereinafter the “devices,” is done with user consent and in a manner that is consistent with all applicable privacy laws. Users are given options to allow the devices to collect data, as well as the option to limit or deny collection of data by the devices. A user is able to opt-in or opt-out of any data collection at any time. Further, users are given the option to request the removal of any collected data.

The devices described above are further detailed below, including wrist-wearable devices, headset devices, systems, and haptic feedback devices. Specific operations described above may occur as a result of specific hardware, such hardware is described in further detail below. The devices described below are not limiting and features on these devices can be removed or additional features can be added to these devices.

### Example Extended-Reality Systems

FIGS. 11A, 11B, 11C-1, and 11C-2, illustrate example XR systems that include AR and MR systems, in accordance with some embodiments. FIG. 11A shows a first XR system 1100a and first example user interactions using a wrist-wearable device 1126, a head-wearable device (e.g., AR device 1128), and/or a HIPD 1142. FIG. 11B shows a second XR system 1100b and second example user interactions using a wrist-wearable device 1126, AR device 1128, and/or an HIPD 1142. FIGS. 11C-1 and 11C-2 show a third MR system 1100c and third example user interactions using a wrist-wearable device 1126, a head-wearable device (e.g., an MR device such as a VR device), and/or an HIPD 1142. As the skilled artisan will appreciate upon reading the descriptions provided herein, the above-example AR and MR systems (described in detail below) can perform various functions and/or operations.

The wrist-wearable device 1126, the head-wearable devices, and/or the HIPD 1142 can communicatively couple via a network 1125 (e.g., cellular, near field, Wi-Fi, personal area network, wireless LAN). Additionally, the wrist-wearable device 1126, the head-wearable device, and/or the HIPD 1142 can also communicatively couple with one or more servers 1130, computers 1140 (e.g., laptops, computers), mobile devices 1150 (e.g., smartphones, tablets), and/or other electronic devices via the network 1125 (e.g., cellular, near field, Wi-Fi, personal area network, wireless LAN). Similarly, a smart textile-based garment, when used, can also communicatively couple with the wrist-wearable device 1126, the head-wearable device(s), the HIPD 1142, the one or more servers 1130, the computers 1140, the mobile devices 1150, and/or other electronic devices via the network 1125 to provide inputs.

Turning to FIG. 11A, a user 1102 is shown wearing the wrist-wearable device 1126 and the AR device 1128 and having the HIPD 1142 on their desk. The wrist-wearable device 1126, the AR device 1128, and the HIPD 1142 facilitate user interaction with an AR environment. In particular, as shown by the first AR system 1100a, the wrist-wearable device 1126, the AR device 1128, and/or the HIPD 1142 cause presentation of one or more avatars 1104, digital representations of contacts 1106, and virtual objects 1108. As discussed below, the user 1102 can interact with the one or more avatars 1104, digital representations of the contacts 1106, and virtual objects 1108 via the wrist-wearable device 1126, the AR device 1128, and/or the HIPD 1142. In addition, the user 1102 is also able to directly view physical objects in the environment, such as a physical table 1129, through transparent lens(es) and waveguide(s) of the AR device 1128. Alternatively, an MR device could be used in place of the AR device 1128 and a similar user experience can take place, but the user would not be directly viewing physical objects in the environment, such as table 1129, and would instead be presented with a virtual reconstruction of the table 1129 produced from one or more sensors of the MR device (e.g., an outward facing camera capable of recording the surrounding environment).

The user 1102 can use any of the wrist-wearable device 1126, the AR device 1128 (e.g., through physical inputs at the AR device and/or built-in motion tracking of a user's extremities), a smart-textile garment, externally mounted extremity tracking device, the HIPD 1142 to provide user inputs, etc. For example, the user 1102 can perform one or more hand gestures that are detected by the wrist-wearable device 1126 (e.g., using one or more EMG sensors and/or IMUs built into the wrist-wearable device) and/or AR device 1128 (e.g., using one or more image sensors or cameras) to provide a user input. Alternatively, or additionally, the user 1102 can provide a user input via one or more touch surfaces of the wrist-wearable device 1126, the AR device 1128, and/or the HIPD 1142, and/or voice commands captured by a microphone of the wrist-wearable device 1126, the AR device 1128, and/or the HIPD 1142. The wrist-wearable device 1126, the AR device 1128, and/or the HIPD 1142 include an artificially intelligent digital assistant to help the user in providing a user input (e.g., completing a sequence of operations, suggesting different operations or commands, providing reminders, confirming a command). For example, the digital assistant can be invoked through an input occurring at the AR device 1128 (e.g., via an input at a temple arm of the AR device 1128). In some embodiments, the user 1102 can provide a user input via one or more facial gestures and/or facial expressions. For example, cameras of the wrist-wearable device 1126, the AR device 1128, and/or the HIPD 1142 can track the user 1102's eyes for navigating a user interface.

The wrist-wearable device 1126, the AR device 1128, and/or the HIPD 1142 can operate alone or in conjunction to allow the user 1102 to interact with the AR environment. In some embodiments, the HIPD 1142 is configured to operate as a central hub or control center for the wrist-wearable device 1126, the AR device 1128, and/or another communicatively coupled device. For example, the user 1102 can provide an input to interact with the AR environment at any of the wrist-wearable device 1126, the AR device 1128, and/or the HIPD 1142, and the HIPD 1142 can identify one or more back-end and front-end tasks to cause the performance of the requested interaction and distribute instructions to cause the performance of the one or more back-end and front-end tasks at the wrist-wearable device 1126, the AR device 1128, and/or the HIPD 1142. In some embodiments, a back-end task is a background-processing task that is not perceptible by the user (e.g., rendering content, decompression, compression, application-specific operations), and a front-end task is a user-facing task that is perceptible to the user (e.g., presenting information to the user, providing feedback to the user). The HIPD 1142 can perform the back-end tasks and provide the wrist-wearable device 1126 and/or the AR device 1128 operational data corresponding to the performed back-end tasks such that the wrist-wearable device 1126 and/or the AR device 1128 can perform the front-end tasks. In this way, the HIPD 1142, which has more computational resources and greater thermal headroom than the wrist-wearable device 1126 and/or the AR device 1128, performs computationally intensive tasks and reduces the computer resource utilization and/or power usage of the wrist-wearable device 1126 and/or the AR device 1128.

In the example shown by the first AR system 1100a, the HIPD 1142 identifies one or more back-end tasks and front-end tasks associated with a user request to initiate an AR video call with one or more other users (represented by the avatar 1104 and the digital representation of the contact 1106) and distributes instructions to cause the performance of the one or more back-end tasks and front-end tasks. In particular, the HIPD 1142 performs back-end tasks for processing and/or rendering image data (and other data) associated with the AR video call and provides operational data associated with the performed back-end tasks to the AR device 1128 such that the AR device 1128 performs front-end tasks for presenting the AR video call (e.g., presenting the avatar 1104 and the digital representation of the contact 1106).

In some embodiments, the HIPD 1142 can operate as a focal or anchor point for causing the presentation of information. This allows the user 1102 to be generally aware of where information is presented. For example, as shown in the first AR system 1100a, the avatar 1104 and the digital representation of the contact 1106 are presented above the HIPD 1142. In particular, the HIPD 1142 and the AR device 1128 operate in conjunction to determine a location for presenting the avatar 1104 and the digital representation of the contact 1106. In some embodiments, information can be presented within a predetermined distance from the HIPD 1142 (e.g., within five meters). For example, as shown in the first AR system 1100a, virtual object 1108 is presented on the desk some distance from the HIPD 1142. Similar to the above example, the HIPD 1142 and the AR device 1128 can operate in conjunction to determine a location for presenting the virtual object 1108. Alternatively, in some embodiments, presentation of information is not bound by the HIPD 1142. More specifically, the avatar 1104, the digital representation of the contact 1106, and the virtual object 1108 do not have to be presented within a predetermined distance of the HIPD 1142. While an AR device 1128 is described working with an HIPD, an MR headset can be interacted with in the same way as the AR device 1128.

User inputs provided at the wrist-wearable device 1126, the AR device 1128, and/or the HIPD 1142 are coordinated such that the user can use any device to initiate, continue, and/or complete an operation. For example, the user 1102 can provide a user input to the AR device 1128 to cause the AR device 1128 to present the virtual object 1108 and, while the virtual object 1108 is presented by the AR device 1128, the user 1102 can provide one or more hand gestures via the wrist-wearable device 1126 to interact and/or manipulate the virtual object 1108. While an AR device 1128 is described working with a wrist-wearable device 1126, an MR headset can be interacted with in the same way as the AR device 1128.

Integration of Artificial Intelligence with XR Systems

FIG. 11A illustrates an interaction in which an artificially intelligent virtual assistant can assist in requests made by a user 1102. The AI virtual assistant can be used to complete open-ended requests made through natural language inputs by a user 1102. For example, in FIG. 11A the user 1102 makes an audible request 1144 to summarize the conversation and then share the summarized conversation with others in the meeting. In addition, the AI virtual assistant is configured to use sensors of the XR system (e.g., cameras of an XR headset, microphones, and various other sensors of any of the devices in the system) to provide contextual prompts to the user for initiating tasks.

FIG. 11A also illustrates an example neural network 1152 used in Artificial Intelligence applications. Uses of Artificial Intelligence (AI) are varied and encompass many different aspects of the devices and systems described herein. AI capabilities cover a diverse range of applications and deepen interactions between the user 1102 and user devices (e.g., the AR device 1128, an MR device 1132, the HIPD 1142, the wrist-wearable device 1126). The AI discussed herein can be derived using many different training techniques. While the primary AI model example discussed herein is a neural network, other AI models can be used. Non-limiting examples of AI models include artificial neural networks (ANNs), deep neural networks (DNNs), convolution neural networks (CNNs), recurrent neural networks (RNNs), large language models (LLMs), long short-term memory networks, transformer models, decision trees, random forests, support vector machines, k-nearest neighbors, genetic algorithms, Markov models, Bayesian networks, fuzzy logic systems, and deep reinforcement learnings, etc. The AI models can be implemented at one or more of the user devices, and/or any other devices described herein. For devices and systems herein that employ multiple AI models, different models can be used depending on the task. For example, for a natural-language artificially intelligent virtual assistant, an LLM can be used and for the object detection of a physical environment, a DNN can be used instead.

In another example, an AI virtual assistant can include many different AI models and based on the user's request, multiple AI models may be employed (concurrently, sequentially or a combination thereof). For example, an LLM-based AI model can provide instructions for helping a user follow a recipe and the instructions can be based in part on another AI model that is derived from an ANN, a DNN, an RNN, etc. that is capable of discerning what part of the recipe the user is on (e.g., object and scene detection).

As AI training models evolve, the operations and experiences described herein could potentially be performed with different models other than those listed above, and a person skilled in the art would understand that the list above is non-limiting.

A user 1102 can interact with an AI model through natural language inputs captured by a voice sensor, text inputs, or any other input modality that accepts natural language and/or a corresponding voice sensor module. In another instance, input is provided by tracking the eye gaze of a user 1102 via a gaze tracker module. Additionally, the AI model can also receive inputs beyond those supplied by a user 1102. For example, the AI can generate its response further based on environmental inputs (e.g., temperature data, image data, video data, ambient light data, audio data, GPS location data, inertial measurement (i.e., user motion) data, pattern recognition data, magnetometer data, depth data, pressure data, force data, neuromuscular data, heart rate data, temperature data, sleep data) captured in response to a user request by various types of sensors and/or their corresponding sensor modules. The sensors' data can be retrieved entirely from a single device (e.g., AR device 1128) or from multiple devices that are in communication with each other (e.g., a system that includes at least two of an AR device 1128, an MR device 1132, the HIPD 1142, the wrist-wearable device 1126, etc.). The AI model can also access additional information (e.g., one or more servers 1130, the computers 1140, the mobile devices 1150, and/or other electronic devices) via a network 1125.

A non-limiting list of AI-enhanced functions includes but is not limited to image recognition, speech recognition (e.g., automatic speech recognition), text recognition (e.g., scene text recognition), pattern recognition, natural language processing and understanding, classification, regression, clustering, anomaly detection, sequence generation, content generation, and optimization. In some embodiments, AI-enhanced functions are fully or partially executed on cloud-computing platforms communicatively coupled to the user devices (e.g., the AR device 1128, an MR device 1132, the HIPD 1142, the wrist-wearable device 1126) via the one or more networks. The cloud-computing platforms provide scalable computing resources, distributed computing, managed AI services, interference acceleration, pre-trained models, APIs and/or other resources to support comprehensive computations required by the AI-enhanced function.

Example outputs stemming from the use of an AI model can include natural language responses, mathematical calculations, charts displaying information, audio, images, videos, texts, summaries of meetings, predictive operations based on environmental factors, classifications, pattern recognitions, recommendations, assessments, or other operations. In some embodiments, the generated outputs are stored on local memories of the user devices (e.g., the AR device 1128, an MR device 1132, the HIPD 1142, the wrist-wearable device 1126), storage options of the external devices (servers, computers, mobile devices, etc.), and/or storage options of the cloud-computing platforms.

The AI-based outputs can be presented across different modalities (e.g., audio-based, visual-based, haptic-based, and any combination thereof) and across different devices of the XR system described herein. Some visual-based outputs can include the displaying of information on XR augments of an XR headset, user interfaces displayed at a wrist-wearable device, laptop device, mobile device, etc. On devices with or without displays (e.g., HIPD 1142), haptic feedback can provide information to the user 1102. An AI model can also use the inputs described above to determine the appropriate modality and device(s) to present content to the user (e.g., a user walking on a busy road can be presented with an audio output instead of a visual output to avoid distracting the user 1102).

### Example Augmented Reality Interaction

FIG. 11B shows the user 1102 wearing the wrist-wearable device 1126 and the AR device 1128 and holding the HIPD 1142. In the second AR system 1100b, the wrist-wearable device 1126, the AR device 1128, and/or the HIPD 1142 are used to receive and/or provide one or more messages to a contact of the user 1102. In particular, the wrist-wearable device 1126, the AR device 1128, and/or the HIPD 1142 detect and coordinate one or more user inputs to initiate a messaging application and prepare a response to a received message via the messaging application.

In some embodiments, the user 1102 initiates, via a user input, an application on the wrist-wearable device 1126, the AR device 1128, and/or the HIPD 1142 that causes the application to initiate on at least one device. For example, in the second AR system 1100b the user 1102 performs a hand gesture associated with a command for initiating a messaging application (represented by messaging user interface 1112); the wrist-wearable device 1126 detects the hand gesture; and, based on a determination that the user 1102 is wearing the AR device 1128, causes the AR device 1128 to present a messaging user interface 1112 of the messaging application. The AR device 1128 can present the messaging user interface 1112 to the user 1102 via its display (e.g., as shown by user 1102's field of view 1110). In some embodiments, the application is initiated and can be run on the device (e.g., the wrist-wearable device 1126, the AR device 1128, and/or the HIPD 1142) that detects the user input to initiate the application, and the device provides another device operational data to cause the presentation of the messaging application. For example, the wrist-wearable device 1126 can detect the user input to initiate a messaging application, initiate and run the messaging application, and provide operational data to the AR device 1128 and/or the HIPD 1142 to cause presentation of the messaging application. Alternatively, the application can be initiated and run at a device other than the device that detected the user input. For example, the wrist-wearable device 1126 can detect the hand gesture associated with initiating the messaging application and cause the HIPD 1142 to run the messaging application and coordinate the presentation of the messaging application.

Further, the user 1102 can provide a user input provided at the wrist-wearable device 1126, the AR device 1128, and/or the HIPD 1142 to continue and/or complete an operation initiated at another device. For example, after initiating the messaging application via the wrist-wearable device 1126 and while the AR device 1128 presents the messaging user interface 1112, the user 1102 can provide an input at the HIPD 1142 to prepare a response (e.g., shown by the swipe gesture performed on the HIPD 1142). The user 1102's gestures performed on the HIPD 1142 can be provided and/or displayed on another device. For example, the user 1102's swipe gestures performed on the HIPD 1142 are displayed on a virtual keyboard of the messaging user interface 1112 displayed by the AR device 1128.

In some embodiments, the wrist-wearable device 1126, the AR device 1128, the HIPD 1142, and/or other communicatively coupled devices can present one or more notifications to the user 1102. The notification can be an indication of a new message, an incoming call, an application update, a status update, etc. The user 1102 can select the notification via the wrist-wearable device 1126, the AR device 1128, or the HIPD 1142 and cause presentation of an application or operation associated with the notification on at least one device. For example, the user 1102 can receive a notification that a message was received at the wrist-wearable device 1126, the AR device 1128, the HIPD 1142, and/or other communicatively coupled device and provide a user input at the wrist-wearable device 1126, the AR device 1128, and/or the HIPD 1142 to review the notification, and the device detecting the user input can cause an application associated with the notification to be initiated and/or presented at the wrist-wearable device 1126, the AR device 1128, and/or the HIPD 1142.

While the above example describes coordinated inputs used to interact with a messaging application, the skilled artisan will appreciate upon reading the descriptions that user inputs can be coordinated to interact with any number of applications including, but not limited to, gaming applications, social media applications, camera applications, web-based applications, financial applications, etc. For example, the AR device 1128 can present to the user 1102 game application data and the HIPD 1142 can use a controller to provide inputs to the game. Similarly, the user 1102 can use the wrist-wearable device 1126 to initiate a camera of the AR device 1128, and the user can use the wrist-wearable device 1126, the AR device 1128, and/or the HIPD 1142 to manipulate the image capture (e.g., zoom in or out, apply filters) and capture image data.

While an AR device 1128 is shown being capable of certain functions, it is understood that an AR device can be an AR device with varying functionalities based on costs and market demands. For example, an AR device may include a single output modality such as an audio output modality. In another example, the AR device may include a low-fidelity display as one of the output modalities, where simple information (e.g., text and/or low-fidelity images/video) is capable of being presented to the user. In yet another example, the AR device can be configured with face-facing light emitting diodes (LEDs) configured to provide a user with information, e.g., an LED around the right-side lens can illuminate to notify the wearer to turn right while directions are being provided or an LED on the left-side can illuminate to notify the wearer to turn left while directions are being provided. In another embodiment, the AR device can include an outward-facing projector such that information (e.g., text information, media) may be displayed on the palm of a user's hand or other suitable surface (e.g., a table, whiteboard). In yet another embodiment, information may also be provided by locally dimming portions of a lens to emphasize portions of the environment in which the user's attention should be directed. Some AR devices can present AR augments either monocularly or binocularly (e.g., an AR augment can be presented at only a single display associated with a single lens as opposed presenting an AR augmented at both lenses to produce a binocular image). In some instances an AR device capable of presenting AR augments binocularly can optionally display AR augments monocularly as well (e.g., for power-saving purposes or other presentation considerations). These examples are non-exhaustive and features of one AR device described above can be combined with features of another AR device described above. While features and experiences of an AR device have been described generally in the preceding sections, it is understood that the described functionalities and experiences can be applied in a similar manner to an MR headset, which is described below in the proceeding sections.

### Example Mixed Reality Interaction

Turning to FIGS. 11C-1 and 11C-2, the user 1102 is shown wearing the wrist-wearable device 1126 and an MR device 1132 (e.g., a device capable of providing either an entirely VR experience or an MR experience that displays object(s) from a physical environment at a display of the device) and holding the HIPD 1142. In the third AR system 1100c, the wrist-wearable device 1126, the MR device 1132, and/or the HIPD 1142 are used to interact within an MR environment, such as a VR game or other MR/VR application. While the MR device 1132 presents a representation of a VR game (e.g., first MR game environment 1120) to the user 1102, the wrist-wearable device 1126, the MR device 1132, and/or the HIPD 1142 detect and coordinate one or more user inputs to allow the user 1102 to interact with the VR game.

In some embodiments, the user 1102 can provide a user input via the wrist-wearable device 1126, the MR device 1132, and/or the HIPD 1142 that causes an action in a corresponding MR environment. For example, the user 1102 in the third MR system 1100c (shown in FIG. 11C-1) raises the HIPD 1142 to prepare for a swing in the first MR game environment 1120. The MR device 1132, responsive to the user 1102 raising the HIPD 1142, causes the MR representation of the user 1122 to perform a similar action (e.g., raise a virtual object, such as a virtual sword 1124). In some embodiments, each device uses respective sensor data and/or image data to detect the user input and provide an accurate representation of the user 1102's motion. For example, image sensors (e.g., SLAM cameras or other cameras) of the HIPD 1142 can be used to detect a position of the HIPD 1142 relative to the user 1102's body such that the virtual object can be positioned appropriately within the first MR game environment 1120; sensor data from the wrist-wearable device 1126 can be used to detect a velocity at which the user 1102 raises the HIPD 1142 such that the MR representation of the user 1122 and the virtual sword 1124 are synchronized with the user 1102's movements; and image sensors of the MR device 1132 can be used to represent the user 1102's body, boundary conditions, or real-world objects within the first MR game environment 1120.

In FIG. 11C-2, the user 1102 performs a downward swing while holding the HIPD 1142. The user 1102's downward swing is detected by the wrist-wearable device 1126, the MR device 1132, and/or the HIPD 1142 and a corresponding action is performed in the first MR game environment 1120. In some embodiments, the data captured by each device is used to improve the user's experience within the MR environment. For example, sensor data of the wrist-wearable device 1126 can be used to determine a speed and/or force at which the downward swing is performed and image sensors of the HIPD 1142 and/or the MR device 1132 can be used to determine a location of the swing and how it should be represented in the first MR game environment 1120, which, in turn, can be used as inputs for the MR environment (e.g., game mechanics, which can use detected speed, force, locations, and/or aspects of the user 1102's actions to classify a user's inputs (e.g., user performs a light strike, hard strike, critical strike, glancing strike, miss) or calculate an output (e.g., amount of damage)).

FIG. 11C-2 further illustrates that a portion of the physical environment is reconstructed and displayed at a display of the MR device 1132 while the MR game environment 1120 is being displayed. In this instance, a reconstruction of the physical environment 1146 is displayed in place of a portion of the MR game environment 1120 when object(s) in the physical environment are potentially in the path of the user (e.g., a collision with the user and an object in the physical environment are likely). Thus, this example MR game environment 1120 includes (i) an immersive VR portion 1148 (e.g., an environment that does not have a corollary counterpart in a nearby physical environment) and (ii) a reconstruction of the physical environment 1146 (e.g., table 1129 and cup). While the example shown here is an MR environment that shows a reconstruction of the physical environment to avoid collisions, other uses of reconstructions of the physical environment can be used, such as defining features of the virtual environment based on the surrounding physical environment (e.g., a virtual column can be placed based on an object in the surrounding physical environment (e.g., a trec)).

While the wrist-wearable device 1126, the MR device 1132, and/or the HIPD 1142 are described as detecting user inputs, in some embodiments, user inputs are detected at a single device (with the single device being responsible for distributing signals to the other devices for performing the user input). For example, the HIPD 1142 can operate an application for generating the first MR game environment 1120 and provide the MR device 1132 with corresponding data for causing the presentation of the first MR game environment 1120, as well as detect the user 1102's movements (while holding the HIPD 1142) to cause the performance of corresponding actions within the first MR game environment 1120. Additionally or alternatively, in some embodiments, operational data (e.g., sensor data, image data, application data, device data, and/or other data) of one or more devices is provided to a single device (e.g., the HIPD 1142) to process the operational data and cause respective devices to perform an action associated with processed operational data.

In some embodiments, the user 1102 can wear a wrist-wearable device 1126, wear an MR device 1132, wear smart textile-based garments 1138 (e.g., wearable haptic gloves), and/or hold an HIPD 1142 device. In this embodiment, the wrist-wearable device 1126, the MR device 1132, and/or the smart textile-based garments 1138 are used to interact within an MR environment (e.g., any AR or MR system described above in reference to FIGS. 11A-11B). While the MR device 1132 presents a representation of an MR game (e.g., second MR game environment 1120) to the user 1102, the wrist-wearable device 1126, the MR device 1132, and/or the smart textile-based garments 1138 detect and coordinate one or more user inputs to allow the user 1102 to interact with the MR environment.

In some embodiments, the user 1102 can provide a user input via the wrist-wearable device 1126, an HIPD 1142, the MR device 1132, and/or the smart textile-based garments 1138 that causes an action in a corresponding MR environment. In some embodiments, each device uses respective sensor data and/or image data to detect the user input and provide an accurate representation of the user 1102's motion. While four different input devices are shown (e.g., a wrist-wearable device 1126, an MR device 1132, an HIPD 1142, and a smart textile-based garment 1138) each one of these input devices entirely on its own can provide inputs for fully interacting with the MR environment. For example, the wrist-wearable device can provide sufficient inputs on its own for interacting with the MR environment. In some embodiments, if multiple input devices are used (e.g., a wrist-wearable device and the smart textile-based garment 1138) sensor fusion can be utilized to ensure inputs are correct. While multiple input devices arc described, it is understood that other input devices can be used in conjunction or on their own instead, such as but not limited to external motion-tracking cameras, other wearable devices fitted to different parts of a user, apparatuses that allow for a user to experience walking in an MR environment while remaining substantially stationary in the physical environment, etc.

As described above, the data captured by each device is used to improve the user's experience within the MR environment. Although not shown, the smart textile-based garments 1138 can be used in conjunction with an MR device and/or an HIPD 1142.

While some experiences are described as occurring on an AR device and other experiences are described as occurring on an MR device, one skilled in the art would appreciate that experiences can be ported over from an MR device to an AR device, and vice versa.

Some definitions of devices and components that can be included in some or all of the example devices discussed are defined here for ease of reference. A skilled artisan will appreciate that certain types of the components described may be more suitable for a particular set of devices, and less suitable for a different set of devices. But subsequent reference to the components defined here should be considered to be encompassed by the definitions provided.

In some embodiments example devices and systems, including electronic devices and systems, will be discussed. Such example devices and systems are not intended to be limiting, and one of skill in the art will understand that alternative devices and systems to the example devices and systems described herein may be used to perform the operations and construct the systems and devices that are described herein.

As described herein, an electronic device is a device that uses electrical energy to perform a specific function. It can be any physical object that contains electronic components such as transistors, resistors, capacitors, diodes, and integrated circuits. Examples of electronic devices include smartphones, laptops, digital cameras, televisions, gaming consoles, and music players, as well as the example electronic devices discussed herein. As described herein, an intermediary electronic device is a device that sits between two other electronic devices, and/or a subset of components of one or more electronic devices and facilitates communication, and/or data processing and/or data transfer between the respective electronic devices and/or electronic components.

What is claimed is:

1. An apparatus for processing biopotential signals, the apparatus comprising:
an analog frontend circuit comprising an instrumentation amplifier (INA) and an analog-to-digital circuit (ADC) in a forward signal path;
a digital circuit coupled to the analog frontend circuit and configured to receive input from the ADC, the digital circuit comprising:
an adaptive baseline tracking and compensation circuit configured to track and compensate motion artifact driven changes;
a power line interference (PLI) detection and compensation circuit configured to (i) track a desired number of PLI harmonics, and (ii) track magnitude, phase and frequency for the PLI harmonics in real time;
a digital-to-analog converter (DAC) circuit configured to combine output of the adaptive baseline tracking and compensation circuit and the PLI detection and compensation circuit to output an analog output; and
a passive filter configured to receive the analog output and drive an analog compensation signal at an input of the INA to counteract present artifacts at the input.

2. The apparatus of claim 1, wherein the instrumentation amplifier is a current-feedback instrumentation amplifier (CFBINA) with two input stages, wherein the two input stages are trans-conductors.

3. The apparatus of claim 2, wherein feedback is closed in a current domain past the two input stages, wherein the input and feedback current signals are subtracted and fed to a transimpedance TIA output stage.

4. The apparatus of claim 2, wherein an input stage dedicated to feedback is configured to receive a first input from a feedback network and a second input from a low-pass filtered output of the digital-to-analog converter (DAC) circuit.

5. The apparatus of claim 2, wherein the CFBINA's output subsequently drives a programmable gain amplifier (PGA) whose output drives the input of the analog-to-digital converter (ADC) that digitizes conditioned analog signal.

6. The apparatus of claim 1, wherein the ADC output, after adaptation and compensation for artifacts, contains a digitized EMG signal.

7. The apparatus of claim Error! Reference source not found., wherein the hybrid DAC is a moderate resolution DAC where (i) a predetermined number of its most significant bits (MSBs) cover larger and less sensitive portions of the DAC's dynamic range for large electrode offset compensation, and (ii) the rest of its lower significant bits (LSBs) is driven by means of the delta-sigma modulated DAC to implement noise shaping and oversampling to effectively increase their resolution.

8. The apparatus of claim Error! Reference source not found., wherein the Nyquist rate DAC comprises an One-Time Programmable DAC with the DAC output in the voltage domain, wherein a passive low-pass filter (LPF) is used for reconstruction filtering.

9. The apparatus of claim Error! Reference source not found., wherein the DAC comprises:
a code segmentation circuit configured to:
receive output of a motion artifact tracking and PLI tracking circuit; and
split the output into a combination of M bits of coarse code and N bits of fine code, wherein the fine code is extracted by calculating a modulus of division of the output by 2\hat{ }N, wherein the coarse code is computed as a difference between a floating point value corresponding to the output and the fine code; and
a hybrid-DAC circuit comprising:
a moderate DAC configured to convert the M bits of coarse code; and
a delta-sigma modulator configured to implement noise sampling and oversampling on the N bits of fine code, the delta-sigma modular including a detection algorithm configured to avoid roll-over issues related to delta sigma modulator crossing coarse code between two adjacent segments.

10. The apparatus of claim 1, wherein the passive filter is a low pass filter configured to smooth out fast switching noise of the DAC's delta sigma modulation, wherein the noise is caused by high pass modulated quantization noise of the DAC.

11. The apparatus of claim 1, wherein the adaptive baseline tracking and compensation circuit comprises:
a bank of multi rate filters configured to receive input from the ADC to examine signal content in different bands;
a rate of change detection circuit coupled to the bank of multi rate filters, the rate of change determination circuit configured to receive output of the bank of multi rate filters and determine a rate of change for the output; and
a state machine and timer circuit coupled to the rate of change detection circuit and configured to, in accordance with a determination that the rate of change exceeds predetermined adaptive hysteresis thresholds, adjust a bandwidth control knob of a baseline tracking integrator.

12. The apparatus of claim Error! Reference source not found., wherein the adaptive hysteresis thresholds and the state machine and timer circuit define runaway fast liftoff events, wherein after the runaway fast liftoff events occur, the baseline tracking integrator's bandwidth is opened up to allow the fast transition to also pass the DAC, wherein after the runaway situation passes, as governed the baseline tracking integrator's bandwidth is gradually reduced back to the stable level.

13. The apparatus of claim 1, wherein the adaptive baseline tracking and compensation circuit comprises an adaptive baseline tracking circuit and an adaptive bandwidth baseline compensation circuit, wherein the adaptive baseline tracking circuit is configured to determine if a predetermined large and fast event has occurred and accordingly adjust bandwidth of the adaptive compensation circuit, wherein the adaptive bandwidth baseline compensation circuit is configured to track DC electrode offset from the ADC and slow moving baseline.

14. The apparatus of claim 13, wherein the adaptive baseline tracking and compensation circuit is configured to:
receive an input from the ADC and pass it through three parallel multi-rate filters, wherein bandwidth of the filters are programmable, wherein each filter is a second order Butterworth transfer function with bandwidths of high, low and ultra-low, respectively.

15. The apparatus of claim 11, wherein the rate of change detection circuit is configured to subtract output from a high filter and a low filter of the bank of multi rate filters, calculate an absolute value of that difference and gain up by a digitally programmable gain to generate the rate of change signal.

16. The apparatus of claim 1, wherein the hysteresis comparator output is a fast tracking flag signal that goes up when the upper crossing threshold is crossed upwards and goes back down when the lower crossing threshold is crossed downwards, wherein the flag signal triggers a state machine with embedded timers that are programmable, wherein the state machine is configured to:
in accordance with a determination that the rate of change goes higher than the upper crossing threshold, reset a fast track monitoring timer and set a fast track flag high; and
in accordance with a determination that the rate of change drops below the lower threshold:
remove the fast track flag high only if fast tracker time kept by the fast track monitoring timer has elapsed; and
in accordance with a determination that the fast tracker time has not elapsed, continue to update a counter and keep the fast track flag high.

17. The apparatus of claim 16, wherein the adaptive baseline tracking and compensation circuit is configured to:
stay in a fast tracking mode by a first amount of time defined by the fast track monitoring timer; and
after the fast track monitoring timer elapses, transition to gradually settle to an ultra-low bandwidth baseline tracking by transitioning through a mid-level slow tracking bandwidth,
wherein the amount of time spent in this mode is governed by a separate programmable timer,
wherein after that timer elapses the adaptive baseline tracking and compensation circuit goes to a slowest tracking bandwidth,
wherein, in accordance with a determination that a fast moving event occurs during any of these phases, the fast track flag is immediately raised and the state machine rests back to the fast tracking mode.

18. The apparatus of claim 1, wherein the PLI detection and compensation circuit is configured to:
receive, from the ADC, a signal contaminated by PLI;
apply a narrowband band-pass filter and use a lattice notch filter to estimate the signal's frequency;
lock frequency, phase and amplitude of a bank of digital oscillators, wherein each oscillator corresponds to a harmonic based on the estimated frequency;
combine output of the digital oscillators and compute an error based on the output and the signal; and
feedback the error to a recursive least squares (RLS) circuit for amplitude and phase adaptation, wherein the RLS circuit is configured to drive the DAC with a correct PLI signal such that the PLI at the ADC output converges to zero.

19. An apparatus for processing biopotential signals, the apparatus comprising:
a plurality of analog correlators, each analog correlator configured to:
receive time-series analog signals from an electrode of a biopotential acquisition device; and
correlate the time-series analog signals with a respective filter impulse response template to identify a respective degree of correlation; and
a plurality of comparators, each comparator coupled to a respective analog correlator and configured to detect peaks in the respective degree of correlation.

20. A non-transitory computer-readable storage medium including instructions configured to cause an apparatus to:
obtain a plurality of biopotential signals;
detect a presence of motion artifact in a biopotential signal; and
adaptively adjust a baseline tracking integrator's bandwidth in a feedback loop to remove the motion artifact in the biopotential signal.
